(19) United States
(12) Patent Application Publication
(10) Pub. No.: US 2025/0041445 A1
(43) Pub. Date: Feb. 6, 2025

(54) GENE THERAPY FOR TREATMENT OF MUCOPOLYSACCHARIDOSIS IIIA

(71) Applicants: The Trustees of the University of Pennsylvania, Philadelphia, PA (US); Amicus Therapeutics, Inc., Philadelphia, PA (US)

(72) Inventors: Juliette Hordeaux, Philadelphia, PA (US); James M. Wilson, Philadelphia, PA (US); Leida Rassouli-Taylor, Philadelphia, PA (US); Hung Do, Philadelphia, PA (US); Steven Tuske, Philadelphia, PA (US)

(73) Assignees: The Trustees of the University of Pennsylvania, Philadelphia, PA (US); Amicus Therapeutics, Inc., Philadelphia, PA (US)

(21) Appl. No.: 18/709,415

(22) PCT Filed: Nov. 11, 2022
(86) PCT No.: PCT/US2022/079701

§ 371 Date: May 10, 2024

Related U.S. Application Data
(60) Provisional application No. 63/278,775, filed on Nov. 12, 2021.
(60) Provisional application No. 63/288,293, filed on Dec. 10, 2021.

(51) Int. Cl. A61K 48/00; A61K 38/46; A61P 3/00; C12N 15/86

(52) U.S. Cl. CPC A61K 48/005; A61K 38/46; A61K 48/0075; A61K 48/0083; A61P 3/00; C12N 15/86; C12Y 310/01001; C12N 2750/14143; C12N 2750/14152; C12N 2830/48; C12N 2830/50

(57) ABSTRACT

Provided herein is a recombinant AAV (rAAV) comprising an AAV capsid and a vector genome packaged therein, wherein the vector genome comprises an AAV 5′ inverted terminal repeat (ITR), an engineered nucleic acid sequence encoding a functional hSGSH, optionally comprising stabilizing amino acid changes, a regulatory sequence which direct expression of hSGSH in a target cell, and an AAV 3′ ITR. Also provided is a pharmaceutical composition comprising a rAAV as described herein in a formulation buffer, and a method of treating a human subject diagnosed with MPS IIIA.

### BACKGROUND OF THE INVENTION

Mucopolysaccharidosis type IIIa (MPS IIIa, MPS IIIA, or Sanfilippo syndrome type A), is an autosomal recessive inherited disorder caused by the deficiency of the enzyme N-sulfoglycosamine sulfohydrolase (SGSH), involved in the lysosomal catabolism of the glycosaminoglycans (GAG) heparan sulfate. This deficiency leads to the intracellular accumulation of undegraded heparan sulfate as well as gangliosides GM2 and GM3 in the central nervous system causing neuronal dysfunction and neuroinflammation. The disease manifests first as a cognitive delay around 3 years of age followed by abnormal hyperactive and aggressive behavior. The progression of the disease then leads to a loss of motor and neurological functions during the first decade with death at a median age of 15 years.

Medications are used to relieve symptoms (such as anticonvulsants for seizures) and improve quality of life. Hematopoietic stem cell transplantation does not seem to ameliorate neuropsychological deterioration significantly. Recombinant enzymes for the deficiencies in MPS III are available, but trials in enzyme replacement therapy (ERT) have not been favorable in improving prognosis because the enzymes are not able to enter the central nervous system. See, e.g., Germaine L Defendi. Genetics of Mucopolysaccharidosis Type III. Medscape. Mar. 21, 2014. Changes to the diet do not prevent disease progression, but limiting milk, sugar, and dairy products has helped some people who have excessive mucus.

Various gene therapy approaches have been described as having potential for effective treatment of MPSIIIA. However, current constructs have not achieved the desired level of therapeutic effect and there is not standard treatment or cure for treatment of Sanfilippo syndrome. A continuing need in the art exists for compositions and methods for effective treatment of MPS IIIA.

### SUMMARY OF THE INVENTION

Provided herein is a therapeutic, recombinant, and replication-defective adeno-associated virus (rAAV) comprising an engineered nucleic acid sequence encoding a functional human N-sulfoglycosamine sulfohydrolase (hSGSH) a regulatory sequence which direct expression thereof in a target cell. In one aspect provided herein is an rAAV comprising adeno-associated virus (AAV) capsid and a vector genome, wherein the vector genome comprises an AAV 5′ inverted terminal repeat (ITR), an expression cassette, and an AAV 3′ITR, wherein the expression cassette comprises an engineered nucleic acid sequence encoding a functional human N-sulfoglycosamine sulfohydrolase (hSGSH), wherein the hSGSH coding sequence comprises a signal peptide sequence and a mature hSGSH coding sequence, wherein the mature hSGSH coding has the nucleic acid sequence of sequence of SEQ ID NO: 16 or a nucleic acid sequence which is (a) at least 85% identical thereto which encodes SEQ ID NO: 23; or (b) at least 99% identical thereto which encodes SEQ ID NO: 23 wherein the hSGSH coding sequence is operably linked to regulatory control sequences which direct expression of the hSGSH in a cell. In certain embodiments, the rAAV comprises the mature hSGSH coding sequence of SEQ ID NO: 22 which encodes SEQ ID NO: 23 (hSGSH.A482Y-E488V). In certain embodiments, the signal peptide sequence is a native signal sequence having nucleic acid sequence of SEQ ID NO: 31 or a sequence at least about 95% identical thereto which encodes SEQ ID NO: 32. In certain embodiments, the signal peptide is an exogenous signal peptide, wherein exogenous signal peptide sequence is a BiP signal peptide sequence having the nucleic acid sequence of SEQ ID NO: 29 or a sequence at least about 95% identical thereto which encodes SEQ ID NO: 30. In certain embodiments, the hSHSH has the nucleic acid sequence of SEQ ID NO: 18 or a sequence at least about 95% identical thereto which encodes SEQ ID NO: 19. In certain embodiments, the hSHSH has the nucleic acid sequence of SEQ ID NO: 24 or a sequence at least about 95% identical thereto which encodes SEQ ID NO: 25. In certain embodiments, the hSGSH coding sequence comprising the BiP signal sequence at the amino terminus of mature hSGSH (5′ end of mature hSGSH coding sequence) and vIGF2 peptide at carboxy terminus of mature hSGSH (at 3′ end of mature hSGSH coding sequence) and has the nucleic acid sequence of SEQ ID NO: 20 or a sequence at least about 95% identical thereto which encodes SEQ ID NO: 21. In certain embodiments, the hSGSH coding sequence has the nucleic acid sequence of SEQ ID NO: 26 or a sequence at least about 95% identical thereto which encodes SEQ ID NO: 27. In some embodiments, the regulatory sequences further comprise one or more of a CB7 promoter, Kozak sequence, an intron, an enhancer, a TATA signal and a polyadenylation (polyA) signal sequence, optionally wherein the regulatory sequences further comprise and WPRE element.

In certain embodiments, the vector genome has the nucleic acid sequence (i) CB7.CI.BIP.hSGSHcov1-A482Y_E488V.vIGF2.rBG (SEQ ID NO: 3); (ii) CB7.CI.BIP.hSGSHcov1-A482Y_E488V.vIGF2.WPRE.rBG (SEQ ID NO: 7); (iii) CB7.CI.hSGSHcoV1.rBG (SEQ ID NO: 10); (iv) CB7.CI.hSGSHcoV1-4xmiR183.rBG (SEQ ID NO: 13); (v) CB7.CI.BIP.hSGSHcov1-A482Y_E488V.vIGF2.WPRE.4xmiR182.rBG (SEQ ID NO: 40); (vi) CB7.CI.BIP.hSGSHcov1(A482Y-E488V).vIGF2.WPRE.4xmiR183.rBG (SEQ ID NO: 1); or (vii) CB7.CI.BIP.hSGSHcov1-A482Y_E488V.vIGF2.WPRE.4xmiR182.4xmiR183.rBG (SEQ ID of SEQ ID NO: 14 or a sequence at least 95% identical thereto. In certain embodiments, the capsid is an AAVhu68 capsid, an AAVhu95 capsid, or AAVrh91 capsid.

In a further aspect, provided herein is a composition and pharmaceutical composition comprising a rAAV or a vector as described herein and an aqueous suspension media. In certain embodiments, the rAAV or the composition thereof is for use in the treatment of Mucopolysaccharidosis III A (MPS IIIA or Sanfilippo syndrome type A) and/or improving gait or mobility, reducing tremors, reducing spasms, improving posture, or reducing the progression of vision loss in a subject in need thereof.

In another aspect, a method of treating a subject having MPS IIIA, or ameliorating symptoms of MPS IIIA, or delaying progression of MPS IIIA is provided. The method comprises administrating an effective amount of a rAAV or a vector as described herein to a subject in need thereof. In certain embodiments, a suspension is formulated for intravenous administration, intrathecal administration, intra-cisterna magna administration or intracerebroventricular administration. In certain embodiments, the suspension is formulated for administration at a dose 1×10⁹ GC per gram of brain mass to about 1×10¹³ GC per gram of brain mass.

In certain embodiments, provide herein is a nucleic acid molecule comprising expression cassette selected from SEQ ID NO: 2, 5, 8, 11, and 14. In certain embodiments, the nucleic acid molecule is a plasmid. In certain embodiments, a packaging cell is provided which comprises the expression cassette, vector genome or plasmid.

These and other aspects of the invention are apparent from the following detailed description of the invention.

### BRIEF DESCRIPTION OF THE DRAWINGS

FIG. 1A shows various designed hSGSH constructs, comprising wild-type (WT) mature SGSH coding sequence, and further comprising either endogenous signal sequence or binding immunoglobulin protein (BiP) signal sequence, linker, vIGF2 peptide (i.e., peptide that binds CI-MPR), and/or lysosomal cleavage sequence.

FIG. 1B shows expression levels of SGSH secreted into HEK293 cell media at 3-days post transfection, as analyzed using western blot.

FIG. 1C shows quantified SGSH expression levels (from western blot analysis; FIG. 1B), plotted as normalized SGSH secretion levels.

FIG. 2A shows expression levels of SGSH secreted into HEK293 cell media at 4-days post transfection, as analyzed using western blot.

FIG. 2B shows quantified SGSH expression levels (from western blot analysis; FIG. 2A), plotted as normalized SGSH secretion levels.

FIG. 2C shows schematic representation of further engineered the mature SGSH coding sequence for use in BiP signal sequence- and vIGF2 peptide-comprising constructs.

FIG. 3A shows SGSH enzyme activity from serum samples collected from mice administered with AAVhu68.hSGSH (1×10¹⁰ GC or 1×10¹¹ GC) on Day 7 of the study.

FIG. 3B shows SGSH enzyme activity from serum samples collected from mice administered with AAVhu68.hSGSH (1×10¹¹ GC or 1×10¹¹ GC) on Day 28 of the study.

FIG. 4A shows SGSH enzyme activity from homogenate liver tissue samples collected from mice administered with 1×10¹⁰ GC and 1×10¹¹ GC.

FIG. 4B shows SGSH enzyme activity from homogenate brain tissue samples collected from mice administered with 1×10¹⁰ GC and 1×10¹¹ GC.

FIG. 5A shows expression levels of SGSH as analyzed from liver tissue homogenate samples from mice administered with AAVhu68.hSGSH at doses of 1×10¹⁰ GC and 1×10¹¹ GC.

FIG. 5B shows quantified expression levels of SGSH as analyzed by western blot (shown in FIG. 5A).

FIG. 6A shows SGSH expression and quantification in cortex following administration of AAVhu68.hSGSH with WPRE. FIG. 6B shows SGSH expression and quantification in cerebellum following administration of AAVhu68.hSGSH with WPRE. FIG. 6C shows SGSH expression and quantification in hippocampus following administration of AAVhu68.hSGSH with WPRE. FIG. 6D shows SGSH expression and quantification in brain stem following administration of AAVhu68.hSGSH with WPRE.

FIG. 7A shows results of the SGSH protein concentration, plotted as ng/g, as analyzed from collected liver tissue samples from mice which were administered with AAVhu68.hSGSH at doses of 1×10¹⁰ GC to 1×10¹¹ GC.

FIG. 7B shows correlation between SGSH enzyme activity (as measured using fluorescence assay) and Signal Peptide assay performed on collected liver samples from mice administered with high dose of AAVhu68.hSGSH (FIG. 7A; 1×10¹¹ GC).

FIGS. 8A to 8F shows endpoint analysis of lysosomal compartment reduction as examined via LAMP1 quantification using immunofluorescent and histochemical analysis in MPS IIIA SGSH KO mice administered at a low dose (1×10¹⁰) of AAVhu68.hSGSH. Group 1 (G1)=AAVhu68.CB7.hSGSHcoV1; G2=AAVhu68.CB7.BIP-hSGSHcoV1; G3=AAVhu68.CB7.BIP-hSGSHcoV1-vIGF2; G4=AAVhu68.CB7.BIP-hSGSH (A482Y E488V) coV1; G5=AAVhu68.CB7.BIP-hSGSH (A482Y E488V) coV1-vIGF2; G6=KO mice PBS controls; G7=WT mice PBS controls. FIG. 8A shows mean size of LAMP1-positive cells (μm²) in cerebellum. FIG. 8B shows percent LAMP1-area in cerebellum. FIG. 8C shows mean size of LAMP1-positive cells (μm²) in brain stem. FIG. 8D shows percent LAMP1-area in brain stem. FIG. 8E shows mean size of LAMP1-positive cells (μm²) in cortex. FIG. 8F shows percent LAMP1-area in cortex.

FIGS. 9A to 9F shows endpoint analysis of lysosomal compartment reduction as examined via LAMP1 quantification using immunofluorescent and histochemical analysis in MPS IIIA SGSH KO mice administered at a high dose (1×10¹¹) of AAVhu68.hSGSH. G6 and G7 similar to FIG. 8 (KO and WT PBS controls); G8=AAVhu68.CB7.hSGSHcoV1; G11=AAVhu68.CB7.BIP-hSGSH (A482Y E488V); G12=AAVhu68.CB7.BIP-hSGSH (A482Y E488V) coV1-vIGF2. FIG. 9A shows mean size of LAMP1-positive cells (μm²) in cerebellum. FIG. 9B shows percent LAMP1-area in cerebellum. FIG. 9C shows mean size of LAMP1-positive cells (μm²) in brain stem. FIG. 9D shows percent LAMP1-area in brain stem. FIG. 9E shows mean size of LAMP1-positive cells (μm²) in cortex. FIG. 9F shows percent LAMP1-area in cortex. FIG. 9H shows percent LAMP1-area in cortex for G1, G2 and G3, as defined in FIGS. 8A-8F, at a dose of 2×10¹⁰ GC in 2-3 month old mice, measured at 1 month using the Mann-Whitney test. FIG. 9I shows percent LAMP-1 area in cerebellum for G1, G2 and G3, at a dose of 2×10¹⁰ GC in 2-3 month old mice, measured at 1 month using the Mann-Whitney test. FIG. 9J shows percent LAMP-1 area in hippocampus for G1, G2 and G3, at a dose of 2×10¹⁰ GC in 2-3 month old mice, measured at 1 month using the Mann-Whitney test.

FIGS. 10A to 10C shows SGSH activity and GAG reduction in brain of male and female mice. FIG. 10A shows SGSH activity in brain plotted as activity (nmol/mL/hr). FIG. 10B shows SGSH activity in brain, plotted as log activity. FIG. 10C shows GAG levels in brain plotted as ng GAG(HS) per mg protein.

FIGS. 11A to 11C shows SGSH activity in GAG reduction in spinal cord of male and female mice. FIG. 11A shows SGSH activity in spinal cord plotted as activity (nmol/mL/hr). FIG. 11B shows SGSH activity in spinal cord plotted as log activity. FIG. 11C shows GAG levels in spinal cord plotted as ng GAG(HS) per mg protein.

FIGS. 12A to 12C shows SGSH activity in GAG reduction in liver of male and female mice. FIG. 12A shows SGSH activity in liver plotted as activity (nmol/mL/hr). FIG. 12B shows SGSH activity in liver plotted as log activity. FIG. 12C shows GAG levels in liver plotted as ng GAG(HS) per mg protein.

FIG. 13A shows SGSH activity in serum plotted as activity (nmol/mL/hr) as measure on day 7 of the post ICV injection. FIG. 13B shows SGSH activity in serum plotted as log activity (nmol/mL/hr) on day 7 post ICV injection. FIG. 13C shows SGSH levels activity in plasma plotted as activity (nmol/mL/hr) as measured at one month post ICV injection. FIG. 13D shows SGSH levels activity in plasma plotted as log activity (nmol/mL/hr) as measured at one month post ICV injection.

FIG. 14A shows levels of total GM3 in mouse brain plotted as pmol GM3 per mg protein. FIG. 14B shows levels of total GM3 in mouse brain potted as log scale pmol GM3 per mg protein.

FIG. 15A shows levels of SGSH baseline activity in untreated NHP brain sections plotted as nmol/mg/hr, as measured in medulla, cerebellum, thalamus, frontal cortex. FIG. 15B shows levels of SGSH baseline activity in untreated NHP spinal cord sections plotted as nmol/mg/hr, as measured in spinal cord cervical, thoracic, lumbar, and dorsal root ganglion (DRG) cervical, lumbar and thoracic sections.

FIGS. 15C to 15E shows axonopathy following delivery of the AAVhu68.hSGSH, AAVrh91.hSGSH, or AAVhu68hSGSH with an engineered peptide. FIG. 15C shows dorsal root ganglia (DRG) necrosis average. FIG. 15D shows spinal cord axonopathy average. FIG. 15E shows median nerve axonopathy average.

FIG. 16 shows SGSH transgene expression levels, plotted as AFU (800/700 nm), in neurospheres treated with AAV.hSGSH comprising WT SGSH construct, engineered SGSH construct or engineered SGSH construct further comprising WPRE element in AAV vector genome at various MOI.

FIG. 17A shows SGSH activity in treated mouse brain. FIG. 17B shows GAG (HS) levels in brain. FIG. 17C shows total GM3 in mouse brain. These results show that the addition of WPRE to the engineered candidate rescues expression to levels similar to the WT construct and allows comparable or better storage clearance.

FIG. 18A shows SGSH activity in treated NHP cerebellum brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 18B shows SGSH activity in treated NHP medulla brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 18C shows SGSH activity in treated NHP frontal cortex brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 18D shows SGSH activity in treated NHP thalamus brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE).

FIG. 19A shows SGSH activity in treated NHP spinal cord section (SC cervical), compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 19B shows SGSH activity in treated NHP spinal cord section (SC thoracic), compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 19C shows SGSH activity in treated NHP spinal cord section (SC lumbar), compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE).

FIG. 20A shows SGSH activity in treated NHP plasma. FIG. 20B shows SGSH activity in treated CSF

FIG. 21A shows total anti-hSGSH IgG titer in NHP plasma. FIG. 21B shows total anto-hSGSH titer in CSF.

FIG. 22A shows results of the nerve conduction velocity (NCV), plotted as NP (nerve polarization in Amp) in left median nerve. FIG. 22B shows results of the nerve conduction velocity (NCV), plotted as NP (nerve polarization in Amp) in right median nerve. FIG. 22C shows results of the nerve conduction velocity (NCV), plotted as velocity in left median nerve. FIG. 22D shows results of the nerve conduction velocity (NCV), plotted as velocity in right median nerve.

FIG. 23 shows serum Nfl in treated NHP, which shows that Serum NfL aligns with histopathology axonopathy severity.

### DETAILED DESCRIPTION OF THE INVENTION

Provided herein are composition and methods of compositions useful for the treatment of Mucopolysaccharidosis type IIIa (MPS IIIA) and/or alleviating symptoms of MPS IIIA. These compositions comprise a nucleic acid sequence encoding a functional human N-sulfoglycosamine sulfohydrolase (hSGSH) and a regulatory sequence which direct expression thereof in a target cell, wherein the hSGSH coding sequence comprises a signal peptide sequence and a mature hSGSH coding sequence, wherein the mature hSGSH coding has the nucleic acid sequence of sequence of SEQ ID NO: 16 or a nucleic acid sequence which is (a) at least 85% identical thereto which encodes SEQ ID NO: 23; or (b) at least 99% identical thereto which encodes SEQ ID NO: 23.

In one embodiment, the compositions and methods described herein involve nucleic acid sequences, expression cassettes, vectors, recombinant viruses, other compositions and methods for expression of a functional hSGSH. In another embodiment, the compositions and methods described herein involve nucleic acid sequences, expression cassettes, vectors, recombinant viruses, host cells, other compositions and methods for production of a composition comprising the nucleic acid sequence encoding a functional human SGSH. In yet another embodiment, the compositions and methods described herein involve nucleic acid sequences, expression cassettes, vectors, recombinant viruses, other compositions and methods for delivery of the nucleic acid sequence encoding a functional hSGSH to a subject for the treatment of MPS IIIA. In one embodiment, the compositions and methods described herein are useful for providing a therapeutic level of SGSH into the central nervous system (CNS). Additionally or alternatively, the compositions and methods described herein are useful for providing therapeutic levels of SGSH in the periphery, such as, e.g., blood, liver, kidney, or peripheral nervous system. In certain embodiments, an adeno-associated viral (AAV) vector-based method described herein provides a new treatment option, helping to restore a desired function of SGSH, to alleviate a symptom associated with MPS IIIA, to improve MPS IIIA-related biomarkers, or to facilitate other treatment(s) for MPS IIIA, by providing expression of SGSH protein in a subject in need.

As used herein, the term “a therapeutic level” means an enzyme activity at least about 5%, about 10%, about 20%, about 25%, about 30%, about 35%, about 40%, about 45%, about 50%, about 55%, about 60%, about 65%, about 70%, about 75%, about 80%, about 85%, about 90%, about 95%, about 100%, more than 100%, about 2-fold, about 3-fold, or about 5-fold of a healthy control. Suitable assays for measuring SGSH enzymatic activity are described herein. In some embodiments, such therapeutic levels of SGSH may result in alleviation of the MPS III-A related symptoms; improvement of MPS IIIA-related biomarkers of disease; or facilitation of other treatment(s) for MPS IIIA, e.g., GAG levels in the cerebrospinal fluid (CSF), serum, urine and/or other biological samples; prevention of neurocognitive decline; reversal of certain MPS IIIA-related symptoms and/or prevention of progression of MPS IIIA-related certain symptoms; or any combination thereof.

As used herein, “a healthy control” refers to a subject or a biological sample therefrom, wherein the subject does not have an MPS IIIA disorder. The healthy control can be from one subject. In another embodiment, the healthy control is a pool of multiple subjects.

As used herein, the term “biological sample” refers to any cell, biological fluid or tissue. Suitable samples for use in this invention may include, without limitation, whole blood, leukocytes, fibroblasts, serum, urine, plasma, saliva, bone marrow, cerebrospinal fluid, amniotic fluid, and skin cells. Such samples may further be diluted with saline, buffer or a physiologically acceptable diluent. Alternatively, such samples are concentrated by conventional means.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

As used herein, “disease”, “disorder” and “condition” are Mucopolysaccharidosis type IIIA (MPSIIIA, MPS IIIA, MPS IIIa, also known as Sanfilippo syndrome type A or Sanfilippo type A disease).

As used herein, the term “MPS IIIA-related symptom(s)” or “symptom(s)” refers to symptom(s) found in MPS IIIA patients as well as in MPS IIIA animal models. Such symptoms include but not limited to delayed speech; difficulty with social interactions and communication; sleep disturbances; progressive intellectual disability and the loss of previously acquired skills (developmental regression); seizures and movement disorders; a large head; a slightly enlarged liver (mild hepatomegaly); a soft out-pouching around the belly-button (umbilical hernia) or lower abdomen (inguinal hernia); short stature, joint stiffness, mild dysostosis multiplex, multiple skeletal abnormalities; chronic diarrhea; recurrent upper respiratory infections; recurrent ear infections; hearing impairment; vision problems; Asymmetric septal hypertrophy; Coarse facial features; Coarse hair; Dense calvaria; Dysostosis multiplex; Growth abnormality; Heparan sulfate excretion in urine; GAG accumulation in the cerebrospinal fluid (CSF), serum, urine and/or any other biological samples; abnormal expression and/or enzyme activity of N-acetyl-alpha-D-glucosaminidase (NAGLU) or N-sulfoglycosamine sulfohydrolase (IDUA); accumulation of GM2 and GM3; changed activity in lysosomal enzymes; accumulation of free unesterified cholesterol in the CNS; inflammatory response in the CNS and skeletal tissues; excess hair growth (Hirsutism); Hyperactivity; Ovoid thoracolumbar vertebrae; Splenomegaly; Synophrys; Thickened ribs; hernias; and a wobbly and erratic walk.

“Patient” or “subject” as used herein means a male or female human, dogs, and animal models used for clinical research. In one embodiment, the subject of these methods and compositions is a human diagnosed with MPS IIIA. In certain embodiments, the human subject of these methods and compositions is a prenatal, a newborn, an infant, a toddler, a preschool, a grade-schooler, a teen, a young adult or an adult. In a further embodiment, the subject of these methods and compositions is a pediatric MPS IIIA patient.

Clinical examination and urine tests (excess mucopolysaccharides are excreted in the urine) are the first steps in the diagnosis of an MPS disease. Enzyme assays measuring levels of enzyme activity in the blood, skin cells or a variety of cells are also used to provide definitive diagnosis of MPS IIIA. Various genetic testing detecting a mutation of SGSH associated with MPS IIIA is available. See, e.g., ncbi.nlm.nih.gov/gtr/conditions/-C0086647/; ncbi.nlm.nih.gov/gtr/all/tests/?term=C0086647[DISCUI]. Prenatal diagnosis using amniocentesis and chorionic villus sampling can verify if a fetus is affected with the disorder. Genetic counseling can help parents who have a family history of the mucopolysaccharidoses determine if they are carrying the mutated gene that causes the disorders. See, e.g., A Guide to Understanding MPS III, National MPS Society, 2008, mpssociety.org/learn/diseases/mps-iii/.

### N-Sulfoglycosamine Sulfohydrolase (SGSH)

As used herein, the terms “N-sulfoglycosamine sulfohydrolase” and “SGSH” are used interchangeably with heparan-N-sulfatase, HNS. The invention includes any variant of SGSH protein expressed from the nucleic acid sequences provided herein, or a functional fragment thereof, which restores a desired function, ameliorates a symptom, improves symptoms associated with a MPS IIIA-related biomarker, or facilitate other treatment(s) for MPS IIIA when delivered in a composition or by a method as provided herein. Examples of a suitable biomarker for MPSIII includes that described in WO 2017/136533, which is incorporated herein by reference.

As used herein, the term “functional SGSH” means an enzyme having the amino acid sequence of the full-length wild-type (native) human SGSH (as shown in SEQ ID NO: 36 and UniProtKB accession number: P51688), a variant thereof, a mutant thereof with a conservative amino acid replacement, a fragment thereof, a full-length or a fragment of any combination of the variant and the mutant with a conservative amino acid replacement, which provides at least about 10%, at least about 20%, at least about 30%, at least about 40%, at least about 50%, at least about 60%, at least about 70%, at least about 75%, at least about 80%, at least about 90%, or about the same, or greater than 100% of the biological activity level of normal human SGSH. The full-length wild-type (native) human SGSH (SEQ ID NO: 36) comprises a native signal (or leader) peptide and a mature hSGSH (SEQ ID NO: 17). In some embodiment, a functional SGSH refers to a wild-type protein with sequence of SEQ ID NO: 36. In certain embodiments, a functional hSGSH comprises a native leader sequence. In certain embodiments, a functional hSGSH comprises an exogenous leader sequence. In certain embodiments, a functional hSGSH comprises an exogenous leader sequence which is an exogenous human immunoglobulin heavy chain binding protein BIP leader sequence (BiP or Bip). See also, WO2012/071422 A2 and U.S. Pat. No. 9,279,007, which are incorporated herein by reference. In certain embodiments, the functional hSGSH further comprises a peptide that enables endocytosis, wherein the peptide binds the CI-MPR. In certain embodiments, the peptide that binds to CI-MPR is a vIGF2 peptide. In certain embodiments, a functional hSGSH is a fusion protein comprising an exogenous leader peptide sequence which is BIP leader sequence, a mature hSGSH, and a vIGF2 peptide connected via a linker. In certain embodiments, the functional hSGSH comprises mature hSGSH having at least one or more mutations that enhances stability and/or expression of the hSGSH in cell (i.e., stabilizing amino residue changes). In certain embodiments, a functional hSGSH is a fusion protein comprising an exogenous leader peptide sequence which is BIP leader sequence, a mature hSGSH comprising stabilizing amino acid residue changes A482Y and E488V, and a vIGF2 peptide connected via a linker.

As used herein, the “conservative amino acid replacement” or “conservative amino acid substitutions” refers to a change, replacement or substitution of an amino acid to a different amino acid with similar biochemical properties (e.g., charge, hydrophobicity and size), which is known by practitioners of the art. Also see, e.g., FRENCH et al. What is a conservative substitution?Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

In certain embodiments, a functional SGSH is an SGSH variants which includes A482Y, which consists of the amino acid sequence of SEQ ID NO: 36 with a tyrosine (Tyr, Y) at the 488th amino acid instead of alanine (Ala, A) in the wild-type; and E488V, which consists of the amino acid sequence of SEQ ID NO: 36 with a valine (Val, V) at the 488th amino acid instead of glutamic acid (Glu, E) in the wild-type. Additional examples of SGSH variants may include those predicted by bioinformatic tools available to one of skill in the art. See, e.g., Ugrinov K G et al. A multiparametric computational algorithm for comprehensive assessment of genetic mutations in mucopolysaccharidosis type IIIA (Sanfilippo syndrome). PLoS One. 2015 Mar. 25; 10(3):e0121511. doi: 10.1371/journal.pone.0121511. eCollection 2015, which is incorporated herein by reference in its entirety.

In certain embodiments, a functional hSGSH refers to an amino acid sequence of SEQ ID NO: 19, wherein the amino acid sequence comprises an exogenous leader peptide and a mature hSGSH coding sequence. In certain embodiments, the mature hSGSH further comprises stabilizing amino acid residue changes and or substitutions at A482Y and E488V, e.g., SEQ ID NO: 23. In certain embodiments, a functional hSGSH refers to an amino acid sequence of SEQ ID NO: 25, wherein the functional hSGSH comprises an exogenous leader peptide and a mature hSGSH comprising stabilizing amino acid changes A482Y and E488V.

In certain embodiments, a functional hSGSH refers to an amino acid sequence of SEQ ID NO: 21, wherein the amino acid sequence comprises an exogenous leader peptide, a mature hSGSH, and an vIGF2 peptide. In certain embodiments, a functional hSGSH refers to an amino acid sequence of SEQ ID NO: 27, wherein the amino acid sequence comprises an exogenous leader peptide, a mature hSGSH comprising stabilizing amino acid changes A482Y and E488V, and an vIGF2 peptide.

A variety of assays exist for measuring SGSH expression and activity levels by conventional methods. See, e.g., ncbi_nlm_nih_gov/gtr/all/tests/?term=C0086647[DISCUI]&filter=method:12;testtype:clinic al; Karpova E A et al, A fluorimetric enzyme assay for the diagnosis of Sanfilippo disease type A (MPS IIIA). J Inherit Metab Dis. 1996; 19(3):278-85; Tardieu M et al, Intracerebral administration of adeno-associated viral vector serotype rh.10 carrying human SGSH and SUMF1 cDNAs in children with mucopolysaccharidosis type IIIA disease: results of a phase I/II trial. Hum Gene Ther. 2014 June; 25(6):506-16. doi: 10.1089/hum.2013.238. Epub 2014 May 5; Whyte L S et al, Variables influencing fluorimetric N-sulfoglucosamine sulfohydrolase (SGSH) activity measurement in brain homogenates. Mol Genet Metab Rep. 2015 Oct. 22; 5:60-62. doi: 10.1016/j.ymgmr.2015.10.005. eCollection 2015 December; Hopwood J J et al. Diagnosis of Sanfilippo type A syndrome by estimation of sulfamidase activity using a radiolabelled tetrasaccharide substrate. Clin Chim Acta. 1982 Aug. 18; 123(3):241-50; each of which is incorporated by reference herein in its entirety.

### Nucleic Acid

In one aspect, a nucleic acid sequence which encodes a functional SGSH protein is provided. In one embodiment, the nucleic acid sequence is an engineered coding sequence, wherein the functional hSGSH coding sequence comprises a signal peptide sequence and a mature hSGSH coding sequence, wherein the mature hSGSH coding has the nucleic acid sequence of sequence of SEQ ID NO: 16 or a nucleic acid sequence which is (a) at least 85% identical thereto which encodes SEQ ID NO: 23; or (b) at least 99% identical thereto which encodes SEQ ID NO: 23. In certain embodiments, the mature hSGSH coding sequence 99% identical to SEQ ID NO: 16 is SEQ ID NO: 22 which encodes SEQ ID NO: 23 (hSGSH.A482Y.E488V). In certain embodiments, the mature hSGSH coding sequence is SEQ ID NO: 16. In certain embodiments, the mature hSGSH coding sequence which is at least about 85% identical to SEQ ID NO: 16, which encodes SEQ ID NO: 23.

In one aspect, the SGSH coding sequence is an engineered sequence. In one embodiment, the engineered sequence is useful to improve production, transcription, expression or safety in a subject. In another embodiment, the engineered sequence is useful to increase efficacy of the resulting therapeutic compositions or treatment. In a further embodiment, the engineered sequence is useful to increase the efficacy of the functional SGSH protein being expressed, but may also permit a lower dose of a therapeutic reagent that delivers the functional protein to increase safety. In certain embodiments, the hSGSH coding sequence is engineered and encodes for a functional SGSH protein comprising stabilizing amino acid changes A482Y and E488V.

In certain embodiments, the functional hSGSH coding sequence comprises a signal peptide sequence which is located at the 5′ of the mature hSGSH coding sequence. In certain embodiments, the signal peptide is at the amino-terminus (N-terminus) of the mature hSGSH. In certain embodiments, the functional hSGSH coding sequence comprises a signal peptide sequence which is a native signal peptide sequence. In certain embodiments, the native signal peptide comprises nucleic acid sequence of SEQ ID NO: 31, or a sequence at least about 95% identical thereto which encodes an amino acid sequence of SEQ ID NO: 32.

In certain embodiments, the functional hSGSH coding sequence comprises a signal peptide sequence which is an exogenous signal peptide sequence. In certain embodiments, the exogenous signal peptide is a BIP signal peptide. In certain embodiments, the exogenous signal peptide which is BIP peptide comprises nucleic acid sequence of SEQ ID NO: 29, or a sequence at least about 95% identical thereto which encodes an amino acid sequence of SEQ ID NO: 30.

In certain embodiments, the functional hSGSH coding sequence further comprises an vIGF2 peptide connected via a linker. In certain embodiments, the vIGF2 peptide comprises nucleic acid sequence of SEQ ID NO: 33, or a sequence at least about 95% identical thereto which encodes SEQ ID NO: 34. In certain embodiments, the vIGF2 peptide is connected via a linker at the carboxy-terminus (C-terminus) of the mature hSGSH. In certain embodiments, the vIGF2 peptide coding sequence comprising a linker is located at the 3′ of the mature hSGSH coding sequence.

In one aspect, the functional hSGSH coding sequence is an engineered sequence comprising, 5′ to 3′, a signal peptide coding sequence and a mature hSGSH coding sequence. In some embodiments, the functional hSGSH coding sequence is an engineered sequence comprising, 5′ to 3′, a signal peptide coding sequence and a mature hSGSH coding sequence comprising stabilizing amino acid changes A48Y and E488V. In another aspect, the functional hSGSH coding sequence is an engineered sequence comprising, 5′ to 3′, a signal peptide coding sequence, a mature hSGSH coding sequence, and a vIGF2 coding sequence comprising a linker. In some embodiments, the functional hSGSH coding sequence is an engineered sequence comprising, 5′ to 3′, a signal peptide coding sequence, a mature hSGSH coding sequence comprising stabilizing amino acid changes A48Y and E488V, and a vIGF2 coding sequence comprising a linker.

In one embodiment, provided is an engineered nucleic acid sequence comprising a sequence of SEQ ID NO: 35, wherein the sequence comprises native signal sequence and encodes a functional hSGSH (hSGSH; SEQ ID NO: 36). In one embodiment, provided herein is an engineered nucleic acid sequence of SEQ ID NO: 35, or a nucleic acid sequence at least about 99% identical thereto, encoding a functional hSGSH. In another embodiment, the hSGSH coding sequence is at least about 95%, at least about 96%, at least about 97%, at least about 98%, at least about 99% identity to SEQ ID NO: 35, wherein the sequence encodes a functional hSGSH.

In one embodiment, provided is an engineered nucleic acid sequence comprising a sequence of SEQ ID NO: 18, wherein the sequence comprises BIP signal sequence and encodes a functional hSGSH (BIP.hSGSH; SEQ ID NO: 19). In one embodiment, provided herein is an engineered nucleic acid sequence of SEQ ID NO: 18, or a nucleic acid sequence at least about 99% identical thereto, encoding a functional hSGSH. In another embodiment, the SGSH coding sequence is at least about 95%, at least about 96%, at least about 97%, at least about 98%, at least about 99% identity to SEQ ID NO: 18, wherein the sequence encodes a functional hSGSH.

In one embodiment, provided is an engineered nucleic acid sequence comprising a sequence of SEQ ID NO: 20, wherein the sequence comprises BIP signal sequence and encodes a functional hSGSH which is a fusion hSGSH (BIP.hSGSH.vIGF2; SEQ ID NO: 21). In one embodiment, provided herein is an engineered nucleic acid sequence of SEQ ID NO: 20, or a nucleic acid sequence at least about 99% identical thereto, encoding a functional hSGSH. In another embodiment, the SGSH coding sequence is at least about 95%, at least about 96%, at least about 97%, at least about 98%, at least about 99% identity to SEQ ID NO: 20, wherein the sequence encodes a functional hSGSH.

In one embodiment, provided is an engineered nucleic acid sequence comprising a sequence of SEQ ID NO: 24, wherein the sequence comprises BIP signal sequence and encodes a functional hSGSH comprising stabilizing amino acid changes A482Y and E488V (BIP.hSGSH.A488Y.E488V; SEQ ID NO: 25). In one embodiment, provided herein is an engineered nucleic acid sequence of SEQ ID NO: 24, or a nucleic acid sequence at least about 99% identical thereto, encoding a functional hSGSH. In another embodiment, the SGSH coding sequence is at least about 95%, at least about 96%, at least about 97%, at least about 98%, at least about 99% identity to SEQ ID NO: 24, wherein the sequence encodes a functional hSGSH.

In one embodiment, provided is an engineered nucleic acid sequence comprising a sequence of SEQ ID NO: 26, wherein the sequence comprises BIP signal sequence and encodes a functional hSGSH which is a fusion hSGSH comprising stabilizing amino acid changes A482Y and E488V (BIP.hSGSH.A488Y.E488V.vIGF2; SEQ ID NO: 27). In one embodiment, provided herein is an engineered nucleic acid sequence of SEQ ID NO: 26, or a nucleic acid sequence at least about 99% identical thereto, encoding a functional hSGSH. In another embodiment, the SGSH coding sequence is at least about 95%, at least about 96%, at least about 97%, at least about 98%, at least about 99% identity to SEQ ID NO: 26, wherein the sequence encodes a functional hSGSH.

A “nucleic acid”, as described herein, can be RNA, DNA, or a modification thereof, and can be single or double stranded, and can be selected, for example, from a group including nucleic acid encoding a protein of interest, oligonucleotides, nucleic acid analogues, for example peptide-nucleic acid (PNA), pseudocomplementary PNA (pc-PNA), locked nucleic acid (LNA) etc. A nucleotide refers to a ribonucleotide, deoxynucleotide or a modified form of either type of nucleotide (e.g., a peptide nucleic acid oligomer). The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

In certain embodiments, the nucleic acid molecules encoding a functional human SGSH (hSGSH), and other constructs encompassed by the present invention and useful in generating expression cassettes and vector genomes may be engineered for expression in yeast cells, insect cells or mammalian cells, such as human cells. Methods are known and have been described previously (e.g., WO 96/09378). A sequence is considered engineered if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g., GeneArt, GenScript, Life Technologies, Eurofins).

By “engineered” is meant that the nucleic acid sequences encoding a functional SGSH protein described herein are assembled and placed into any suitable genetic element, e.g., naked DNA, phage, transposon, cosmid, episome, etc., which transfers the SGSH sequences carried thereon to a host cell, e.g., for generating non-viral delivery systems (e.g., RNA-based systems, naked DNA, or the like), or for generating viral vectors in a packaging host cell, and/or for delivery to a host cells in a subject. In one embodiment, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “Clustal Omega” “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “Clustal Omega”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

As used herein, “a desired function” refers to an SGSH enzyme activity at least about 20%, about 25%, about 30%, about 35%, about 40%, about 45%, about 50%, about 55%, about 60%, about 65%, about 70%, about 75%, about 80%, about 85%, about 90%, about 95%, or about 100% of a healthy control.

As used herein, the phrases “ameliorate a symptom”, “improve a symptom” or any grammatical variants thereof, refer to reversal of an MPS IIIA-related symptoms, showdown or prevention of progression of an MPS IIIA-related symptoms. In one embodiment, the amelioration or improvement refers to the total number of symptoms in a patient after administration of the described composition(s) or use of the described method, which is reduced by about 5%, about 10%, about 20%, about 30%, about 40%, about 50%, about 60%, about 70%, about 80%, about 90%, about 95% compared to that before the administration or use. In another embodiment, the amelioration or improvement refers to the severity or progression of a symptom after administration of the described composition(s) or use of the described method, which is reduced by about 5%, about 10%, about 20%, about 30%, about 40%, about 50%, about 60%, about 70%, about 80%, about 90%, about 95% compared to that before the administration or use.

It should be understood that the compositions in the SGSH functional protein and SGSH coding sequence described herein are intended to be applied to other compositions, regiments, aspects, embodiments and methods described across the Specification.

### Expression Cassette and Vector Genome

A gene therapy vector is provided herein which comprises an expression cassette comprising an engineered nucleic acid sequence comprising coding sequences for hSGSH operably linked to regulatory sequences which direct expression thereof. In certain embodiments, the expression cassette comprises

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in cis or trans with nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′ UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette for generating a viral vector contains the coding sequence for the gene product described herein flanked by packaging signals of the viral genome and other expression control sequences such as those described herein. In certain embodiments, a vector genome may contain two or more expression cassettes.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g., a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

In one embodiment, the regulatory sequence comprises a promoter. In one embodiment, the promoter is a chicken β-actin (CB) promoter. In a further embodiment, the promoter is CB7 promoter which is a hybrid of a cytomegalovirus immediate-early (CMV IE) enhancer and the chicken β-actin promoter (a CB7 promoter). In certain embodiments, the CB7 promoter comprises nucleic acid sequence of SEQ ID NO: 44. In another embodiment, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kügler S et al, Human synapsin 1 gene promoter confers highly neuron-specific long-term transgene expression from an adenoviral vector in the adult rat brain depending on the transduced area. Gene Ther. 2003 February; 10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 February; 145(2):613-9. Epub 2003 Oct. 16), or a CB6 promoter (see, e.g., Large-Scale Production of Adeno-Associated Viral Vector Serotype-9 Carrying the Human Survival Motor Neuron Gene, Mol Biotechnol. 2016 January; 58(1):30-6. doi: 10.1007/s12033-015-9899-5).

In certain embodiments, an additional or alternative promoter sequence may be included as part of the expression control sequences (regulatory sequences), e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be utilized in the vectors described herein. The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

In one embodiment, the expression cassette is designed for expression and secretion in a human subject. In one embodiment, the expression cassette is designed for expression in the central nervous system (CNS), including the cerebral spinal fluid and brain. In a further embodiment, the expression cassette is useful for expression in both the CNS and in the liver. Suitable promoters may be selected, including but not limited to a constitutive promoter, a tissue-specific promoter or an inducible/regulatory promoter. Example of a constitutive promoter is chicken beta-actin promoter. A variety of chicken beta-actin promoters have been described alone, or in combination with various enhancer elements (e.g., CB7 is a chicken beta-actin promoter with cytomegalovirus enhancer elements; a CAG promoter, which includes the promoter, the first exon and first intron of chicken beta actin, and the splice acceptor of the rabbit beta-globin gene; a CBh promoter, S J Gray et al, Hu Gene Ther, 2011 September; 22(9): 1143-1153). Examples of promoters that are tissue-specific are well known for liver (albumin, Miyatake et al., (1997) J. Virol., 71:5124 32; hepatitis B virus core promoter, Sandig et al., (1996) Gene Ther., 3:1002 9; alpha fetoprotein (AFP), Arbuthnot et al., (1996) Hum. Gene Ther., 7:1503 14), neuron (such as neuron specific enolase (NSE) promoter, Andersen et al., (1993) Cell. Mol. Neurobiol., 13:503 15; neurofilament light chain gene, Piccioli et al., (1991) Proc. Natl. Acad. Sci. USA, 88:5611 5; and the neuron-specific vgf gene, Piccioli et al., (1995) Neuron, 15:373 84), and other tissues. Alternatively, a regulatable promoter may be selected. See, e.g., WO 2011/126808B2, incorporated by reference herein.

In addition to a promoter, a vector may contain one or more other appropriate transcription initiation sequences, transcription termination sequences, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product.

In one embodiment, the regulatory sequence further comprises an enhancer. In one embodiment, the regulatory sequence comprises one enhancer. In another embodiment, the regulatory sequence contains two or more expression enhancers. These enhancers may be the same or may be different. For example, an enhancer may include an Alpha mic/bik enhancer or a CMV enhancer. This enhancer may be present in two copies which are located adjacent to one another. Alternatively, the dual copies of the enhancer may be separated by one or more sequences.

In one embodiment, the regulatory sequence further comprises an intron. In a further embodiment, the intron is a chicken beta-actin intron. In certain embodiments, the chicken beta-actin intron comprises nucleic acid sequence of SEQ ID NO: 45. In certain embodiments, the intron is a chimeric intron (CI)—a hybrid intron consisting of a human beta-globin splice donor and immunoglobulin G (IgG) splice acceptor elements. Other suitable introns include those known in the art, e.g., such as are described in WO 2011/126808. Other suitable introns include those known in the art may by a human 0-globulin intron, and/or a commercially available Promega® intron, and those described in WO 2011/126808.

In one embodiment, the regulatory sequence further comprises a Polyadenylation signal (polyA). Examples of suitable polyA sequences include, e.g., Rabbit globin poly A, SV40, SV50, bovine growth hormone (bGH), human growth hormone, and synthetic polyAs. Optionally, one or more sequences may be selected to stabilize mRNA. In certain embodiments, the polyA is a rabbit beta globin poly A (rabbit globin polya or rBG). See, e.g., WO 2014/151341. In certain embodiments, the rabbit beta globin polyA comprises nucleic acid sequence of SEQ ID NO: 46. In certain embodiments, a human growth hormone (hGH) polyadenylation sequence, an SV40 polyA, or a synthetic polyA may be included in an expression cassette.

In certain embodiments, the expression cassettes provided may include one or more expression enhancers such as post-transcriptional regulatory element from hepatitis viruses of woodchuck (WPRE), human (HPRE), ground squirrel (GPRE) or arctic ground squirrel (AGSPRE); or a synthetic post-transcriptional regulatory element. These expression-enhancing elements are particularly advantageous when placed in a 3′ UTR and can significantly increase mRNA stability and/or protein yield. In certain embodiments, the expressions cassettes provided include a regulator sequence that is a woodchuck hepatitis virus posttranscriptional regulatory element (WPRE) or a variant thereof. Suitable WPRE sequences are provided in the vector genomes described herein and are known in the art (e.g., such as those are described in U.S. Pat. Nos. 6,136,597, 6,287,814, and 7,419,829, which are incorporated by reference). In certain embodiments, the WPRE is a variant that has been mutated to eliminate expression of the woodchuck hepatitis B virus X (WHX) protein, including, for example, mutations in the start codon of the WHX gene. See also, Kingsman S. M., Mitrophanous K., & Olsen J. C. (2005), Potential Oncogene Activity of the Woodchuck Hepatitis Post-Transcriptional Regulatory Element (Wpre).” Gene Ther. 12(1):3-4; and Zanta-Boussif M. A., Charrier S., Brice-Ouzet A., Martin S., Opolon P., Thrasher A. J., Hope T.J., & Galy A. (2009), Validation of a Mutated Pre Sequence Allowing High and Sustained Transgene Expression While Abrogating Whv-X Protein Synthesis: Application to the Gene Therapy of Was, Gene Ther. 16(5):605-19, both of which are incorporated herein by reference in its entirety. In other embodiments, enhancers are selected from a non-viral source. In certain embodiments, no WPRE sequence is present. IN certain embodiments, the WPRE comprises nucleic acid sequence of SEQ ID NO: 43.

In certain embodiments, the expression cassette comprises a hSGSH coding sequence and may include other regulatory sequences therefor. The regulatory sequences necessary are operably linked to the hSGSH coding sequence in a manner which permits its transcription, translation and/or expression in target cell.

In certain embodiment, the target cell may be a central nervous system cell. In certain embodiments, the target cell is one or more of an excitatory neuron, an inhibitory neuron, a glial cell, a cortex cell, a frontal cortex cell, a cerebral cortex cell, a spinal cord cell. In certain embodiments, the target cell is a peripheral nervous system (PNS) cell, for example a retina cell. Other cells other than those from nervous system may also be chosen as a target cell, such as a monocyte, a B lymphocyte, a T lymphocyte, a NK cell, a lymph node cell, a tonsil cell, a bone marrow mesenchymal cell, a stem cell, a bone marrow stem cell, a heart cell, an epithelium cell, a esophagus cell, a stomach cell, a fetal cut cell, a colon cell, a rectum cell, a liver cell, a kidney cell, a lung cell, a salivary gland cell, a thyroid cell, an adrenal cell, a breast cell, a pancreas cell, an islet of Langerhans cell, a gallbladder cell, a prostate cell, a urinary bladder cell, a skin cell, a uterus cell, a cervix cell, a testis cell, or any other cell which expresses a functional SGSH enzyme in a subject without MPSIIIA.

In certain embodiments, the expression cassette comprises CB7 promoter—optionally chicken beta actin intron sequence—hSGSH coding sequence—rabbit beta-globin polyA. In certain embodiments, the expression cassette comprises CB7 promoter—optionally chicken beta actin intron sequence—hSGSH coding sequence comprising BIP exogenous leader peptide—rabbit beta-globin polyA. In certain embodiments, the expression cassette comprises CB7 promoter—optionally chicken beta actin intron sequence—hSGSH coding sequence comprising BIP exogenous leader peptide at 5′ to hSGSH coding sequence and vIGF2 peptide at 3′ to hSGSH coding sequence—rabbit beta-globin polyA. In certain embodiments, the expression cassette comprises CB7 promoter—optionally chicken beta actin intron sequence—hSGSH coding sequence comprising BIP exogenous leader peptide at 5′ to hSGSH coding sequence and vIGF2 peptide at 3′ to hSGSH coding sequence—optionally WPRE—rabbit beta-globin polyA. In certain embodiments, the expression cassette comprises CB7 promoter—optionally chicken beta actin intron sequence—hSGSH coding sequence comprising BIP exogenous leader peptide at 5′ to hSGSH coding sequence and vIGF2 peptide at 3′ to hSGSH coding sequence, optionally wherein hSGSH comprises stabilizing amino acid changes A482Y and E488V (hSGSH.A482Y-E488V)—optionally WPRE—rabbit beta-globin polyA.

In certain embodiments, the expression cassette comprises CB7 promoter (SEQ ID NO: 44)—optionally chicken beta actin intron sequence (SEQ ID NO: 45)—hSGSH coding sequence comprising BIP exogenous leader peptide at 5′ to hSGSH coding sequence and vIGF2 peptide at 3′ to hSGSH coding sequence—optionally WPRE (SEQ ID NO: 43)—rabbit beta-globin polyA (SEQ ID NO: 46). In certain embodiments, the expression cassette comprises CB7 promoter (SEQ ID NO: 44)—optionally chicken beta actin intron sequence (SEQ ID NO: 45)—hSGSH (optionally hSGSH.A482Y-E488V) coding sequence comprising BIP exogenous leader peptide at 5′ to hSGSH coding sequence and vIGF2 peptide at 3′ to hSGSH coding sequence—optionally WPRE (SEQ ID NO: 43)—rabbit beta-globin polyA (SEQ ID NO: 46). In certain embodiments, the expression cassette comprises nucleic acid sequence of SEQ ID NO: 8.

In certain embodiments, the expression cassette comprises nucleic acid sequence of SEQ ID NO: 4 (CB7.CI.BIP.hSGSHcov1-A482Y_E488V.vIGF2.rBG) or a sequence at least 95%, at least 96%, at least 97%, at least 98%, at least 99 to at least 100% identical thereto. In certain embodiments, the expression cassette comprises nucleic acid sequence of SEQ ID NO: 11 (CB7.CI.hSGSHcoV1.rBG) or a sequence at least 95%, at least 96%, at least 97%, at least 98%, at least 99 to at least 100% identical thereto. In certain embodiments, the expression cassette comprises nucleic acid sequence of SEQ ID NO: 8 (CB7.CI.BIP.hSGSHcov1-A482Y_E488V.vIGF2.WPRE.rBG) or a sequence at least 95%, at least 96%, at least 97%, at least 98%, at least 99 to at least 100% identical thereto.

It should be understood that the compositions in the expression cassette described herein are intended to be applied to other compositions, regiments, aspects, embodiments and methods described across the Specification.

miRNA

In certain embodiments, in addition to the hSGSH coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

As used herein, an “miRNA target sequence” is a sequence located on the DNA positive strand (5′ to 3′) and is at least partially complementary to a miRNA sequence, including the miRNA seed sequence. The miRNA target sequence is exogenous to the untranslated region of the encoded transgene product and is designed to be specifically targeted by miRNA in cells in which repression of transgene expression is desired. The term “miR183 cluster target sequence” refers to a target sequence that responds to one or members of the miR183 cluster (alternatively termed family), including miRs-183, and -182 (as described by Dambal, S. et al. Nucleic Acids Res 43:7173-7188, 2015, which is incorporated herein by reference). Without wishing to be bound by theory, the messenger RNA (mRNA) for the transgene (encoding the gene product) is present in a cell type to which the expression cassette containing the miRNA is delivered, such that specific binding of the miRNA to the 3′ UTR miRNA target sequences results in mRNA silencing and cleavage, thereby reducing or eliminating transgene expression only in the cells that express the miRNA.

Typically, the miRNA target sequence is at least 7 nucleotides to about 28 nucleotides in length, at least 8 nucleotides to about 28 nucleotides in length, 7 nucleotides to 28 nucleotides, 8 nucleotides to 18 nucleotides, 12 nucleotides to 28 nucleotides in length, about 20 to about 26 nucleotides, about 22 nucleotides, about 24 nucleotides, or about 26 nucleotides, and contains at least one consecutive region (e.g., 7 or 8 nucleotides) which is complementary to the miRNA seed sequence. In certain embodiments, the target sequence comprises a sequence with exact complementarity (100%) or partial complementarity to the miRNA seed sequence with some mismatches. In certain embodiments, the target sequence comprises at least 7 to 8 nucleotides which are 100% complementary to the miRNA seed sequence. In certain embodiments, the target sequence consists of a sequence which is 100% complementary to the miRNA seed sequence. In certain embodiments, the target sequence contains multiple copies (e.g., two, three, four or more copies) of the sequence which is 100% complementary to the seed sequence. In certain embodiments, the region of 100% complementarity comprises at least 30% of the length of the target sequence. In certain embodiments, the remainder of the target sequence has at least about 80% to about 99% complementarity to the miRNA. In certain embodiments, in an expression cassette containing a DNA positive strand, the miRNA target sequence is the reverse complement of the miRNA.

In certain embodiments, the miRNA target sequence for the at least first and/or at least second miRNA target sequence for the expression cassette mRNA or DNA positive strand is selected from (i) AGTGAATTCTACCAGTGCCATA (miR183, SEQ ID NO: 28); (ii) AGTGTGAGTTCTACCATTGCCAAA (miR182, SEQ ID NO: 47).

In certain embodiments, the vector genome or expression cassette contains at least one miRNA target sequence that is a miR-183 target sequence. In certain embodiments, the vector genome or expression cassette contains an miR-183 target sequence that includes AGTGAATTCTACCAGTGCCATA (SEQ ID NO: 28), where the sequence complementary to the miR-183 seed sequence is underlined. In certain embodiments, the vector genome or expression cassette contains more than one copy (e.g., two or three copies) of a sequence that is 100% complementary to the miR-183 seed sequence. In certain embodiments, the vector genome or expression cassette contains 4 copies of a sequence that is 100% complementary to the miR-183 seed sequence. In certain embodiments, a miR-183 target sequence is about 7 nucleotides to about 28 nucleotides in length and includes at least one region that is at least 100% complementary to the miR-183 seed sequence. In certain embodiments, a miR-183 target sequence contains a sequence with partial complementarity to SEQ ID NO: 28 and, thus, when aligned to SEQ ID NO: 28, there are one or more mismatches. In certain embodiments, a miR-183 target sequence comprises a sequence having at least 1, 2, 3, 4, 5, 6, 7, 8, 9, or 10 mismatches when aligned to SEQ ID NO: 28, where the mismatches may be non-contiguous. In certain embodiments, a miR-183 target sequence includes a region of 100% complementarity which also comprises at least 30% of the length of the miR-183 target sequence. In certain embodiments, the region of 100% complementarity includes a sequence with 100% complementarity to the miR-183 seed sequence. In certain embodiments, the remainder of a miR-183 target sequence has at least about 80% to about 99% complementarity to miR-183. In certain embodiments, the expression cassette or vector genome includes a miR-183 target sequence that comprises a truncated SEQ ID NO: 28, i.e., a sequence that lacks at least 1, 2, 3, 4, 5, 6, 7, 8, 9, or 10 nucleotides at either or both the 5′ or 3′ ends of SEQ ID NO: 28. In certain embodiments, the expression cassette or vector genome comprises a transgene and one miR-183 target sequence. In yet other embodiments, the expression cassette or vector genome comprises at least two, three or four miR-183 target sequences.

In certain embodiments, the vector genome or expression cassette contains at least one miRNA target sequence that is a miR-182 target sequence. In certain embodiments, the vector genome or expression cassette contains an miR-182 target sequence that includes AGTGTGAGTTCTACCATTGCCAAA (SEQ ID NO: 47). In certain embodiments, the vector genome or expression cassette contains more than one copy (e.g., two or three copies) of a sequence that is 100% complementary to the miR-182 seed sequence. In certain embodiments, a miR-182 target sequence is about 7 nucleotides to about 28 nucleotides in length and includes at least one region that is at least 100% complementary to the miR-182 seed sequence. In certain embodiments, a miR-182 target sequence contains a sequence with partial complementarity to SEQ ID NO: 47 and, thus, when aligned to SEQ ID NO: 47, there are one or more mismatches. In certain embodiments, a miR-183 target sequence comprises a sequence having at least 1, 2, 3, 4, 5, 6, 7, 8, 9, or 10 mismatches when aligned to SEQ ID NO: 47, where the mismatches may be non-contiguous. In certain embodiments, a miR-182 target sequence includes a region of 100% complementarity which also comprises at least 30% of the length of the miR-182 target sequence. In certain embodiments, the region of 100% complementarity includes a sequence with 100% complementarity to the miR-182 seed sequence. In certain embodiments, the remainder of a miR-182 target sequence has at least about 80% to about 99% complementarity to miR-182. In certain embodiments, the expression cassette or vector genome includes a miR-182 target sequence that comprises a truncated SEQ ID NO: 47, i.e., a sequence that lacks at least 1, 2, 3, 4, 5, 6, 7, 8, 9, or 10 nucleotides at either or both the 5′ or 3′ ends of SEQ ID NO: 47. In certain embodiments, the expression cassette or vector genome comprises a transgene and one miR-182 target sequence. In yet other embodiments, the expression cassette or vector genome comprises at least two, three or four miR-182 target sequences.

The term “tandem repeats” is used herein to refer to the presence of two or more consecutive miRNA target sequences. These miRNA target sequences may be continuous, i.e., located directly after one another such that the 3′ end of one is directly upstream of the 5′ end of the next with no intervening sequences, or vice versa. In another embodiment, two or more of the miRNA target sequences are separated by a short spacer sequence.

As used herein, as “spacer” is any selected nucleic acid sequence, e.g., of 1, 2, 3, 4, 5, 6, 7, 8, 9 or 10 nucleotides in length which is located between two or more consecutive miRNA target sequences. In certain embodiments, the spacer is 1 to 8 nucleotides in length, 2 to 7 nucleotides in length, 3 to 6 nucleotides in length, four nucleotides in length, 4 to 9 nucleotides, 3 to 7 nucleotides, or values which are longer. Suitably, a spacer is a non-coding sequence. In certain embodiments, the spacer may be of four (4) nucleotides. In certain embodiments, the spacer is GGAT. In certain embodiments, the spacer is six (6) nucleotides. In certain embodiments, the spacer is CACGTG or GCATGC. In certain embodiments, the spacer is independently selected from one or more of (A) GGAT; (B) CACGTG; (C) GCATGC; (D) GCGGCCGC; (E) CGAT; (F) ATCGGT; and/or (G) TCAC.

In certain embodiments, the tandem repeats contain two, three, four or more of the same miRNA target sequence. In certain embodiments, the tandem repeats contain at least two different miRNA target sequences, at least three different miRNA target sequences, or at least four different miRNA target sequences, etc. In certain embodiments, the tandem repeats may contain two or three of the same miRNA target sequence and a fourth miRNA target sequence which is different.

In certain embodiments, there may be at least two different sets of tandem repeats in the expression cassette. For example, a 3′ UTR may contain a tandem repeat immediately downstream of the transgene, UTR sequences, and two or more tandem repeats closer to the 3′ end of the UTR. In another example, the 5′ UTR may contain one, two or more miRNA target sequences. In another example the 3′ may contain tandem repeats and the 5′ UTR may contain at least one miRNA target sequence.

In certain embodiments, the expression cassette contains two, three, four or more tandem repeats which start within about 0 to 20 nucleotides of the stop codon for the transgene. In certain embodiments, the expression cassette contains two, three, four, five, six, seven, eight or more tandem repeats which start within about 0 to 20 nucleotides of the stop codon for the transgene. In other embodiments, the expression cassette contains the miRNA tandem repeats at least 100 to about 4000 nucleotides from the stop codon for the transgene. The target miRNA sequences may be selected from SEQ ID NO: 28, and/or SEQ ID NO: 47.

In certain embodiments, the vector genome further comprises at least one, at least two, at least three or preferably at least four tandem repeats of dorsal root ganglion (drg)-specific miRNA target sequences. In certain embodiments, the vector genome further comprises at least one, at least two, at least three, at least four, at least five, at least six, at least seven or preferably at least eight tandem repeats of dorsal root ganglion (drg)-specific miRNA target sequences. See, e.g., PCT/US19/67872, filed Dec. 20, 2019 and now published as WO 2020/132455. See, also, U.S. Provisional Patent Application No. 63/023,593, filed May 12, 2020; U.S. Provisional Patent Application No. 63/038,488, filed Jun. 12, 2020; U.S. Provisional Patent Application No. 63/043,562, filed Jun. 24, 2020; and U.S. Provisional Patent Application No. 63/079,299, filed Jun. 24, 2020, U.S. Provisional Patent Application No. 63/079,299, 10 filed Sep. 16, 2020, and U.S. Provisional Patent Application No. 63/152,042, filed Feb. 22, 2021, and International Patent Application No. PCT/US21/32003 which are incorporated herein by reference.

In certain embodiments, the expression cassette comprises at least eight miRNA drg de-targeting sequences. In certain embodiments, the expression cassette comprises at least eight miRNA drg de-targeting sequences comprise miR182 and miR183. In certain embodiments, the expression cassette comprises at least eight miRNA drg de-targeting sequences, wherein the at least first, at least second, at least third, and at least fourth miRNA is miR182 sequence, and at least fifth, at least sixth, at least seventh, and at least eighth miRNA is miR183 sequence. In certain embodiments, the expression cassette or the vector genome comprises at least 8 miRNA drg de-targeting sequences, wherein the at least first, at least second, at least third, and at least fourth miRNA is miR183 sequence, and at least fifth, at least sixth, at least seventh, and at least eighth miRNA is miR182 sequence.

In certain embodiments, the invention provides a nucleic acid molecule having an expression cassette which comprises an hSGSH coding sequence as defined herein, four miR182 sequences, four miR183 sequences, and other suitable regulatory sequences operably linked to the SGSH coding sequence. In certain embodiments, the expression cassette comprising an open reading frame (ORF) sequence (e.g., ORF comprising hSGSH coding sequence operably linked to regulatory control sequences), and DRG-detargeting sequences. In certain embodiments, the DRG-detargeting sequences are located 5′ to the coding sequence. In certain embodiments, the DRG-detargeting sequences are located 3′ to the coding sequence.

In certain embodiments, the regulatory sequences comprise a CB7 promoter. In certain embodiment, the regulatory sequences comprise one or more intron(s), one or more enhancer(s), and a polyA. In certain embodiments, the expression cassette comprises CB7 promoter—optionally chicken beta actin intron sequence—hSGSH coding sequence—at least four copies of miRNA182—at least 4 copies of miR183—rabbit beta-globin polyA. In certain embodiments, the expression cassette comprises CB7 promoter—optionally chicken beta actin intron sequence—hSGSH coding sequence comprising BIP exogenous leader peptide—at least four copies of miRNA182—at least 4 copies of miR183—rabbit beta-globin polyA. In certain embodiments, the expression cassette comprises CB7 promoter—optionally chicken beta actin intron sequence—hSGSH coding sequence comprising BIP exogenous leader peptide at 5′ to hSGSH coding sequence and vIGF2 peptide at 3′ to hSGSH coding sequence—at least four copies of miRNA182—at least 4 copies of miR183—rabbit beta-globin polyA. In certain embodiments, the expression cassette comprises nucleic acid sequence of SEQ ID NO: 37.

In certain embodiments, the expression cassette comprises CB7 promoter—optionally chicken beta actin intron sequence—hSGSH coding sequence which is a wild type coding sequence—at least four copies of miRNA 182—rabbit beta-globin polyA. In certain embodiments, the expression cassette comprises CB7 promoter—optionally chicken beta actin intron sequence—hSGSH coding sequence which is a wild type coding sequence—at least 4 copies of miR183—rabbit beta-globin polyA. In certain embodiments, the expression cassette comprises CB7 promoter—optionally chicken beta actin intron sequence—hSGSH coding sequence which is a wild type coding sequence—at least four copies of miRNA182—at least 4 copies of miR183—rabbit beta-globin polyA. In certain embodiments, the wild type hSGSH coding sequence comprising native signal peptide and mature hSGSH comprises SEQ ID NO: SEQ ID NO: 60.

In certain embodiments, the expression cassette refers to nucleic acid molecule of SEQ ID NOs: 2, 38, 41. In certain embodiments, the expression cassette refers to nucleic acid molecule of SEQ ID NO: 2, encoding for hSGSH, and comprising 4 tandem repeats of miRNA183 (miR183; SEQ ID NO: 28). In certain embodiments, the expression cassette refers to nucleic acid molecule of SEQ ID NO: 41, encoding for hSGSH, and comprising 4 tandem repeats of miRNA182 (miR182; SEQ ID NO: 48). In certain embodiments, the expression cassette refers to nucleic acid molecule of SEQ ID NO: 38, encoding for hSGSH, and comprising 4 tandem repeats of miRNA182 (miR182; SEQ ID NO: 48) and 4 tandem repeats of miRNA183 (miR183; SEQ ID NO: 28).

In certain embodiments, the expression cassette comprises nucleic acid sequence of SEQ ID NO: 14 (CB7.CI.hSGSHcoV1-4xmiR183.rBG) or a sequence at least 95%, at least 96%, at least 97%, at least 98%, at least 99 to at least 100% identical thereto, and/or values therebetween. In certain embodiments, the expression cassette comprises nucleic acid sequence of SEQ ID NO: 41 (CB7.CI.BIP.hSGSHcov1-A482Y_E488V.vIGF2.WPRE.4xmiR182.rBG) or a sequence at least 95%, at least 96%, at least 97%, at least 98%, at least 99 to at least 100% identical thereto, and/or values therebetween. In certain embodiments, the expression cassette comprises nucleic acid sequence of SEQ ID NO: 2 (CB7.CI.BIP.hSGSHcov1(A482Y-E488V).vIGF2.WPRE.4xmiR183.rBG) or a sequence at least 95%, at least 96%, at least 97%, at least 98%, at least 99 to at least 100% identical thereto, and/or values therebetween. In certain embodiments, the expression cassette comprises nucleic acid sequence of SEQ ID NO: 38 (CB7.CI.BIP.hSGSHcov1-A482Y_E488V.vIGF2.WPRE.4xmiR182.4xmiR183.rBG) or a sequence at least 95%, at least 96%, at least 97%, at least 98%, at least 99 to at least 100% identical thereto, and/or values therebetween.

### Vector

In one aspect, provided herein is a vector comprising an engineered nucleic acid sequence encoding a functional human N-sulfoglycosamine sulfohydrolase (hSGSH) and a regulatory sequence which direct expression thereof in a target cell, wherein the hSGSH coding sequence comprises a signal peptide sequence and a mature hSGSH coding sequence. In one embodiment, the mature hSGSH coding sequence is SEQ ID NO: 16, which encodes amino acid sequence SEQ ID NO: 17. In certain embodiments, the mature hSGSH coding sequence is a sequence at least about 85% identical to SEQ ID NO: 16 and encoding amino acid sequence of SEQ ID NO: 17. In certain embodiments, the mature hSGSH coding sequence is a sequence at least about 85% identical to SEQ ID NO: 16 and encoding amino acid sequence of SEQ ID NO: 23. In a further embodiment, the mature hSGSH coding sequence is at least 99% identical to SEQ ID NO: 16 which encodes amino acid sequence of SEQ ID NO: 23. In yet a further embodiment, the mature hSGSH coding sequence is SEQ ID NO: 22 or a sequence at least about 99% identical thereto which encodes amino acid sequence of SEQ ID NO: 23.

In certain embodiments, the vector comprises hSGSH coding sequence comprising a native leader peptide sequence and a mature hSGSH coding sequence, wherein the hSGSH mature coding sequence is selected from SEQ ID NO: 16 or a sequence at least 95% identical thereto, or SEQ ID NO: 22 or a sequence at least about 95% identical thereto. In certain embodiments, the vector comprises hSGSH coding sequence comprising an exogenous leader peptide sequence and a mature hSGSH coding sequence, wherein the hSGSH mature coding sequence is selected from SEQ ID NO: 16 or a sequence at least 95% identical thereto, or SEQ ID NO: 22 or a sequence at least about 95% identical thereto.

In certain embodiments, the vector comprises hSGSH coding sequence comprising a native leader peptide sequence and a mature hSGSH coding sequence, wherein hSGSH coding sequence is SEQ ID NO: 35 or a sequence at least 95% identical thereto. In certain embodiments, the vector comprises hSGSH coding sequence comprising an exogenous leader peptide sequence and a mature hSGSH coding sequence, wherein hSGSH coding sequence is SEQ ID NO: 18 or a sequence at least 95% identical thereto. In certain embodiments, the vector comprises hSGSH coding sequence comprising an exogenous leader peptide sequence and a mature hSGSH coding sequence comprising stabilizing amino acid residue changes A482Y and E488V, wherein hSGSH coding sequence is SEQ ID NO: 24 or a sequence at least 95% identical thereto.

In further embodiments, the vector comprises hSGSH coding sequence comprising an exogenous leader peptide sequence, a mature hSGSH coding sequence, a linker sequence and a vIGF2 peptide sequence, wherein hSGSH coding sequence is SEQ ID NO: 20 or a sequence at least 95% identical thereto. In further embodiments, the vector comprises hSGSH coding sequence comprising an exogenous leader peptide sequence, a mature hSGSH coding sequence comprising stabilizing amino acid residue changes A482Y and E488V, a linker sequence and a vIGF2 peptide sequence, wherein hSGSH coding sequence is SEQ ID NO: 26 or a sequence at least 95% identical thereto.

A “vector” as used herein is a biological or chemical moiety comprising a nucleic acid sequence which can be introduced into an appropriate target cell for replication or expression of said nucleic acid sequence. Examples of a vector includes but not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule into which an exogenous or heterologous or engineered nucleic acid encoding a functional SGSH may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization or quantification of the vectors are available to one of skill in the art.

In one embodiment, the vector is a non-viral plasmid that comprises an expression cassette described thereof, e.g., “naked DNA”, “naked plasmid DNA”, naked RNA, and mRNA; coupled with various compositions and nano particles, including, e.g., micelles, liposomes, cationic lipid—nucleic acid compositions, poly-glycan compositions and other polymers, lipid and/or cholesterol-based—nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiments, the vector described herein is a “replication-defective virus” or a “viral vector” which refers to a synthetic or artificial viral particle in which an expression cassette containing a nucleic acid sequence encoding SGSH is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the nucleic acid sequence encoding SGSH flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, a recombinant virus vector is an adeno-associated virus (AAV), an adenovirus, a bocavirus, a hybrid AAV/bocavirus, a herpes simplex virus or a lentivirus.

As used herein, the term “host cell” may refer to the packaging cell line in which a vector (e.g., a recombinant AAV) is produced. A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

As used herein, the term “target cell” refers to any target cell in which expression of the functional SGSH is desired. In certain embodiments, the term “target cell” is intended to reference the cells of the subject being treated for MPS IIIA. Examples of target cells may include, but are not limited to, a liver cell, a kidney cell, a cell of the central nervous system, a neuron, a glial cell, and a stem cell. In certain embodiments, the vector is delivered to a target cell ex vivo. In certain embodiments, the vector is delivered to the target cell in vivo.

It should be understood that the compositions in the vector described herein are intended to be applied to other compositions, regiments, aspects, embodiments and methods described across the Specification.

Recombinant Adeno-Associated Virus (rAAV)

Provided herein is a recombinant adeno-associated virus (rAAV) useful for treating Mucopolysaccharidosis III A (MPS IIIA). The rAAV comprises (a) an AAV capsid; and (b) a vector genome packaged in the AAV capsid of (a). Suitably, the AAV capsid selected targets the cells to be treated. In certain embodiments, the capsid is from Clade F. However, in certain embodiments, another AAV capsid source may be selected, i.e., Clade A. In certain embodiments, the AAV capsid is AAVhu68 capsid. In certain embodiments, the AAV capsid is AAVrh91 capsid. In certain embodiments, the AAV capsid is AAVhu95 capsid. In certain embodiments, the AAV capsid is AAVhu96 capsid. The vector genome comprises an AAV 5′ inverted terminal repeat (ITR), an engineered nucleic acid sequence encoding a functional hSGSH as described herein, a regulatory sequence which direct expression of functional hSGSH in a target cell, and an AAV 3′ ITR.

As used herein, the term “vector genome” refers to a nucleic acid molecule which is packaged in a viral capsid, for example, an AAV capsid, and is capable of being delivered to a host cell or a cell in a patient. In certain embodiments, the vector genome comprises terminal repeat sequences (e.g., AAV inverted terminal repeat sequences (ITRs) necessary for packaging the vector genome into the capsid at the extreme 5′ and 3′ end and containing therebetween an expression cassette comprising the MECP2 gene as described herein operably linked to sequences which direct expression thereof.

The AAV sequences of the vector typically comprise the cis-acting 5′ and 3′ inverted terminal repeat (ITR) sequences (See, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. A shortened version of the 5′ ITR, termed ΔITR, has been described in which the D-sequence and terminal resolution site (trs) are deleted. In certain embodiments, the vector genome (e.g., of a plasmid) includes a shortened AAV2 ITR of 130 base pairs, wherein the external A elements is deleted. The shortened ITR may revert back to the wild-type length of 145 base pairs during vector DNA amplification using the internal A element as a template and packaging into the capsid to form the viral particle. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In one aspect, the rAAV is for use in the treatment of Mucopolysaccharidosis III A (MPS IIIA). In one embodiment, the rAAV comprises a vector genome comprising 5′ AAV ITR, an expression cassette, and 3′ AAV ITR, wherein the expression cassette comprises an engineered nucleic acid sequence encoding for a functional hSGSH, wherein the functional hSGSH coding sequence comprises a signal peptide sequence and a mature hSGSH coding sequence, wherein the mature hSGSH coding has the nucleic acid sequence of sequence of SEQ ID NO: 16 or a nucleic acid sequence which is (a) at least 85% identical thereto which encodes SEQ ID NO: 23; or (b) at least 99% identical thereto which encodes SEQ ID NO: 23, and wherein the hSGSH coding sequence is operably linked to regulatory control sequences which direct expression of the hSGSH in a cell. In certain embodiments, the rAAV comprises expression cassette comprising a mature hSGSH coding sequence 99% identical to SEQ ID NO: 16 which is SEQ ID NO: 22 and which encodes SEQ ID NO: 23 (hSGSH.A482Y.E488V). In certain embodiments, the mature hSGSH coding sequence is SEQ ID NO: 16. In certain embodiments, the mature hSGSH coding sequence which is at least about 85% identical to SEQ ID NO: 16, which encodes SEQ ID NO: 23.

In certain embodiments, the regulatory sequences comprise a CNS-specific promoter, e.g., human Synaspin promoter (hSyn), a constitutive promoter, e.g., CB7, CBh or a regulatable promoter. In certain embodiments, the regulatory elements comprise one or more of a Kozak sequence, a TATA signal, an intron, an enhancer, and a polyadenylation sequence.

In certain embodiments, the vector genome comprises nucleic acid sequence of SEQ ID NO: 3 (CB7.CI.BIP.hSGSHcov1-A482Y_E488V.vIGF2.rBG) or a sequence at least 95%, at least 96%, at least 97%, at least 98%, at least 99 to at least 100% identical thereto. In certain embodiments, the vector genome comprises nucleic acid sequence of SEQ ID NO: 7 (CB7.CI.BIP.hSGSHcov1-A482Y_E488V.vIGF2.WPRE.rBG) or a sequence at least 95%, at least 96%, at least 97%, at least 98%, at least 99 to at least 100% identical thereto. In certain embodiments, the vector genome comprises nucleic acid sequence of SEQ ID NO: 10 (CB7.CI.hSGSHcoV1.rBG) or a sequence at least 95%, at least 96%, at least 97%, at least 98%, at least 99 to at least 100% identical thereto. In certain embodiments, the vector genome comprises nucleic acid sequence of SEQ ID NO: 13 (CB7.CI.hSGSHcoV1-4xmiR183.rBG) or a sequence at least 95%, at least 96%, at least 97%, at least 98%, at least 99 to at least 100% identical thereto. In certain embodiments, the vector genome comprises nucleic acid sequence of SEQ ID NO: 40 (CB7.CI.BIP.hSGSHcov1-A482Y_E488V.vIGF2.WPRE.4xmiR182.rBG) or a sequence at least 95%, at least 96%, at least 97%, at least 98%, at least 99 to at least 100% identical thereto. In certain embodiments, the vector genome comprises nucleic acid sequence of SEQ ID NO: 1 (CB7.CI.BIP.hSGSHcov1(A482Y-E488V).vIGF2.WPRE.4xmiR183.rBG) or a sequence at least 95%, at least 96%, at least 97%, at least 98%, at least 99 to at least 100% identical thereto. In certain embodiments, the vector genome comprises nucleic acid sequence of SEQ ID NO: 37 (CB7.CI.BIP.hSGSHcov1-A482Y_E488V.vIGF2.WPRE.4xmiR182.4xmiR183.rBG) or a sequence at least 95%, at least 96%, at least 97%, at least 98%, at least 99 to at least 100% identical thereto.

In certain embodiments, the Clade F AAV capsid is selected from an AAVhu68 capsid [See, e.g., US2020/0056159; PCT/US21/55436; SEQ ID NO: 48 and 49 for nucleic acid sequence; SEQ ID NO: 50 for amino acid sequence], an AAVhu95 capsid [See, e.g., U.S. Provisional Application No. 63/251,599, filed Oct. 2, 2021 and PCT/US2022/077315, filed Sep. 30, 2022; SEQ ID NOs: 54 and 55 (hu95 nucleic acid sequence) and SEQ ID NO: 56 (hu95 amino acid sequence), or an AAVhu96 capsid [See, e.g., U.S. Provisional Application No. 63/251,599, filed Oct. 2, 2021 and PCT/US2022/077315, filed Sep. 30, 2022; SEQ ID NOs: 57 and 58 (hu96 nucleic acid sequence) and SEQ ID NO: 59 (hu96 amino acid sequence). In certain embodiments, the AAV capsid is a Clade A capsid, such as AAVrh91 capsid (nucleic acid sequence of SEQ ID NOs: 51 and 52; amino acid sequence of SEQ ID NO: 53). See, PCT/US20/030266, filed Apr. 29, 2020, now published WO2020/223231, which is incorporated by reference herein and International Application No. PCT/US21/45945, filed Aug. 13, 2021 which are incorporated herein by reference.

In certain embodiments, the AAV capsid for the compositions and methods described herein is chosen based on the target cell. In certain embodiment, the AAV capsid transduces a CNS cell and/or a PNS cell. In certain embodiments, other AAV capsid may be chosen. the AAV capsid is selected from a cy02 capsid, a rh43 capsid, an AAV8 capsid, a rh01 capsid, an AAV9 capsid, an rh8 capsid, a rh10 capsid, a bb01 capsid, a hu37 capsid, a rh02 capsid, a rh20 capsid, a rh39 capsid, a rh64 capsid, an AAV6 capsid, an AAV1 capsid, a hu44 capsid, a hu48 capsid, a cy05 capsid a hu11 capsid, a hu32 capsid, a pi2 capsid, or a variation thereof. In certain embodiments, the AAV capsid is a Clade F capsid, such as AAV9 capsid, AAVhu68 capsid, hu31 capsid, hu32 capsid, or a variation thereof. See, e.g., WO 2005/033321 published Apr. 14, 2015, WO 2018/160582, and US 2015/0079038, each of which is incorporated herein by reference in its entirety. In certain embodiments, the AAV capsid is a non-clade F capsid, for example a Clade A, B, C, D, or E capsid. In certain embodiment, the non-Clade F capsid is an AAV1 or a variation thereof. In certain embodiment, the AAV capsid transduces a target cell other than the nervous system cells. In certain embodiments, the AAV capsid is a Clade A capsid (e.g., AAV1, AAV6, AAVrh91), a Clade B capsid (e.g., AAV 2), a Clade C capsid (e.g., hu53), a Clade D capsid (e.g., AAV7), or a Clade E capsid (e.g., rh10).

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences. The term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

In certain embodiments, the AAV capsid is a AAVhu68 capsid, or an AAVrh91 capsid. In certain embodiments, the AAVhu68 capsid comprises amino acid sequence of SEQ ID NO: 50. In certain embodiments, the AAVhu68 capsid comprises: (i) AAVhu68 vp1 proteins, AAVhu68 vp2 proteins, and AAVhu68 vp3 proteins produced from a nucleic acid sequence encoding SEQ ID NO: 50; or (ii) heterogenous populations of AAVhu68 vp1, AAVhu68 vp2 and AAVhu68 vp3 proteins, wherein the subpopulations of the AAVhu68 vp1, AAVhu68 vp2 and AAV hu68 vp3 proteins comprise at least 50% to 100% deamidated asparagines (N) in asparagine-glycine pairs at each of positions 57, 329, 452, 512, relative to the amino acids in SEQ ID NO: 50, wherein the deamidated asparagines are deamidated to aspartic acid, isoaspartic acid, an interconverting aspartic acid/isoaspartic acid pair, or combinations thereof, as determined using mass spectrometry. In certain embodiments, the nucleic acid sequence encoding AAVhu68 vp1 protein is SEQ ID NO: 48, or a sequence at least 80% to at least 99% identical to SEQ ID NO: 48 which encodes the amino acid sequence of SEQ ID NO: 50; optionally wherein the nucleic acid sequence is at least 80% to 97% identical to SEQ ID NO: 48. In certain embodiments, the nucleic acid sequence encoding AAVhu68 vp1 protein is SEQ ID NO: 49, or a sequence at least 80% to at least 99% identical to SEQ ID NO: 49 which encodes the amino acid sequence of SEQ ID NO: 50; optionally wherein the nucleic acid sequence is at least 80% to 97% identical to SEQ ID NO: 49.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart.

Additionally, provided herein, is an rAAV production system useful for producing a rAAV as described herein. The production system comprises a cell culture comprising (a) a nucleic acid sequence encoding an AAV capsid protein; (b) the vector genome; and (c) sufficient AAV rep functions and helper functions to permit packaging of the vector genome into the AAV capsid. In certain embodiments, the vector genome is SEQ ID NOs: 3, 7, 10, 13, 40, 1, or 37. In certain embodiments, the cell culture is a human embryonic kidney 293 cell culture. In certain embodiments, the AAV rep is from a different AAV. In certain embodiments, wherein the AAV rep is from AAV2. In certain embodiments, the AAV rep coding sequence and cap genes are on the same nucleic acid molecule, wherein there is optionally a spacer between the rep sequence and cap gene.

For use in producing an AAV viral vector (e.g., a recombinant (r) AAV), the vector genomes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

In certain embodiments, a nucleic acid (e.g., a plasmid) useful in rAAV production comprises a vector genome comprising a 5′ ITR, the CB7 promoter sequence, chicken beta actin intron, a Kozak sequence, the BIP.hSGSH.vIGF2 coding sequence, an rBG polyA sequence, and a 3′ ITR. In certain embodiments, a nucleic acid (e.g., a plasmid) useful in rAAV production comprises a vector genome comprising a 5′ ITR, the CB7 promoter sequence, chicken beta actin intron, a Kozak sequence, the BIP.hSGSH.A482Y.E488V.vIGF2 coding sequence, an rBG polyA sequence, and a 3′ ITR. In certain embodiments, a nucleic acid (e.g., a plasmid) useful in rAAV production comprises a vector genome comprising a 5′ ITR, the CB7 promoter sequence, chicken beta actin intron, a Kozak sequence, the BIP.hSGSH.A482Y.E488V.vIGF2 coding sequence, at least 4 tandem repeats miR182, an rBG polyA sequence, and a 3′ ITR. In certain embodiments, a nucleic acid (e.g., a plasmid) useful in rAAV production comprises a vector genome comprising a 5′ ITR, the CB7 promoter sequence, chicken beta actin intron, a Kozak sequence, the BIP.hSGSH.A482Y.E488V.vIGF2 coding sequence, at least 4 tandem repeats miR183 an rBG polyA sequence, and a 3′ ITR. In certain embodiments, a nucleic acid (e.g., a plasmid) useful in rAAV production comprises a vector genome comprising a 5′ ITR, the CB7 promoter sequence, chicken beta actin intron, a Kozak sequence, the BIP.hSGSH.A482Y.E488V.vIGF2 coding sequence, at least 4 tandem repeats miR182, at least four tandem repeats miR183, an rBG polyA sequence, and a 3′ ITR.

Methods for generating and isolating AAVs suitable for use as vectors are known in the art. See generally, e.g., Grieger & Samulski, 2005, Adeno-associated virus as a gene therapy vector: Vector development, production and clinical applications, Adv. Biochem. Engin/Biotechnol. 99: 119-145; Buning et al., 2008, Recent developments in adeno-associated virus vector technology, J. Gene Med. 10:717-733; and the references cited below, each of which is incorporated herein by reference in its entirety. As used herein, a gene therapy vector refers to a rAAV as described herein, which is suitable for use in treating a patient. For packaging a gene into virions, the ITRs are the only AAV components required in cis in the same construct as the nucleic acid molecule containing the gene. The cap and rep genes can be supplied in trans.

In certain embodiments, the manufacturing process for rAAV involves method as described in U.S. Provisional Patent Application No. 63/371,597, filed Aug. 16, 2022, and U.S. Provisional Patent Application No. 63/371,592, filed Aug. 16, 2022, which are incorporated herein by reference in its entirety.

In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV having a capsid selected from AAVhu68, AAVrh91, AAVhu95 or AAVhu96 is provided. Such a cell culture contains a nucleic acid which expresses the AAVhu68 capsid protein in the host cell (e.g., SEQ ID NO: 48 or SEQ ID NO: 49; a nucleic acid molecule suitable for packaging into the AAVhu68 capsid, e.g., a vector genome which contains AAV ITRs and a non-AAV nucleic acid sequence encoding a gene operably linked to regulatory sequences which direct expression of the gene in a host cell; and sufficient AAV rep functions and adenovirus helper functions to permit packaging of the vector genome into the recombinant AAVhu68, or AAVrh91 capsid (e.g., SEQ ID NO: 51 or SEQ ID NO: 52), AAVhu95 capsid (e.g., SEQ ID NO: 54 or SEQ ID NO: 55), AAVhu96 capsid (e.g., SEQ ID NO: 57 or SEQ ID NO: 58). In one embodiment, the cell culture is composed of mammalian cells (e.g., human embryonic kidney 293 cells, among others) or insect cells (e.g., Spodoptera frugiperda (Sf9) cells). In certain embodiments, baculovirus provides the helper functions necessary for packaging the vector genome into the recombinant AAVhu68, AAVrh91, AAVhu95 or AAVhu96 capsid.

Optionally the rep functions are provided by an AAV other than AAV2, selected to complement the source of the ITRs.

In one embodiment, cells are manufactured in a suitable cell culture (e.g., HEK 293 or 59) or suspension. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV vector genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, Adenovirus-adeno-associated virus hybrid for large-scale recombinant adeno-associated virus production, Human Gene Therapy 20:922-929, the contents of each of which is incorporated herein by reference in its entirety. Methods of making and using these and other AAV production systems are also described in the following US patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject further processing including concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. An affinity chromatography purification followed anion exchange resin chromatography are used to purify the vector drug product and to remove empty capsids. These methods are described in more detail in WO2017/160360, filed Dec. 9, 2016, entitled “Scalable Purification Method for AAV9”, which is incorporated by reference. Purification methods for AAV8, WO2017/100676, filed Dec. 9, 2016, and rh10, WO2017/100704, filed Dec. 9, 2016, entitled “Scalable Purification Method for AAVrh10”, also filed Dec. 11, 2015, and for AAV1, WO2017/100674, filed Dec. 9, 2016 for “Scalable Purification Method for AAV1”, filed Dec. 11, 2015, are all incorporated by reference herein. Other suitable methods may be selected.

To calculate empty and full particle content, VP3 band volumes for a selected sample (e.g., in examples herein an iodixanol gradient-purified preparation where # of genome copies (GC)=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

In brief, the method for separating rAAVhu68 (or AAVrh91, AAVhu95 or AAVhu96) particles having packaged genomic sequences from genome-deficient AAVhu68 (or AAVrh91 or AAVhu95 or AAVhu96) intermediates involves subjecting a suspension comprising recombinant AAVhu68 (or AAVrh91) viral particles and AAVhu68 (or AAVrh91 or AVhu95 or AAVhu96) capsid intermediates to fast performance liquid chromatography, wherein the AAVhu68 (or AAVrh91 or AAVhu95 or AAVhu96) viral particles and AAVhu68 intermediates are bound to a strong anion exchange resin equilibrated at a pH of about 10.2 (or about 9.8 for AAVrh91), and subjected to a salt gradient while monitoring eluate for ultraviolet absorbance at about 260 nanometers (nm) and about 280 nm. Although less optimal for rAAVhu68 and AAVrh91, the pH may be in the range of about 10 to 10.4. In this method, the AAV full capsids are collected from a fraction which is eluted when the ratio of A260/A280 reaches an inflection point. In one example, for the Affinity Chromatography step, the diafiltered product may be applied to an affinity resin (Life Technologies) that efficiently captures the AAV serotype. Under these ionic conditions, a significant percentage of residual cellular DNA and proteins flow through the column, while AAV particles are efficiently captured.

The rAAV.hSGSH (e.g., rAAV.BIP.hSGSH or rAAV.BIP.hSGSH.vIGF2, or rAAV.BIP.hSGSH.A482Y.E488V or rAAV.BIP.hSGSH.A482Y.E488V.vIGF2) is suspended in a suitable physiologically compatible composition (e.g., a buffered saline). This composition may be frozen for storage, later thawed and optionally diluted with a suitable diluent. Alternatively, the vector may be prepared as a composition which is suitable for delivery to a patient without proceeding through the freezing and thawing steps.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 June; 78(10): 6381-6388, which identifies Clades A, B, C, D, E and F, and provides nucleic acid sequences of novel AAV, GenBank Accession Numbers AY530553 to AY530629. See, also, WO 2005/033321.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, the terms “rAAV” and “artificial AAV” used interchangeably, mean, without limitation, a AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

The term “heterogeneous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

### Pharmaceutical Composition

In one aspect, provided herein is a pharmaceutical composition comprising a vector as described herein in a formulation buffer. In one embodiment, provided is a pharmaceutical composition comprising a rAAV as described herein in a formulation buffer. In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least about 1×10¹¹ GC/mL.

Provided herein also is a composition comprising an rAAV or a vector as described herein and an aqueous suspension media. In certain embodiments, the suspension is formulated for intravenous delivery, intrathecal administration, or intracerebroventricular administration. In one aspect, the compositions contain at least one rAAV stock and an optional carrier, excipient and/or preservative. As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits x 100 give the approximate molecular mass of the polyoxypropylene core, and the last digit x 10 gives the percentage polyoxyethylene content. In one embodiment Poloxamer 188 is selected. In one embodiment, the surfactant may be present in an amount up to about 0.0005% to about 0.001% (based on weight ratio, w/w %) of the suspension. In another embodiment, the surfactant may be present in an amount up to about 0.0005% to about 0.001% (based on volume ratio, v/v %) of the suspension. In yet another embodiment, the surfactant may be present in an amount up to about 0.0005% to about 0.001% of the suspension, wherein n % indicates n gram per 100 mL of the suspension.

In another embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrol® F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits x 100 give the approximate molecular mass of the polyoxypropylene core, and the last digit x 10 gives the percentage polyoxyethylene content. In one embodiment Poloxamer 188 is selected. The surfactant may be present in an amount up to about 0.0005% to about 0.001% of the suspension.

In certain embodiments, the composition containing the rAAV.hSGSH is delivered at a pH in the range of 6.8 to 8, or 7.2 to 7.8, or 7.5 to 8. For intrathecal delivery, a pH above 7.5 may be desired, e.g., 7.5 to 8, or 7.8.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. In one embodiment, the buffer is PBS. In another embodiment, the buffer is an artificial cerebrospinal fluid (aCSF), e.g., Eliott's formulation buffer; or Harvard apparatus perfusion fluid (an artificial CSF with final Ion Concentrations (in mM): Na 150; K 3.0; Ca 1.4; Mg 0.8; P 1.0; Cl 155). The aqueous solution may further contain Kolliphor® P188, a poloxamer which is commercially available from BASF which was formerly sold under the trade name Lutrol® F68. The aqueous solution may have a pH of 7.2.

In another embodiment, the formulation may contain a buffered saline aqueous solution comprising 1 mM Sodium Phosphate (Na3PO4), 150 mM sodium chloride (NaCl), 3 mM potassium chloride (KCl), 1.4 mM calcium chloride (CaCl2)), 0.8 mM magnesium chloride (MgCl2), and 0.001% poloxamer (e.g., Kolliphor®) 188, pH 7.2. See, e.g., harvardapparatus.com/harvard-apparatus-perfusion-fluid.html. In certain embodiments, Harvard's buffer is preferred due to better pH stability observed with Harvard's buffer.

In certain embodiments, the formulation buffer is artificial CSF with Pluronic F68. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In certain embodiments, the ommaya reservoir is used for delivery. In one example, the composition is formulated for intrathecal delivery. In one example, the composition is formulated for intravenous (iv) delivery.

In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In one embodiment, the pharmaceutical composition comprising a rAAV as described herein is administrable at a dose of about 1×10⁹ GC per gram of brain mass to about 1×10¹³ GC per gram of brain mass.

It should be understood that the compositions in the pharmaceutical composition described herein are intended to be applied to other compositions, regiments, aspects, embodiments and methods described across the Specification.

### Uses

In one aspect, a method is provided herein is a method of treating a human subject diagnosed with MPS IIIA. Currently, when there is a clinical suspicion of MPS III, the first step is the request of a quantitative test to detect the presence of GAGs in urine through spectrophotometric methods using dimethylmethylene blue (DMB). The DMB test is based on the union of GAGs to the dimethylmethylene blue and the quantification of the GAG-DMB complex with a spectrophotometer. The sensitivity of this test is 100%, with a specificity of 75-100%. A negative result when detecting GAGs in urine does not rule out the existence of MPS III due to the fact that in some patients with attenuated forms of the disease, the levels of GAGs excretion with healthy controls can overlap and the increased excretion of heparan sulfate in the MPS III can be ignored. The current gold standard technique for diagnosis is the determination of enzyme activity in cultured skin fibroblasts, leukocytes, plasma or serum. The specific diagnosis of MPS IIIA is confirmed by showing a decrease or absence of one of the SGSH enzymatic activities involved in the degradation of heparan sulfate in the patient's leukocytes or fibroblasts; the reduction should be less than 10% when compared to the activity in healthy individuals, with normalcy in other sulfatases. Because the disease due to deficiency in multiple sulfatases also shows a reduction in the activity of the heparan N-sulfatase, N-acetylglucosamine 6-sulfatase and other sulfatases, biochemical analysis of at least other sulfatase is required to confirm the diagnosis of MPS III and thus rule out multiple sulfatases deficiency. However, the method of diagnosis is not a limitation of the present invention and other suitable methods may be selected.

The method comprises administering to a subject a suspension of a vector as described herein. In one embodiment, the method comprises administering to a subject a suspension of a rAAV as described herein in a formulation buffer at a dose of about 1×10⁹ GC per gram of brain mass to about 1×10¹⁴ GC per gram of brain mass.

The composition(s) and method(s) provided herein achieve efficacy in treating a subject in need with MPS IIIA. Efficacy of the method in a subject can be shown by assessing (a) an increase in SGSH enzymatic activity; (b) amelioration of a MPS IIIA symptom; (c) improvement of MPS IIIA-related biomarkers, e.g., GAG levels polyamine (e.g., spermine) levels in the cerebrospinal fluid (CSF), serum, urine and/or other biological samples; or (e) facilitation of any treatment(s) for MPS IIIA. In certain embodiments, efficacy may be determined by monitoring cognitive improvement and/or anxiety correction, gait and/or mobility improvement, reduction in tremor frequency and/or severity, reduction in clasping/spasms, improvements in posture, improvements in corneal opacity. Additionally or alternatively, efficacy of the method may be predicted based on an animal model. In another embodiment, a multiparameter grading scale was developed to evaluate disease correction and response to the MPSIIIA vector therapy described herein in an animal model. Animals are assigned a score based on an assessment of a combination of tremor, posture, fur quality, clasping, corneal clouding, and gait/mobility. In certain embodiments, any combination of one or more of these factors may be used to demonstrate efficacy, alone, or in combination with other factors. See, e.g., Burkholder et al. Curr Protoc Mouse Biol. June 2012, 2:145-65; Tumpey et al. J Virol. May 1998, 3705-10; and Guyenet et al. J Vis Exp, May 2010, 39; 1787). Cognitive improvement and anxiety correction of treated animals is evaluated by assessing movement in an open field (i.e., beam break measurement as described, e.g., in Tatem et al. J Vis Exp, 2014, (91):51785) and the elevated plus maze assay (as described, e.g., in Walf and Frye, Nat Protoc, 2007, 2(2): 322-328.

As used herein, “facilitation of any treatment(s) for MPS IIIA” or any grammatical variant thereof, refers to a decreased dosage or a lower frequency of a treatment of MPS IIIA in a subject other than the composition(s) or method(s) which is/are firstly disclosed in the invention, compared to that of a standard treatment without administration of the described composition(s) and use of the described method(s). Examples of suitable treatment facilitated by the composition(s) or method(s) described herein might include, but not limited to, medications used to relieve symptoms (such as seizures and sleep disturbances) and improve quality of life; hematopoietic stem cell transplantation, such as bone marrow transplantation or umbilical cord blood transplantation; enzyme replacement therapies (ERT) via intravenous administration or intracerebroventricular infusion; and any combination thereof. In one embodiment, the described method results in the subject demonstrating an improvement of biomarkers related to MPS IIIA.

An “increase in SGSH enzymatic activity” is used interchangeably with the term “increase in desired SGSH function”, and refers to a SGSH activity at least about 5%, 10%, 15%, 20%, about 25%, about 30%, about 35%, about 40%, about 45%, about 50%, about 55%, about 60%, about 65%, about 70%, about 75%, about 80%, about 85%, about 90%, about 95%, or about 100% of the SGSH enzyme range for a healthy patient. The SGSH enzymatic activity might be measured by an assay as described herein. In one embodiment, the SGSH enzymatic activity might be measured in the serum, plasma, blood, urine, CSF, or another biological sample. In one embodiment, administration of the composition as described herein, or use of the method as described herein, result in an increase in SGSH enzymatic activity in serum, plasma, saliva, urine or other biological samples. Alternatively, CSF GAG levels and other CSF biomarkers such as spermine levels may be measured to determine therapeutic effect. See. e.g., WO 2017/136533.

Neurocognition can be determined by conventional methods, See. e.g., WO 2017/136500 A1. Prevention of neurocognitive decline refers to a slowdown of a neurocognitive decline of the subject administered with the composition described herein or received the method described herein by at least about 5%, at least about 20%, at least about 20%, about 25%, about 30%, about 35%, about 40%, about 45%, about 50%, about 55%, about 60%, about 65%, about 70%, about 75%, about 80%, about 85%, about 90%, about 95%, or about 100% compared to that of a MPS IIIA patient.

As used herein, the terms “biomarker” or “MPS IIIA-related biomarker” refer to presence, concentration, expression level or activity of a biological or chemical molecular in a biological sample of a subject which correlates to progression or development of MPS IIIA in a positive or negative matter. In one embodiment, the biomarker is GAG levels in the cerebrospinal fluid (CSF), serum, urine, skin fibroblasts, leukocytes, plasma, or any other biological samples. In another embodiment, the biomarker is assessed using clinical chemistry. In yet another embodiment, the biomarker is liver or spleen volumes. In one embodiment, the biomarker is the activity of the heparan N-sulfatase, N-acetylglucosamine 6-sulfatase and other sulfatases. In another embodiment, the biomarker is spermine level in CSF, serum, or another biological sample. In yet another embodiment, the biomarker is lysosomal enzyme activity in serum, CSF, or another biological sample. In one embodiment, the biomarker is assessed via magnetic resonance imaging (MRI) of brain. In another embodiment, the biomarker is a neurocognitive score measured by a neurocognitive developmental test. The phrase “improvement of biomarker” as used herein means a reduction in a biomarker positively correlating to the progression of the disease, or an increase in a biomarker negatively correlating to the progression of the disease, wherein the reduction or increase is at least about 5%, at least about 20%, at least about 20%, about 25%, about 30%, about 35%, about 40%, about 45%, about 50%, about 55%, about 60%, about 65%, about 70%, about 75%, about 80%, about 85%, about 90%, about 95%, or about 100% compared to that before administration of the composition as described herein or use of the method as described herein.

In one embodiment, the method further comprises detecting or monitoring biomarkers related to MPS IIIA in the subject prior to initiation of therapy with therapy provided herein. In certain embodiments, the method comprises detection of biomarker which is a polyamine (such as spermine) in a sample from a subject (see WO/2017/136533, which is incorporated herein by reference). In certain embodiments, spermine concentration levels in a patient sample are detected to monitor the effectiveness of a treatment for MPSIII using the vector as described herein.

Currently, patients with MPSIIIA are not considered candidates for bone marrow transplantation (BMT), Substrate Reduction Therapy (SRT) or enzyme replacement therapy (ERT). However, in certain embodiments, a gene therapy patient treated with a vector expressing the SGSH described herein has, at a minimum, sufficient enzyme expression levels that any sub-normal range enzyme levels can be treated with ERT or SRT. Such ERT may be a co-therapy in which the dose of the ERT is monitored and modulated for months or years post-vector dosing. Additionally or alternatively, a SRT may be a co-therapy in which the dose of the SRT is monitored and modulated for months or years post-vector dosing.

As used herein, an enzyme replacement therapy (ERT) is a medical treatment that consists in replacing an enzyme in patients where a particular enzyme is deficient or absent. The enzyme is usually produced as a recombinant protein and administrated to the patient. In one embodiment, the enzyme is a functional SGSH. In another embodiment, the enzyme is a recombinant protein comprising a functional SGSH. Systemic, intrathecal, intracerebroventricular or intracisternal delivery can be accomplished using ERT. As used herein, a Substrate Reduction Therapy (SRT) refers to a therapy using a small molecule drug to partially inhibit the biosynthesis of the compounds, which accumulate in the absence of SGSH. In one embodiment, the SRT is a therapy via genistein. See, e.g., Ritva Tikkanen et al, Less Is More: Substrate Reduction Therapy for Lysosomal Storage Disorders. Int J Mol Sci. 2016 July; 17(7): 1065. Published online 2016 Jul. 4. doi: 10.3390/ijms17071065; Delgadillo V et al, Genistein supplementation in patients affected by Sanfilippo disease. J Inherit Metab Dis. 2011 October; 34(5):1039-44. doi: 10.1007/s10545-011-9342-4. Epub 2011 May 10; and de Ruijter J et al, Genistein in Sanfilippo disease: a randomized controlled crossover trial. Ann Neurol. 2012 January; 71(1):110-20. doi: 10.1002/ana.22643.

Suitable volumes for delivery of these doses and concentrations may be determined by one of skill in the art. For example, volumes of about 1 μL to 150 mL may be selected, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

In one embodiment, the rAAV as described herein is administrable at a dose of about 1×10⁹ GC per gram of brain mass to about 1×10¹⁴ GC per gram of brain mass. In certain embodiments, the rAAV is co-administered systemically at a dose of about 1×10⁹ GC per kg body weight to about 1×10¹³ GC per kg body weight

In one embodiment, the subject is delivered a therapeutically effective amount of the vectors described herein. As used herein, a “therapeutically effective amount” refers to the amount of the composition comprising the nucleic acid sequence encoding hSGSH which delivers and expresses in the target cells an amount of enzyme sufficient to achieve efficacy. In one embodiment, the dosage of the vector is about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹³ GC/g, about 9.5×10¹³ GC/g, or about 1.0×10¹⁴ GC/g brain mass.

The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the minigene. Optionally, dosage regimens similar to those described for therapeutic purposes may be utilized for immunization using the compositions of the invention.

In one embodiment, the method further comprises the subject receives an immunosuppressive co-therapy. Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25−) or CD3-directed antibodies, anti-IL-2 antibodies, ciclosporin, tacrolimus, sirolimus, IFN-β, IFN-γ, an opioid, or TNF-α (tumor necrosis factor-alpha) binding agent.

In certain embodiments, the immunosuppressive therapy may be started 0, 1, 2, 7, or more days prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

In certain embodiment, the method comprises measurement of serum anti-hSGSH antibodies. Suitable assays of measuring anti-hSGSH antibody are available as described herein.

In one embodiment, the rAAV as described herein is administrated once to the subject in need. In another embodiment, the rAAV is administrated more than once to the subject in need.

The above-described recombinant vectors may be delivered to host cells according to published methods. The rAAV, preferably suspended in a physiologically compatible carrier, may be administered to a human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. However, other pHs within the broadest ranges and these subranges may be selected for other route of delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna. In certain embodiment, a rAAV, vector, or composition as described herein is administrated to a subject in need via the intrathecal administration. In certain embodiments, the intrathecal administration is performed as described in US Patent Publication No. 2018-0339065 A1, published Nov. 29, 2019, which is incorporated herein by reference in its entirety.

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

In certain embodiments, treatment of the composition described herein has minimal to mild asymptomatic degeneration of DRG sensory neurons in animals and/or in human patients, well-tolerated with respect to sensory nerve toxicity and subclinical sensory neuron lesions.

It should be understood that the compositions in the method described herein are intended to be applied to other compositions, regiments, aspects, embodiments and methods described across the Specification.

### Kit

In certain embodiments, a kit is provided which includes a concentrated vector suspended in a formulation (optionally frozen), optional dilution buffer, and devices and components required for intrathecal, intracerebroventricular or intracisternal administration. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in kit described herein are intended to be applied to other compositions, regiments, aspects, embodiments and methods described across the Specification.

Apparatus and Method for Delivery of a Pharmaceutical Composition into Cerebrospinal Fluid

In one aspect, the vectors, rAAV or compositions thereof provided herein may be administered intrathecally via the method and/or the device provided in this section and described in WO 2017/136500 and WO 2018/160582, which are incorporated by reference herein. Alternatively, other devices and methods may be selected. In certain embodiments, the method comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis. In certain embodiments, vectors and/or compositions thereof as described herein are administered via computed tomography- (CT-) guided sub-occipital injection into the cisterna magna (intra-cisterna magna [ICM]). In certain embodiments, the apparatus is described in US Patent Publication No. 2018-0339065 A1, published Nov. 29, 2019, which is incorporated herein by reference in its entirety. In certain embodiments, the vectors, rAAV or compositions thereof provided herein may be administered using Ommaya Reservoir.

It should be understood that the compositions in the device described herein are intended to be applied to other compositions, regiments, aspects, embodiments and methods described across the Specification.

As used herein, the term “administration” or any grammatical variations thereof refers to delivery of composition described herein to a subject.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be included and described using “consisting of” or “consisting essentially of” language. As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an” refers to one or more. As such, the terms “a” (or “an”), “one or more,” and “at least one” are used interchangeably herein.

As used herein, the term “about” or “˜” refers to a variant of 10% from the reference integer and values therebetween, unless otherwise specified. For example, “about” 500 μM includes ±50 (i.e., 450-550, which includes the integers therebetween). For other values, particularly when reference is to a percentage (e.g., 90% of taste), the term “about” is inclusive of all values within the range including both the integer and fractions.

As described above, the term “about” when used to modify a numerical value means a variation of 10%, (±10%, e.g., ±1, ±2, ±3, ±4, ±5, ±6, ±7, ±8, ±9, ±10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are provided to illustrate certain aspects of the claimed invention. The invention is not limited to these examples.

This is a method to treat mucopolysaccharidosis type IIIA (MPSIIIA, or Sanfilippo A syndrome) using a recombinant adeno-associated virus serotype hu68 (or other pantropic/neurotropic serotype) with a ubiquitous promoter expressing an engineered version of human N-sulfoglucosamine sulfohydrolase (hSGSHco) with a polyA sequence. Several engineered sequences encoding versions of the hSGSH are covered: reference amino acid sequence (N-sulphoglucosamine sulphohydrolase isoform 1 precursor [Homo sapiens] NCBI Reference Sequence: NP_000190.1); another frequent missense single nucleotide polymorphism rs7503034 encoding for NP_000190.1:p.Arg456Pro; fusion protein vIGF2-hSGSHco; fusion protein vIGF2-hSGSHco(R456P). More details about the sequences are encompassed herein throughout application.

The vector is administered via intracerebroventricular (ICV) or intrathecal (IT), delivery. IT delivery covers both the lumbar route and the suboccipital cisterna magna. The rationale is to drive high hSGSH expression in the CNS to correct the neurologic manifestations of the MPSIIIA disease.

In addition, thorough coding sequence engineering optimization and optimization of the protein (testing alternative SNPs, engineering the targeting peptide etc.) provides an efficient gene therapy strategy allowing to lower the minimal efficacy dose compared to traditional approaches using WT protein or first-generation approaches using different coding sequence engineering process.

### Example 1. Comparison of Engineered SGSH in WT Mice and Engineered SGSH Constructs In Vitro

In this study we used, we compared engineered hSGSH coding sequence for expression (i.e., quantification) and activity. The hSGSH activity was assayed in obtained serum samples on day 7 and day 28.

Various SGSH assays examining for activity and quantification of SGSH are available. One SGSH assay is a fluorescence-based SGSH activity assay, in which a pure, commercial-grade SGSH, conditioned media, and a mouse tissue homogenate are used. Another SGSH assay is HPLC-based SGSH activity assay, which requires further optimization. Yet another SGSH assay is a Mass spec-based SGSH activity assay, which also requires further optimization. Additionally, a Mass spec-based Signal Peptide SGSH protein quantitation assay can be used.

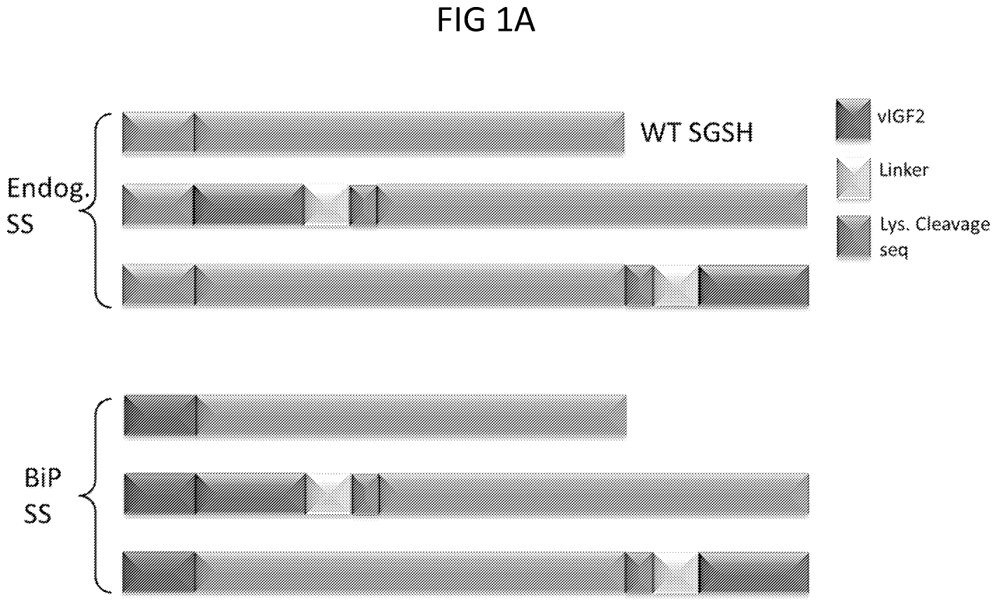

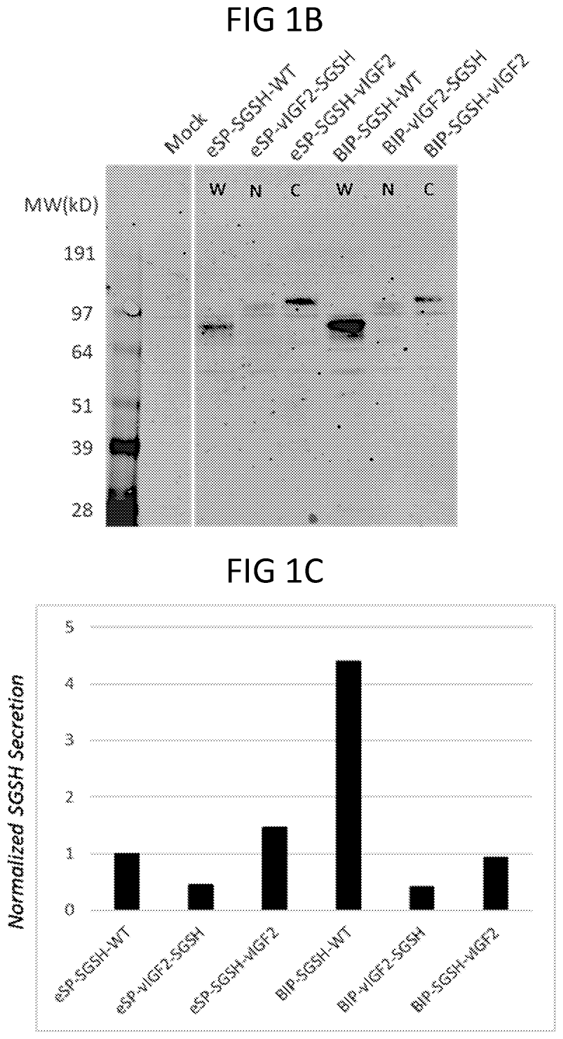

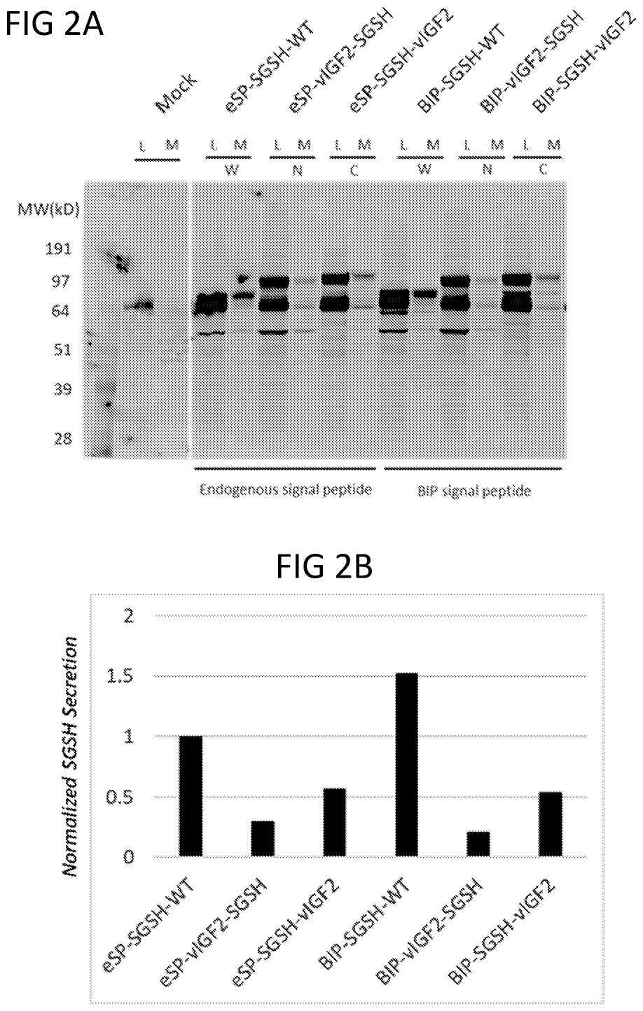

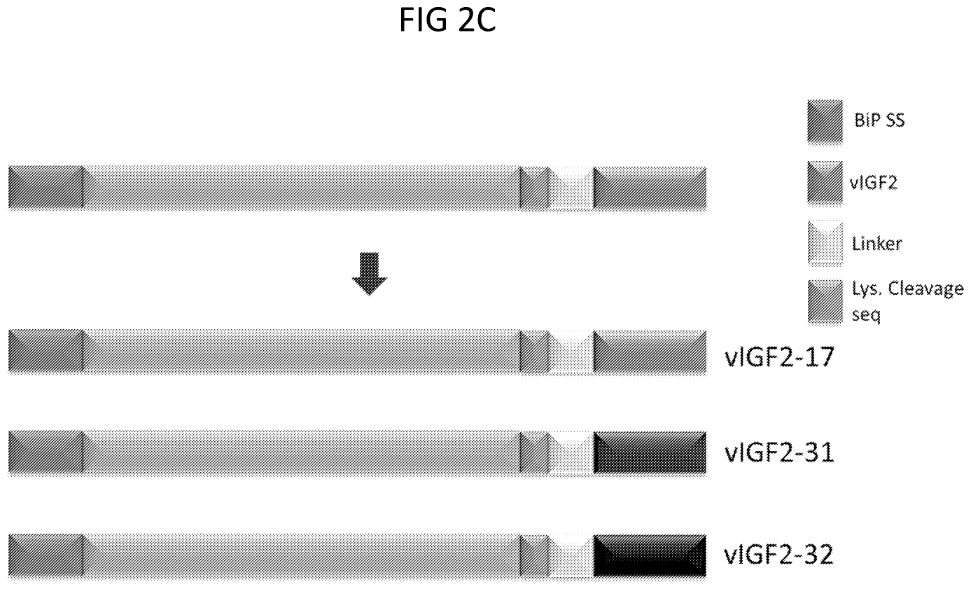

First, we examined the construct in vitro. For this study, we tested various hSGSH constructs for expression in HEK293 cells (FIG. 1A). FIG. 1A shows various designed hSGSH constructs, comprising wild-type (WT) mature SGSH coding sequence, and further comprising either endogenous signal sequence or binding immunoglobulin protein (BIP) signal sequence, linker, vIGF2 peptide (i.e., peptide that binds CI-MPR), and/or lysosomal cleavage sequence. HEK293 cells were transfected with plasmid comprising nucleic acid sequence encoding constructs as described in FIG. 1A. The collected media from HEK293 cells was then analyzed for expression and secretion of SGSH constructs at 3-days and 4-days post-transfection. FIG. 1B shows expression levels of SGSH secreted into HEK293 cell media at 3-days post transfection, as analyzed using western blot. FIG. 1C shows quantified SGSH expression levels (from western blot analysis; FIG. 1B), plotted as normalized SGSH secretion levels. FIG. 2A shows expression levels of SGSH secreted into HEK293 cell media at 4-days post transfection, as analyzed using western blot. FIG. 2B shows quantified SGSH expression levels (from western blot analysis; FIG. 2A), plotted as normalized SGSH secretion levels. Furthermore, we engineered the mature SGSH coding sequence (FIG. 2C) for use in BiP signal sequence- and vIGF2 peptide-comprising constructs.

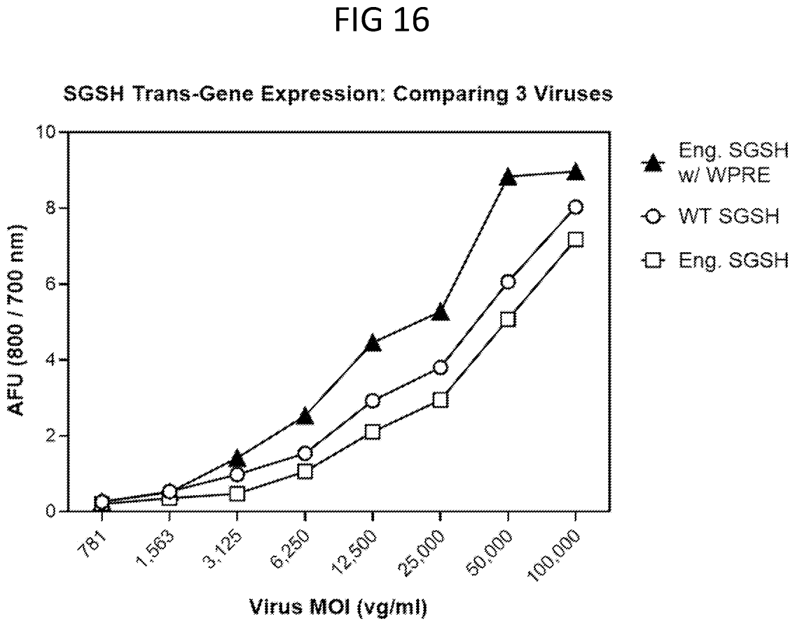

We further examined in vitro expression of AAV plasmids comprising WT and engineered SGSH transgene in HEK293F cells. FIG. 16A shows expression levels of SGSH secreted into HEK293 cell media following transfection with AAV plasmids comprising engineered SGSH construct, engineered SGSH construct with WPRE element, and WT SGSH construct, as analyzed using western blot. FIG. 16B shows AAV quantified SGSH expression (normalized to WT SGSH) following transfection of HEK293F cells with AAV plasmids. These results show that inclusion of WPRE element increased engineered SGSH expression by approximately 40% (n=1). As reference, FIGS. 16C and 16D show the typical expression observed with mammalian expression vector. FIG. 16C shows SGSH expression levels following transfection with plasmid comprising SGSH construct with and without vIGF2 peptide, as examined with western blot analysis. FIG. 16D shows SGSH expression levels as quantified from western blot analysis, and plotted normalized to WT SGSH expression levels. These results show increase in expression levels of SGSH in SGSH constructs comprising vIGF2.

Next, we examined engineered constructs in vivo in WT mice. For this study, mice (age 1-2 months) were administered with rAAV having an AAVhu68 capsid and comprising vector genome which comprises various hSGSH coding sequences (i.e., WT and engineered; also referenced to as AAVhu68.hSGSH), the expression of which is driven by CB7 promoter.

The injection was performed on Day 0 of the study. Serum samples were collected on Day 7 and Day 28 of the study. Necropsy was performed on Day 28, during which brain and liver tissues were collected. The serum samples from day 7 post injection were collected and stored at −80° C. On day 28, necropsy was performed, and samples were collected and stored at −80° C. More specifically, on day 28 post injection serum samples were collected instead of plasma samples. The stored samples included brain (right half) tissue and liver tissues, which were analyzed for hSGSH enzyme activity and expression (i.e., western blot). Other half of liver and brain (left) tissues were fixed in formalin and transferred to pathology core for hSGSH IHC. Table immediately below summarizes the study layout.

| | | Dose | Number of |
|---|---|---|---|
| Group | Treatment | (GC/mouse) | Animals |
| 1 | CO Version 1 | 1 × 10¹⁰ GC | 4 |
| | | 1 × 10¹¹ GC | 4 |
| 2 | CO Version 2 | 1 × 10¹⁰ GC | 4 |
| | with R456P | 1 × 10¹¹ GC | 4 |
| 3 | CO Version 2 | 1 × 10¹⁰ GC | 4 |
| | | 1 × 10¹¹ GC | 4 |
| 4 | CO Version 3 | 1 × 10¹⁰ GC | 4 |
| | | 1 × 10¹¹ GC | 4 |
| 5 | PBS | 1 × 10¹⁰ GC | 4 |
| | | 1 × 10¹¹ GC | 4 |

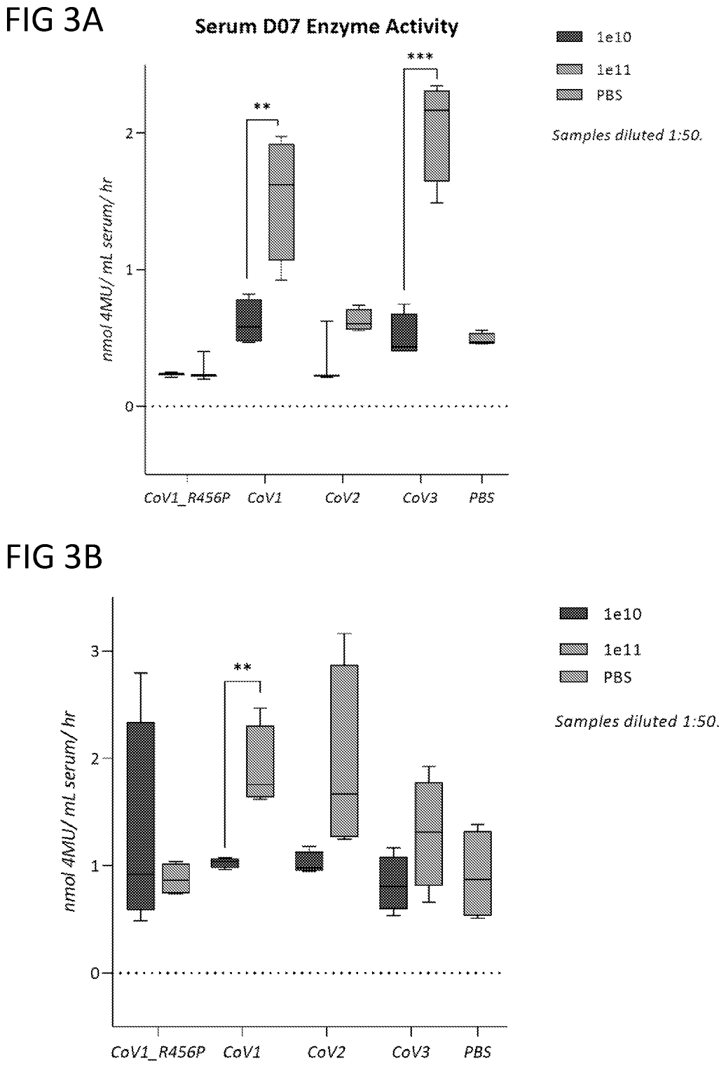

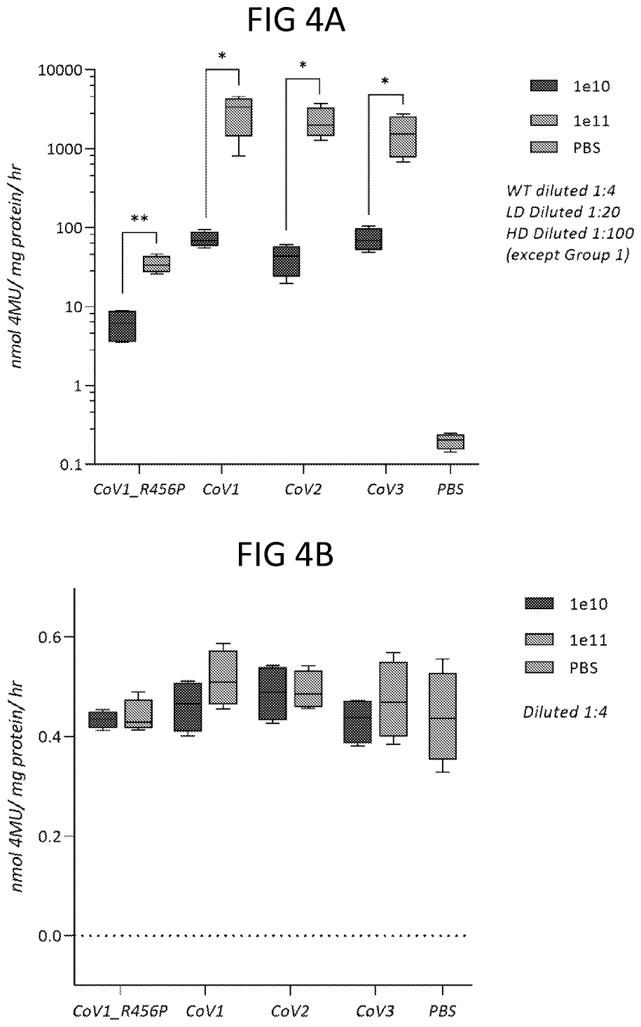

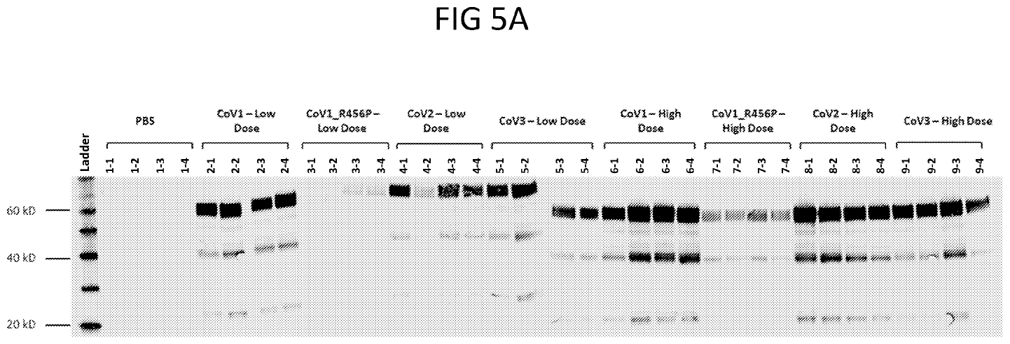

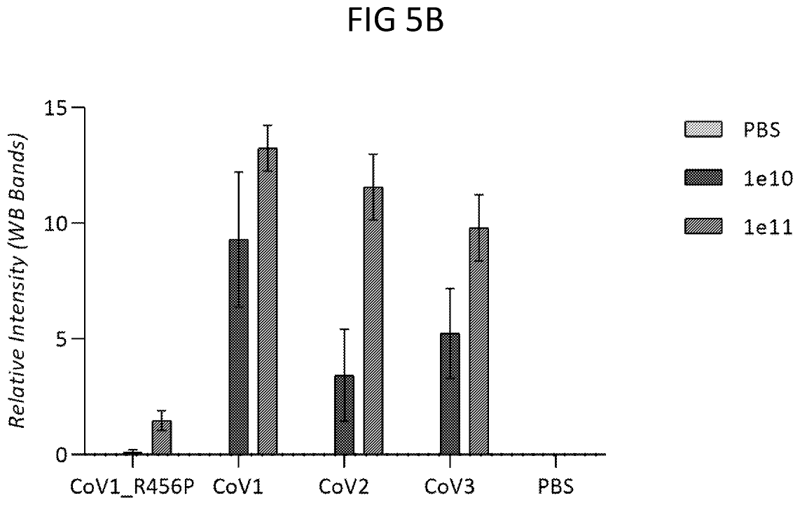

SGSH enzyme activity was analyzed in serum on Day 7 and Day 28 of the study using fluorescent activity assay. The collected serum samples were diluted 1:50. FIG. 3A shows SGSH enzyme activity from serum samples collected from mice administered with AAVhu68.hSGSH (1×10¹⁰ GC or 1×10¹¹ GC) on Day 7 of the study. FIG. 3B shows SGSH enzyme activity from serum samples collected from mice administered with AAVhu68.hSGSH (1×10¹¹ GC or 1×10¹¹ GC) on Day 28 of the study. These results show that about two-fold increase was observed between SGSH concentrations in serum for treated vs. PBS-treated (control groups). Furthermore. We examined SGSH enzyme activity from brain and liver tissue collected from mice at necropsy. FIG. 4A shows SGSH enzyme activity from homogenate liver tissue samples collected from mice administered with 1×10¹⁰ GC and 1×10¹¹ GC. FIG. 4B shows SGSH enzyme activity from homogenate brain tissue samples collected from mice administered with 1×10¹⁰ GC and 1×10¹¹ GC. These results confirm liver transduction, as significant SGSH activity was observed in liver tissue homogenate samples. The SGSH activity in brain tissue homogenate was not statistically greater than WT. We further examined SGSH expression levels in liver tissue homogenate samples by western blot. FIG. 5A shows expression levels of SGSH as analyzed from liver tissue homogenate samples from mice administered with AAVhu68.hSGSH at doses of 1×10¹⁰ GC and 1×10¹¹ GC. FIG. 5B shows quantified expression levels of SGSH as analyzed by western blot (shown in FIG. 5A). Western Blot analysis of brain homogenates showed no visible banding at same intensity as liver. Bands revealed at high contrast with no distinguishing features between groups (blots not shown). Immunohistochemical microscopy analysis was performed of liver tissue collected from mice administered with AAVhu68.hSGSH comprising various constructs (CoV1, CoV1-R456P, CoV2, CoV3) at a dose of 1×10¹¹ GC, at a dose of 1×10¹⁰ GC or treated with PBS (data not shown). These results confirm the hSGSH expression in liver tissue as analyzed by western blot.

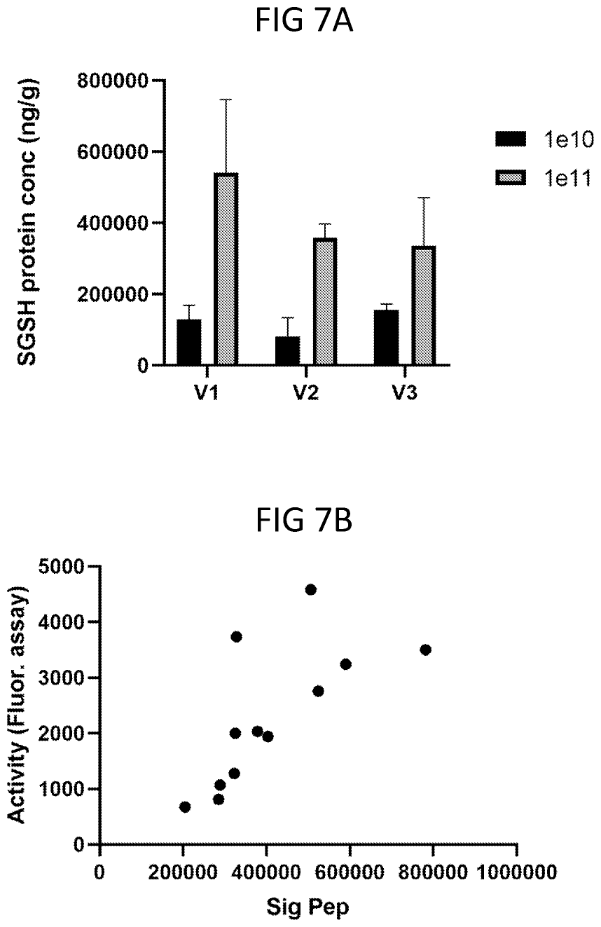

Additionally, we performed signal peptide assay on SGSH in liver tissue samples. FIG. 7A shows results of the SGSH protein concentration, plotted as ng/g, as analyzed from collected liver tissue samples from mice which were administered with AAVhu68.hSGSH at doses of 1×10¹⁰ GC to 1×10¹¹ GC. FIG. 7B shows correlation between SGSH enzyme activity (as measured using fluorescence assay) and Signal Peptide assay performed on collected liver samples from mice administered with high dose of AAVhu68.hSGSH (FIG. 7A; 1×10¹¹ GC).

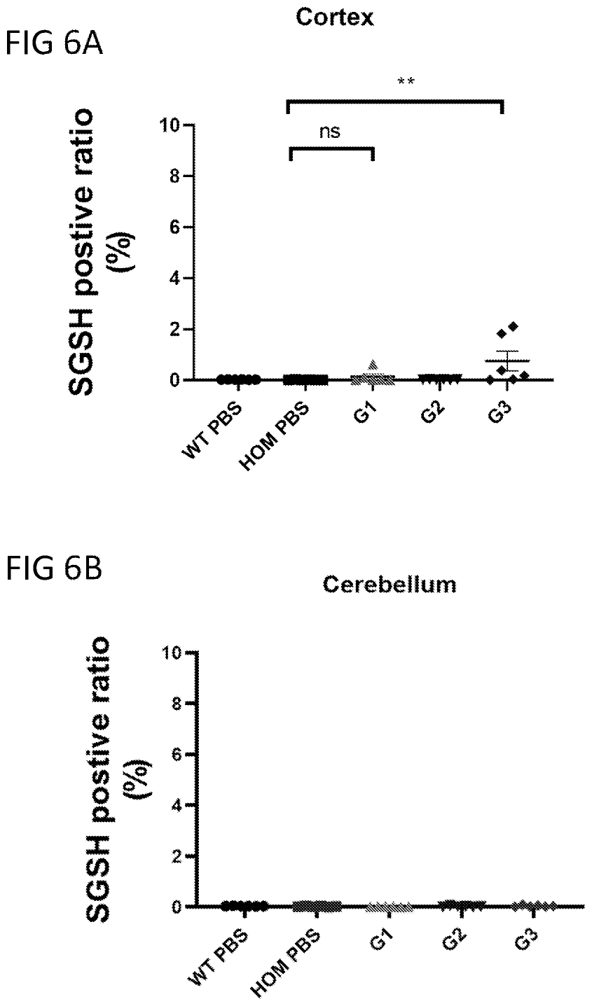

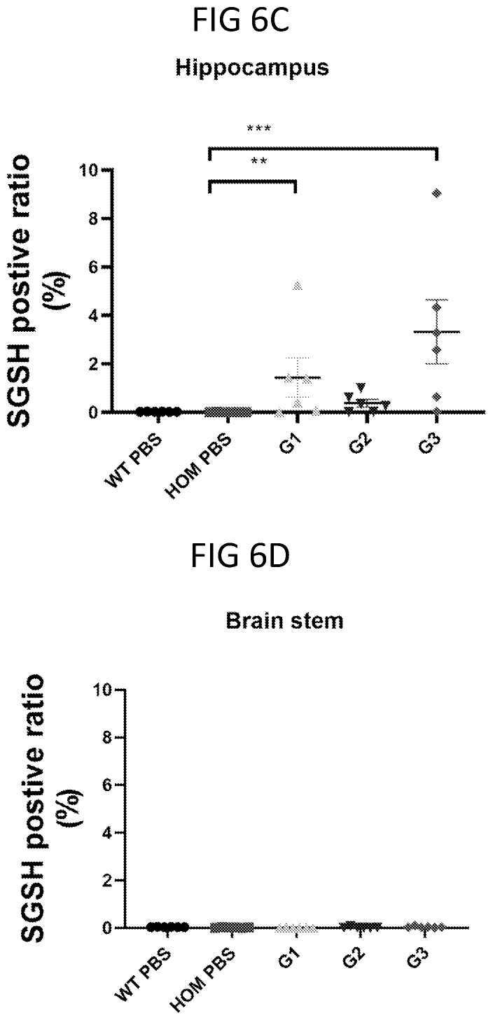

Additionally, we examined the effect of WPRE element in the vector genome. FIG. 6A shows SGSH expression and quantification in cortex following administration of AAVhu68.hSGSH with WPRE. FIG. 6B shows SGSH expression and quantification in cerebellum following administration of AAVhu68.hSGSH with WPRE. FIG. 6C shows SGSH expression and quantification in hippocampus following administration of AAVhu68.hSGSH with WPRE. FIG. 6D shows SGSH expression and quantification in brain stem following administration of AAVhu68.hSGSH with WPRE. These results show that addition of WPRE to the engineered candidate rescues expression to levels similar to the WT construct.

In summary, when the rAAVhu68.hSGSH constructs was administered at high dose (HD; 1×10¹¹ GC) or lose dose (LD; 1×10¹⁰ GC), neither group shows significant transduction of brain. Analysis of liver tissue homogenate samples shows significant transduction in all treatment groups.

Construct “SGSH-CoV1” (or CoV1) showed slightly higher activity in liver than other CohSGSH constructs (e.g., CoV1-R456P, CoV2, CoV3), and showed statistically different levels of activity between high and low doses at both day 7 and day 28 in serum. Furthermore, we confirmed that Arg456Pro is not a benign variant. The Western blot analysis corroborates activity assay data.

Production of rAAV Comprising hSGSH

In the studies herein, a fusion protein comprising an exogenous BIP leader and/or vIGF peptide fused to engineered sequences encoding human SGSH were generated, and comparative studies were performed with the corresponding construct without the exogenous BIP peptide and vIGF2 peptide sequences. The rAAV are generated using triple transfection techniques, utilizing (1) a cis plasmid encoding AAV2 rep proteins and the AAVhu68 VP1 cap gene, (2) a cis plasmid comprising adenovirus helper genes not provided by the packaging cell line which expresses adenovirus Ela, and (3) a trans plasmid containing the vector genome for packaging in the AAV capsid. See, e.g., US 2020/0056159. The trans plasmid is designed to contain either the vector genome comprising hSGSH with exogenous leader peptide (BIP) and hSGSH with the native hSGSH leader peptide with and without vIGF2 peptide. The vector genomes include:
(i) CB7.CI.BIP.hSGSHcov1(A482Y-E488V).vIGF2.WPRE.4xmiR183.rBG (SEQ ID NO: 1);
(ii) CB7.CI.BIP.hSGSHcov1-A482Y_E488V.vIGF2.rBG (SEQ ID NO: 3);
(iii) CB7.CI.BIP.hSGSHcov1-A482Y_E488V.vIGF2.WPRE.rBG (SEQ ID NO: 7);
(iv) CB7.CI.hSGSHcoV1.rBG (SEQ ID NO: 10);
(v) CB7.CI.hSGSHcoV1-4xmiR183.rBG (SEQ ID NO: 13);
(vi) CB7.CI.BIP.hSGSHcov1-A482Y_E488V.vIGF2.WPRE.4xmiR182.4xmiR183.rBG (SEQ ID NO: 37); or
(vii) CB7.CI.BIP.hSGSHcov1-A482Y_E488V.vIGF2.WPRE.4xmiR182.rBG (SEQ ID NO: 40).

The vector genome contains an AAV 5′ inverted terminal repeat (ITR) and an AAV 3′ ITR at the extreme 5′ and 3′ end, respectively. The ITRs flank the sequences of the expression cassette packaged into the AAV capsid which have sequences encoding a hSGSH, hSGSH.A482Y.E488V, BIP-hSGSH, or BIP-hSGSH.A482Y.E488V, in which BIP refers to ab exogenous signal peptide. The expression cassette further comprises regulatory sequences operably linked to the fusion protein coding sequences, including a CB7 promoter, chicken beta actin intron, rabbit beta-globin polyA, and optionally a WPRE element, which is a mutated WPRE element. See also, Kingsman et al., 2005 and Zanta-Boussif et al., 2009.

### Example 2. MPS IIIA Vector Screening Study MPS IIIA Mice

Next, we examined efficacy of SGSH constructs in MPSIIIA KO mice. In this study, we used hSGSH coV1 engineered construct based on the results observed in EXAMPLE 1 (WT mice expression study). AAVhu68 capsid was used for delivery of hSGSHcoV1 construct in MPSIIA and WT (control) mice (N-110 KO mice and N=10 WT mice). The AAVhu68.hSGSH was administered via unilateral ICV (direct intraventricular) injection at doses of 1×10¹⁰ (low dose) and 5×10¹⁰ (high dose). Mice were 2-3 months of age at a time of ICV injection. The duration of the study is 1 month. Furthermore, we examined multiple variations of the engineered hSGSHcoV1construct, including those comprising BiP signal peptide, stabilization amino acid (AA) changes, and/or vIGF2 peptide. The study endpoints used were lysosomal compartment size reduction (examined via LAMP1 immunofluorescence (IF) analysis), storage reduction (examined via GAG storage HS and MS), and SGSH expression levels (examined via enzyme activity assays and immunohistochemical analysis).

| | Group | | | | | Dose |
|---|---|---|---|---|---|---|
| Cohort | # | N | Gender | Genotype | Treatment | (GC) |
| 1 | 1 | 5/5 | M/F | KO | AAVhu68.CB7.hSGSHcoV1 | 1 × 10¹⁰ |
| | | | | | | (1e10) |
| | 2 | 5/5 | M/F | KO | AAVhu68.CB7.BIP-hSGSHcoV1 | (1e10) |
| | 3 | 5/5 | M/F | KO | AAVhu68.CB7.BIP-hSGSHcoV1- | (1e10) |
| | | | | | vIGF2 | |
| | 4 | 5/5 | M/F | KO | AAVhu68.CB7.BIP-hSGSH | (1e10) |
| | | | | | (A482Y E488V) coV1 | |
| | 5 | 5/5 | M/F | KO | AAVhu68.CB7.BIP-hSGSH | (1e10) |
| | | | | | (A482Y E488V) coV1 - vIGF2 | |
| | 6 | 5/5 | M/F | KO | PBS | n/a |
| | 7 | 5/5 | M/F | WT | PBS | n/a |
| 2 | 8 | 5/5 | M/F | KO | AAVhu68.CB7.hSGSHcoV1 | (5e10) |
| | 9 | 5/5 | M/F | KO | AAVhu68.CB7.BIP-hSGSHcoV1 | (5e10) |
| | 10 | 5/5 | M/F | KO | AAVhu68.CB7.BIP-hSGSHcoV1- | (5e10) |
| | | | | | vIGF2 | |
| | 11 | 5/5 | M/F | KO | AAVhu68.CB7.BIP-hSGSH | (5e10) |
| | | | | | (A482Y E488V) coV1 | |
| | 12 | 5/5 | M/F | KO | AAVhu68.CB7.BIP-hSGSH | (5e10) |
| | | | | | (A482Y E488V) coV1 - vIGF2 | |

In cohort 1, the collected tissue samples include brain, spinal cord and liver. In cohort 2, the collected tissue samples include heart and spleen.

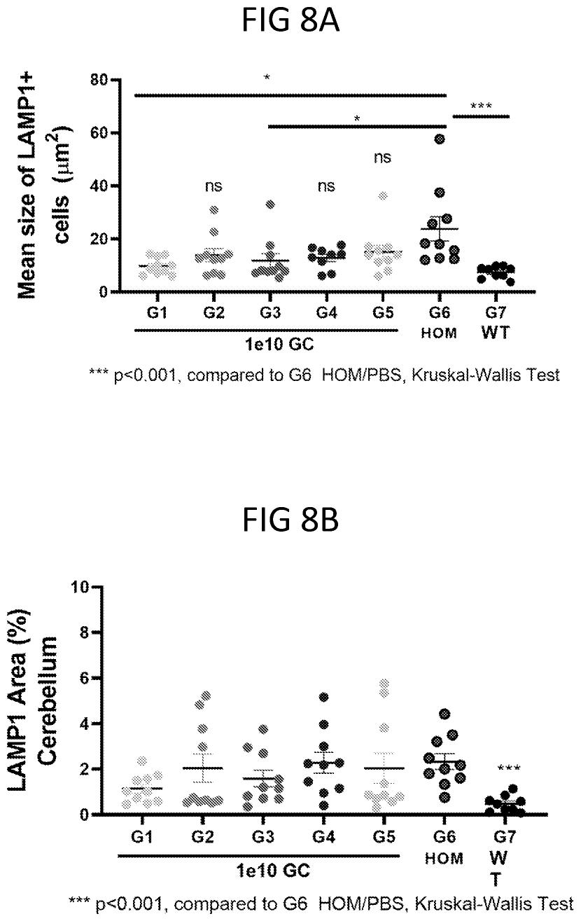

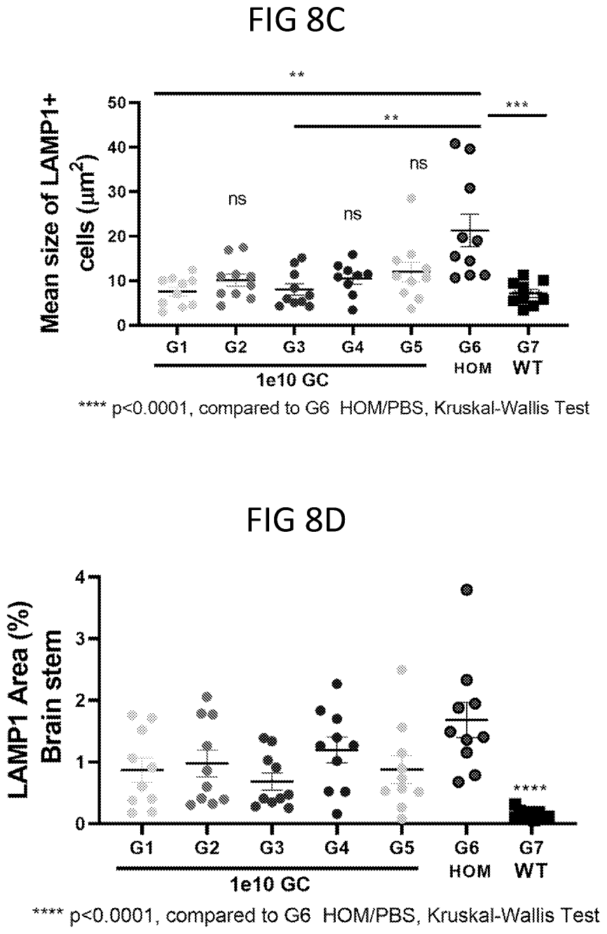

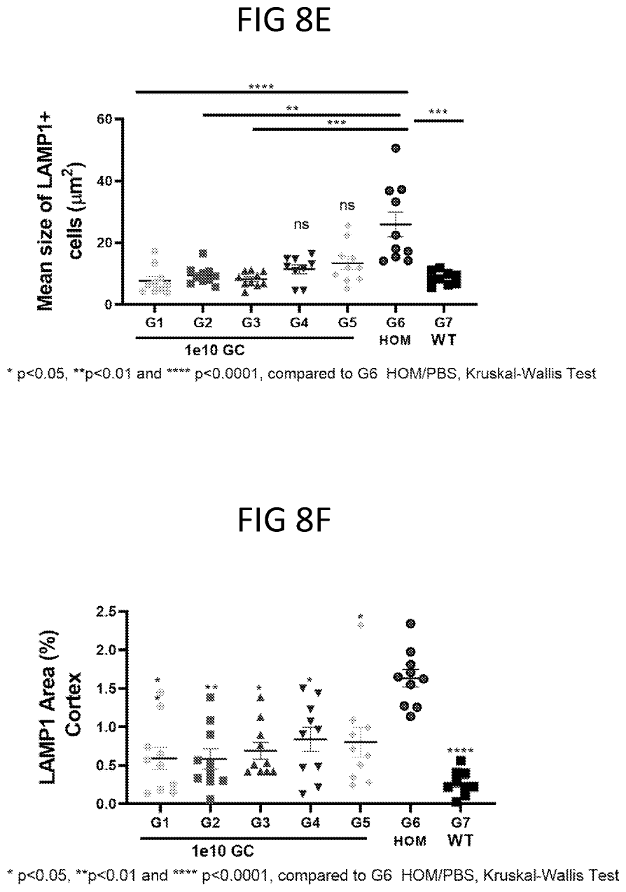

FIGS. 8A to 8F shows endpoint analysis of lysosomal compartment reduction as examined via LAMP1 quantification using immunofluorescent and histochemical analysis in MPS IIIA SGSH KO mice administered at a low dose (1×10¹⁰) of AAVhu68.hSGSH. Group 1 (G1)=AAVhu68.CB7.hSGSHcoV1; G2=AAVhu68.CB7.BIP-hSGSHcoV1; G3=AAVhu68.CB7.BIP-hSGSHcoV1-vIGF2; G4=AAVhu68.CB7.BIP-hSGSH (A482Y E488V) coV1; G5=AAVhu68.CB7.BIP-hSGSH (A482Y E488V) coV1-vIGF2; G6=KO mice PBS controls; G7=WT mice PBS controls. FIG. 8A shows mean size of LAMP1-positive cells (μm²) in cerebellum. FIG. 8B shows percent LAMP1-area in cerebellum. FIG. 8C shows mean size of LAMP1-positive cells (μm²) in brain stem. FIG. 8D shows percent LAMP1-area in brain stem. FIG. 8E shows mean size of LAMP1-positive cells (μm²) in cortex. FIG. 8F shows percent LAMP1-area in cortex.

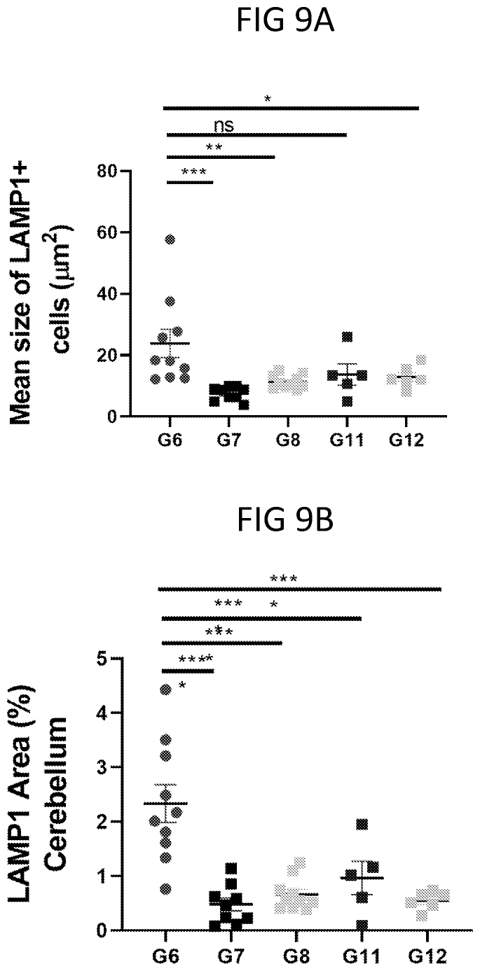

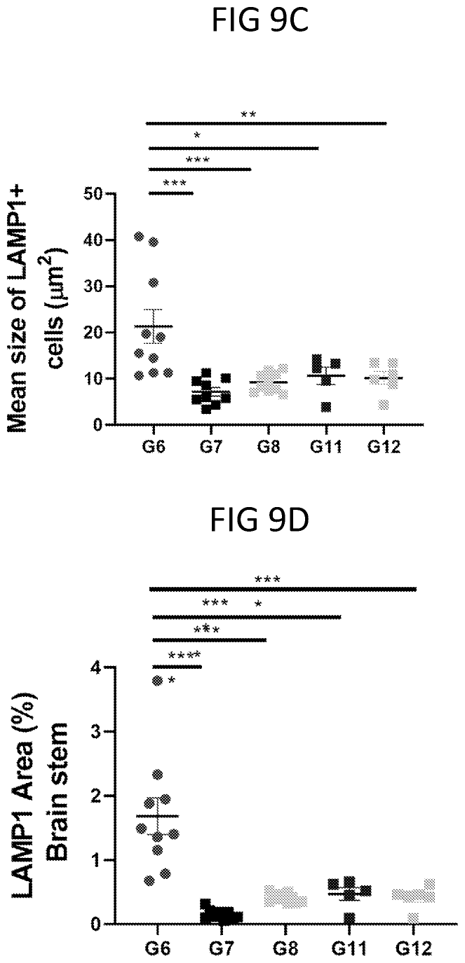

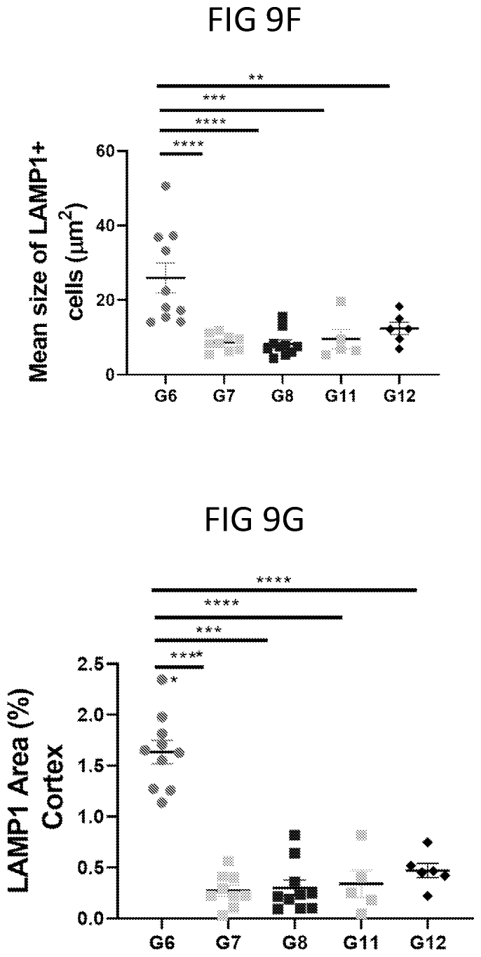

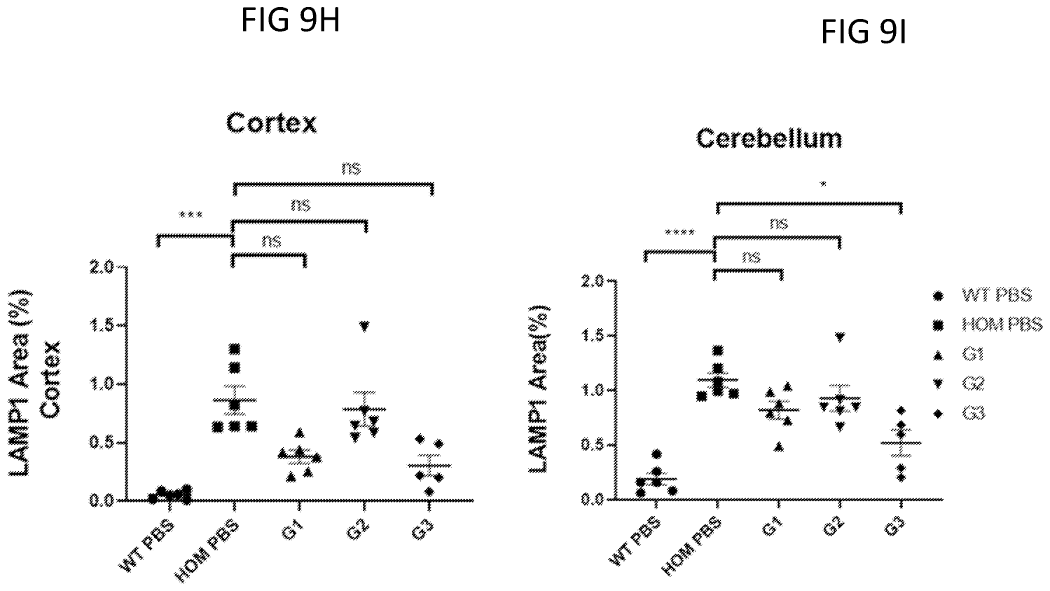

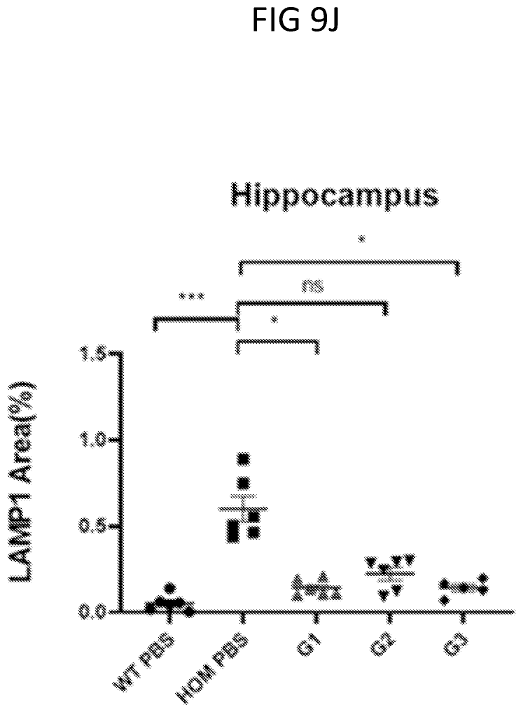

FIGS. 9A to 9J shows endpoint analysis of lysosomal compartment reduction as examined via LAMP1 quantification using immunofluorescent and histochemical analysis in MPS IIIA SGSH KO mice administered at a high dose (1×10¹¹) of AAVhu68.hSGSH. G6 and G7 similar to FIG. 8 (KO and WT PBS controls); G8=AAVhu68.CB7.hSGSHcoV1; G11=AAVhu68.CB7.BIP-hSGSH (A482Y E488V); G12=AAVhu68.CB7.BIP-hSGSH (A482Y E488V) coV1-vIGF2.

FIG. 9A shows mean size of LAMP1-positive cells (μm²) in cerebellum. FIG. 9B shows percent LAMP1-area in cerebellum. FIG. 9C shows mean size of LAMP1-positive cells (μm²) in brain stem. FIG. 9D shows percent LAMP1-area in brain stem. FIG. 9E shows mean size of LAMP1-positive cells (μm²) in cortex. FIG. 9F shows percent LAMP1-area in cortex. FIG. 9H shows percent LAMP1-area in cortex for G1, G2 and G3, as defined in FIGS. 8A-8F, at a dose of 2×10¹⁰ GC in 2-3 month old mice, measured at 1 month using the Mann-Whitney test. FIG. 9I shows percent LAMP-1 area in cerebellum for G1, G2 and G3, at a dose of 2×10¹⁰ GC in 2-3 month old mice, measured at 1 month using the Mann-Whitney test. FIG. 9J shows percent LAMP-1 area in hippocampus for G1, G2 and G3, at a dose of 2×10¹⁰ GC in 2-3 month old mice, measured at 1 month using the Mann-Whitney test. These results show that close to the injection site, similar performance of WT (group 1) and engineered-WPRE (group 3) candidates was observed, and in the cerebellum, where no SGSH was detected by IHC regardless of the group, only the engineered-WPRE candidate demonstrated clearance of LAMP1 positive material.

Next, the following assays were further performed: SGSH enzyme activity, glycosaminoglycan (GAG) (HS) accumulation/reduction, and Anti-SGSH antibody titer. The SGSH enzyme activity was examined using Liquid Chromatography with tandem mass spectrometry (LC-MS/MS) quantitation of product formation from one-step reaction using 2-Naphthalene-GlcNS. GAG (HS) accumulation/reduction was examined using LC-MS/MS quantitation of disaccharide breakdown products from butanolysis of HS in tissues. Anti-SGSH antibody titer was examined using ELISA.

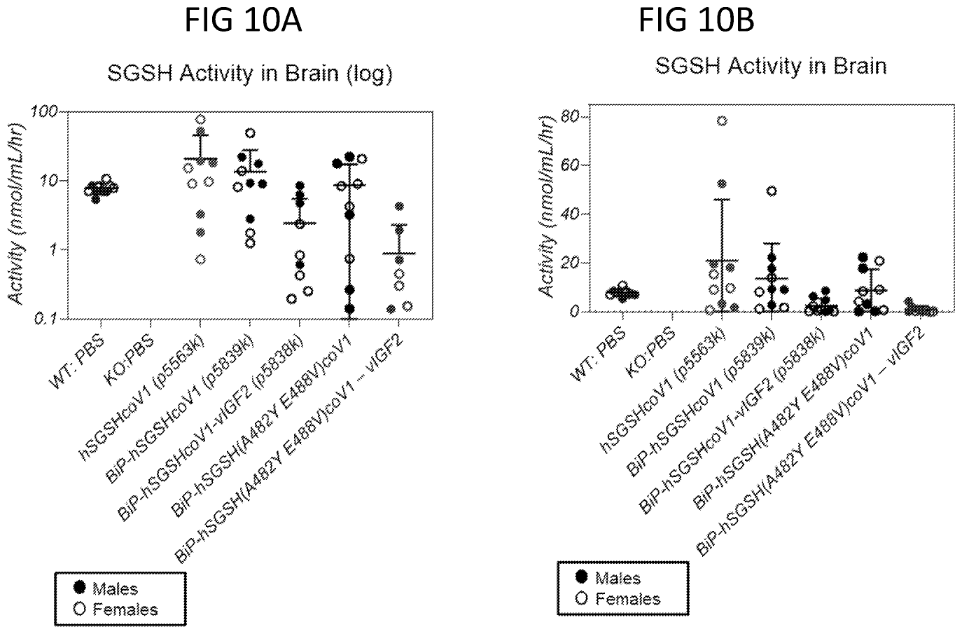

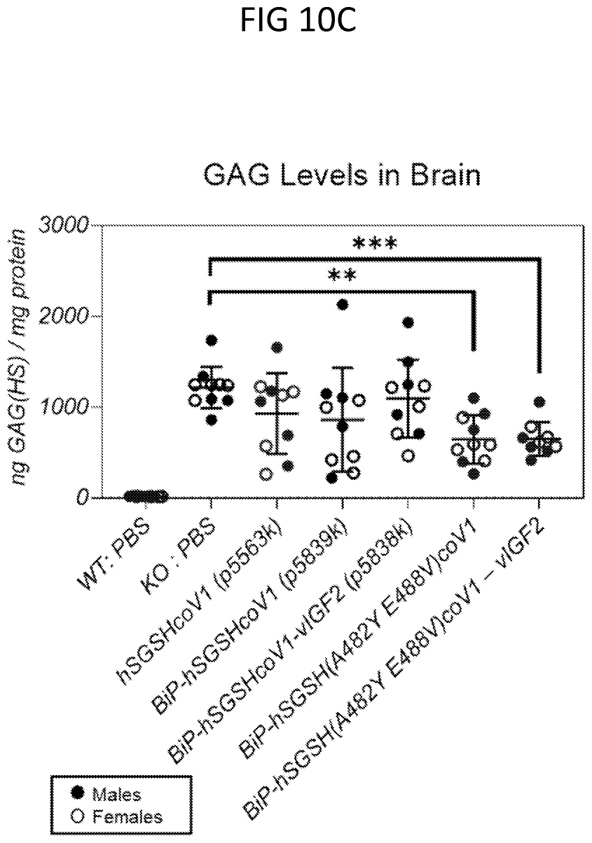

FIGS. 10A to 10C shows SGSH activity and GAG reduction in brain of male and female mice. FIG. 10A shows SGSH activity in brain plotted as activity (nmol/mL/hr). FIG. 10B shows SGSH activity in brain, plotted as log activity. FIG. 10C shows GAG levels in brain plotted as ng GAG(HS) per mg protein.

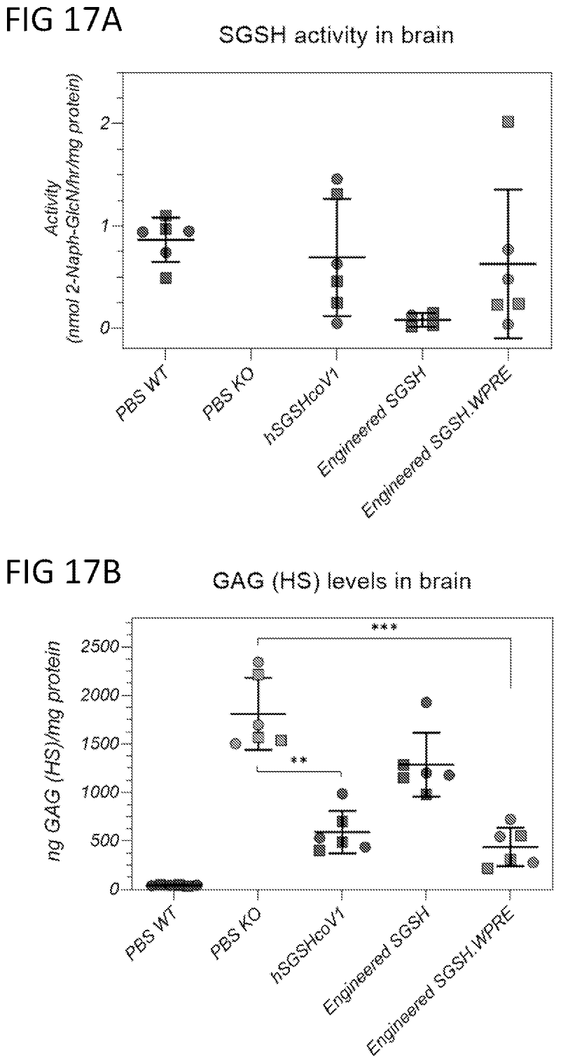

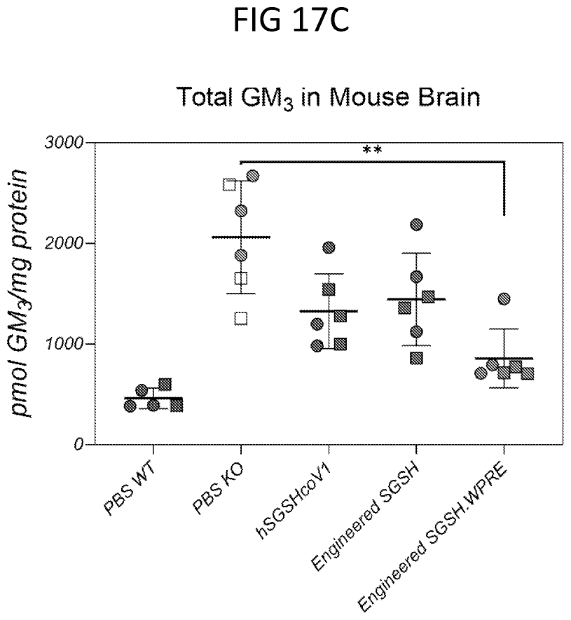

Additionally, we examined SGSH activity and substrate reduction in treated mouse brain after 1 month following administration. Briefly, mice were administered a dose of 2e10 (1×10¹² vg/kg) of rAAV as indicated (hSGSHcoV1, coV1BIP-hSGSH(A482Y E488V)coV1-vIGF2, and coV1BIP-hSGSH(A482Y E488V)coV1-vIGF2 with WPRE). FIG. 17A shows SGSH activity in treated mouse brain. FIG. 17B shows GAG (HS) levels in brain. FIG. 17C shows total GM3 in mouse brain. These results show that the addition of WPRE to the engineered candidate rescues expression to levels similar to the WT construct and allows comparable or better storage clearance.

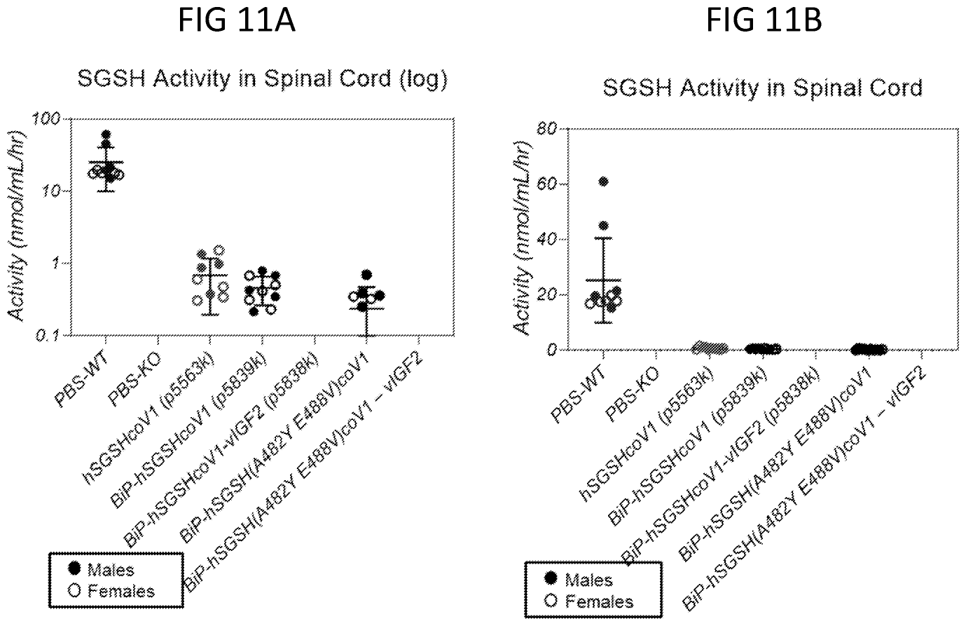

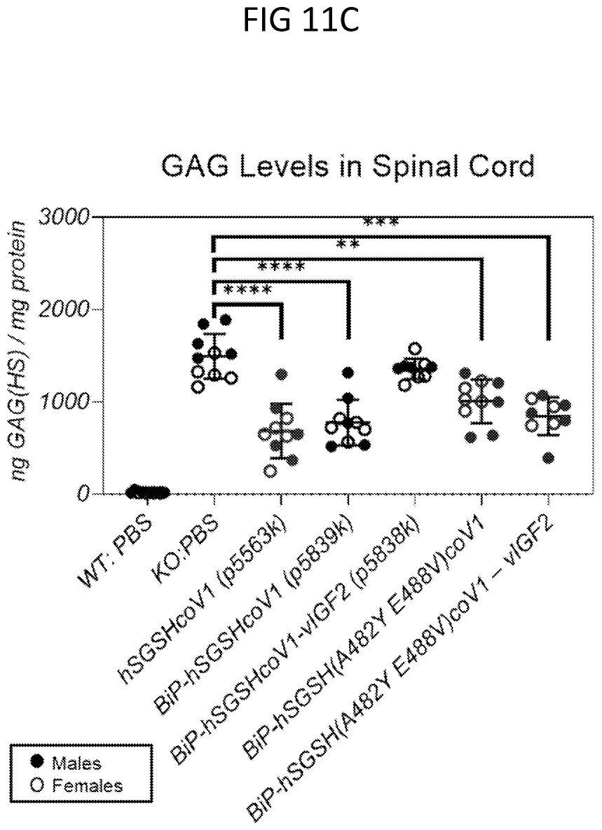

FIGS. 11A to 11C shows SGSH activity in GAG reduction in spinal cord of male and female mice. FIG. 11A shows SGSH activity in spinal cord plotted as activity (nmol/mL/hr). FIG. 11B shows SGSH activity in spinal cord plotted as log activity. FIG. 11C shows GAG levels in spinal cord plotted as ng GAG(HS) per mg protein.

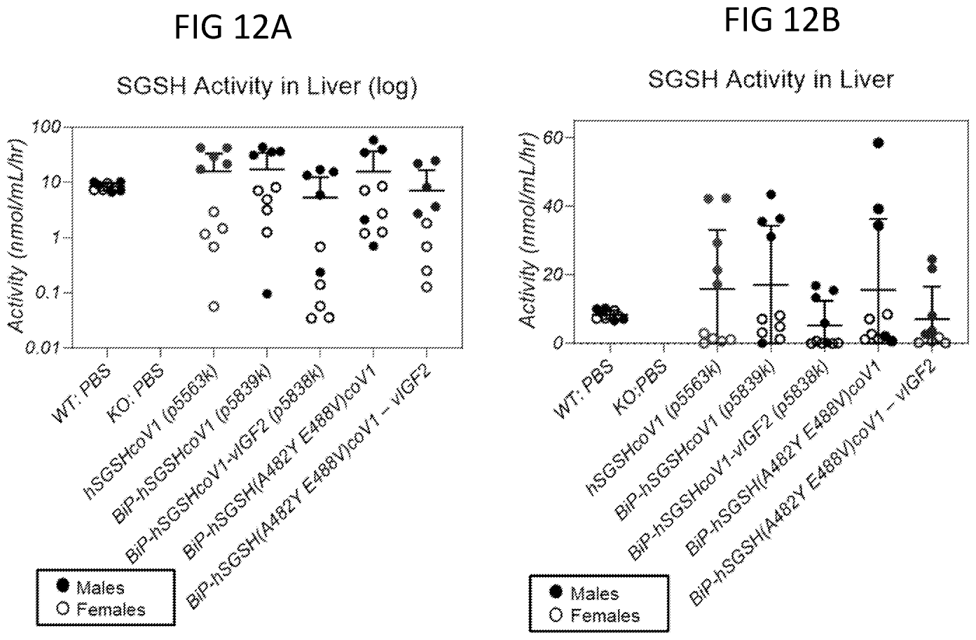

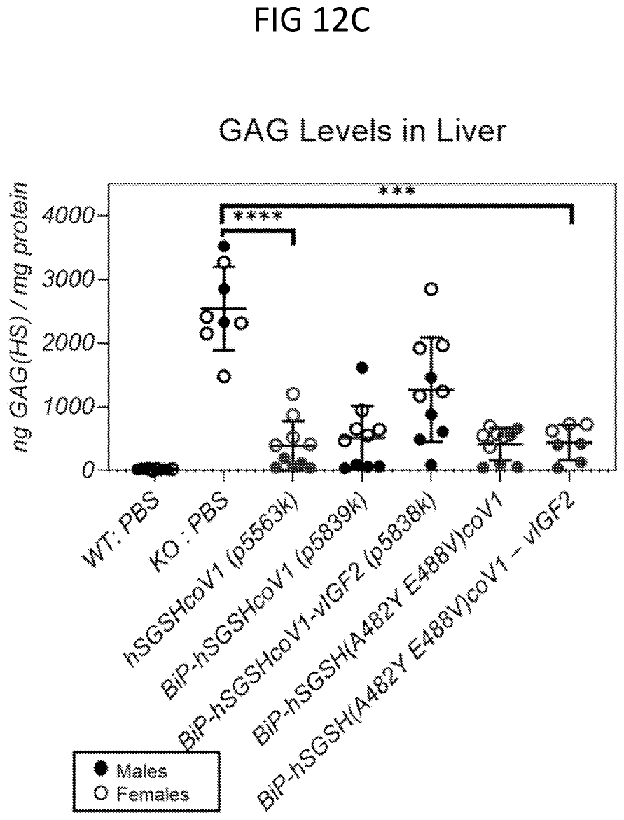

FIGS. 12A to 12C shows SGSH activity in GAG reduction in liver of male and female mice. FIG. 12A shows SGSH activity in liver plotted as activity (nmol/mL/hr). FIG. 12B shows SGSH activity in liver plotted as log activity. FIG. 12C shows GAG levels in liver plotted as ng GAG(HS) per mg protein.

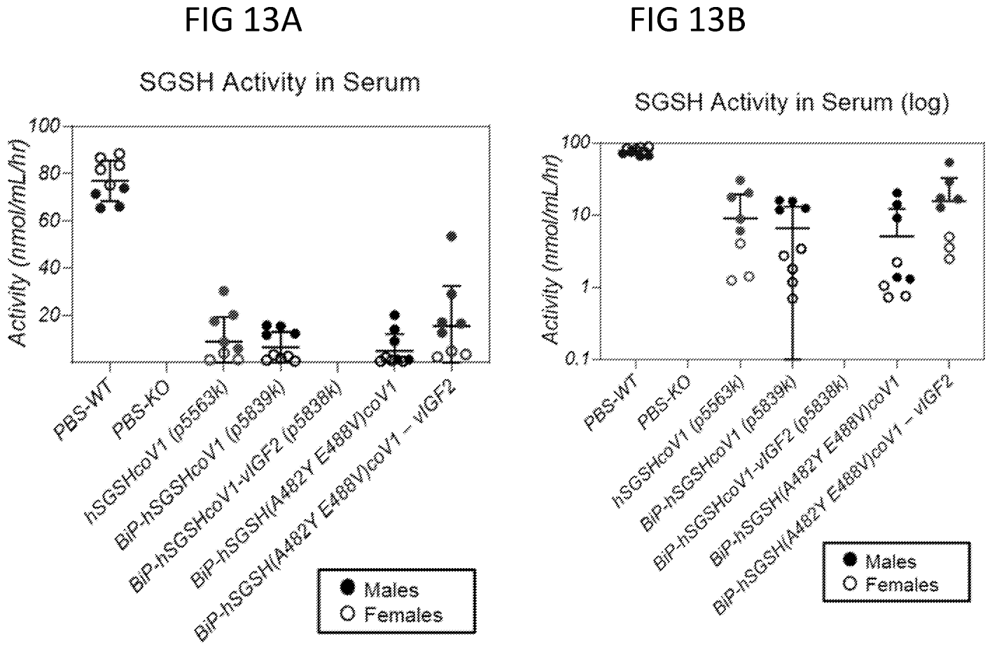

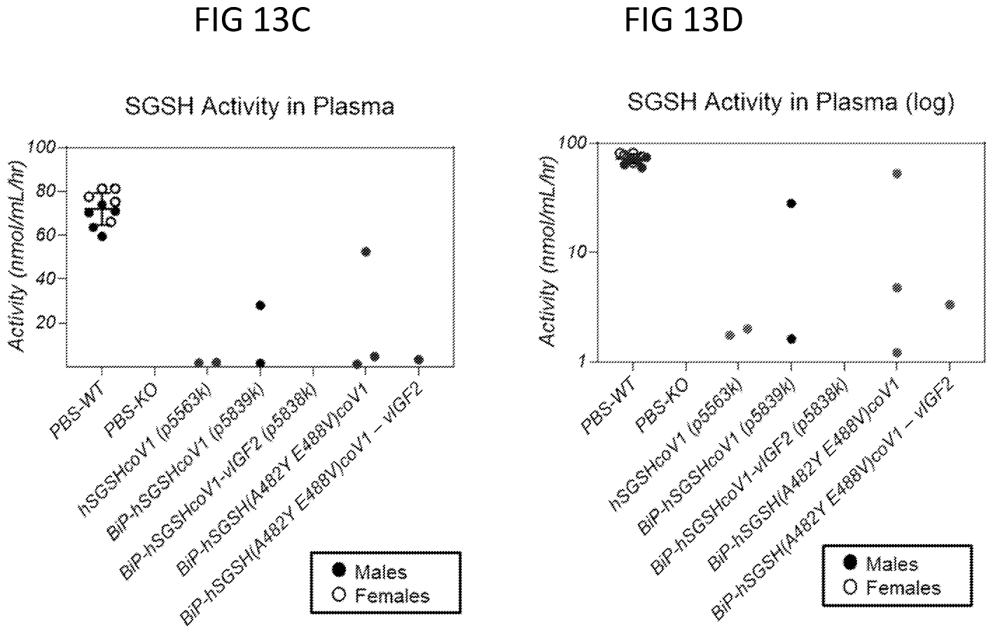

FIG. 13A shows SGSH activity in serum plotted as activity (nmol/mL/hr) as measure on day 7 of the post ICV injection. FIG. 13B shows SGSH activity in serum plotted as log activity (nmol/mL/hr) on day 7 post ICV injection. FIG. 13C shows SGSH levels activity in plasma plotted as activity (nmol/mL/hr) as measured at one month post ICV injection. FIG. 13D shows SGSH levels activity in plasma plotted as log activity (nmol/mL/hr) as measured at one month post ICV injection.

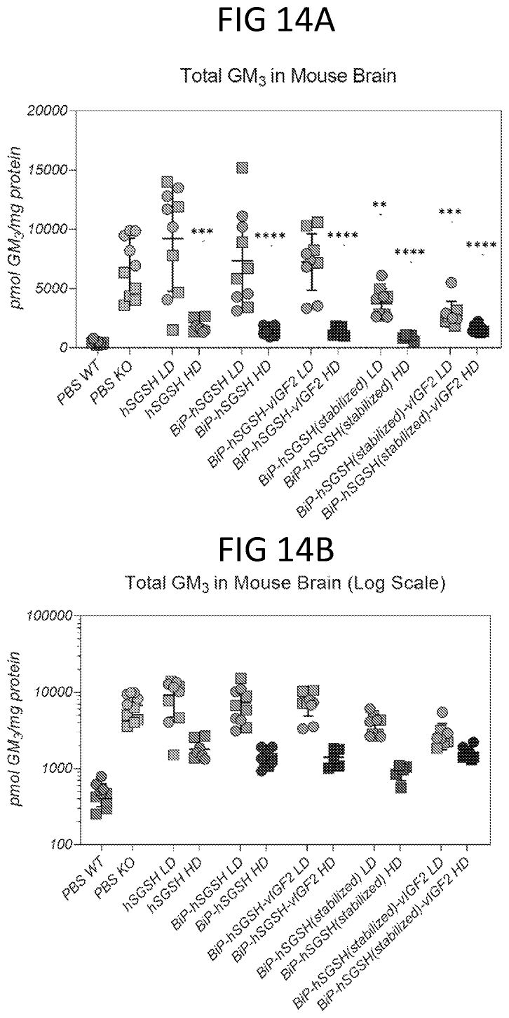

Additionally, we examined GM3 levels in mouse brain samples. FIG. 14A shows levels of total GM3 in mouse brain plotted as pmol GM3 per mg protein. FIG. 14B shows levels of total GM3 in mouse brain potted as log scale pmol GM3 per mg protein. These results show that at low dose engineered constructs (BIP-stabilized AA SGSH-vIGF2) performed superior to others.

In summary, we examined and compared engineered hSGSH-coV1 construct with four (4) other engineered SGSH candidates. The SGSH activity was observed highest with hSGSH-coV1 construct. Additionally, we observed that GAG reduction was equally reduced with two (2) engineered versions: coV1BIP-hSGSH(A482Y E488V)coV1 and coV1BIP-hSGSH(A482Y E488V)coV1-vIGF2. Furthermore, we observed that GM3 reduction was superior at low dose with coV1BIP-hSGSH(A482Y E488V)coV1-vIGF2. All candidates reduced LAMP1 staining compared with MPSIIIA KO mice at high dose. At low dose, variable results were observed due to poor expression of engineered constructs. In view of the above mentioned results, further studies focus on BIP-hSGSH(A482Y_E488Y)coV1-vIGF2 and hSGSHcoV1 (non-engineered), which is used as control.

### Example 3. MPS IIIA Vector Study in Mice and Non-Human Primates (NHPs)

Next, we further examine the efficacy of AAV.hSGSH comprising engineered SGSH coding sequence (BIP-hSGSH(A482Y_E488Y)coV1-vIGF).

First in this study, we examine SGSH expression and enzyme activity in engineered construct (BIP-hSGSH(A482Y_E488Y)coV1-vIGF) comprising WPRE element in the vector genome of AAV (see also, Example 1). Additionally, we examine the effect of addition of miR detargeting sites and evaluate the impact on efficacy. The miR detargeting is performed with addition of 4 or more of miRNA targeting sequences, i.e., miR182 and miR 183.

Next, we examine and compare biodistribution and transduction levels of AAV.hSGSH when administered using AAVhu68 and AAVrh91 capsids in non-human primates (NHPs). We further examine and compare transduction levels of AAV.hSGSH.WPRE when AAVhu68 capsid. Additionally, we examine safety, efficacy and evaluate impact of AAV.hSGSH which further comprise DRG detargeting miRNA target sequences. Table immediately below shows a summarized layout of the NHP study. Briefly, total of 15 NHPs (N-3/group) are administered AAV via ICM (intra-cisterna magna) at a dose of 3×10¹³ GC (adjusted based on rhesus macaques brain weight (e.g., 90 g)). The primary readouts of the study are pharmacology (SGSH expression levels), toxicology, histopathology.

| Group # | Treatment |
|---|---|
| 1 | AAVhu68.CB7.hSGSHcoV1 |
| 2 | AAVrh91.CB7.hSGSHcoV1 |
| 3 | AAVhu68.CB7.hSGSHcoV1. miR |
| 4 | AAVhu68.CB7.BIP-hSGSH (A482Y |
| | E488V) coV1 - vIGF2.WPRE |
| 5 | AAVhu68.CB7.BIP-hSGSH (A482Y |
| | E488V) coV1 - vIGF2.WPRE.miR |

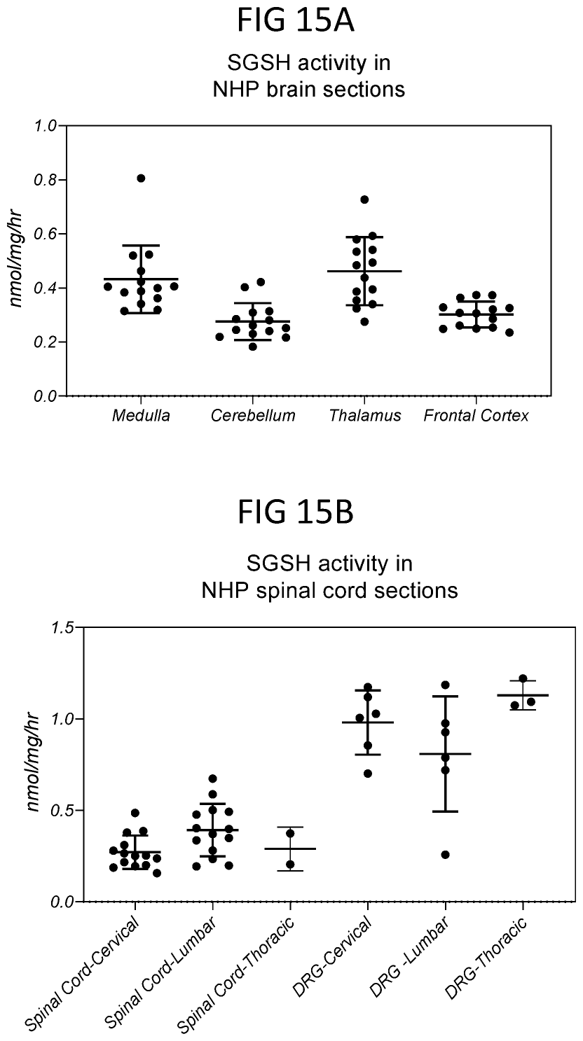

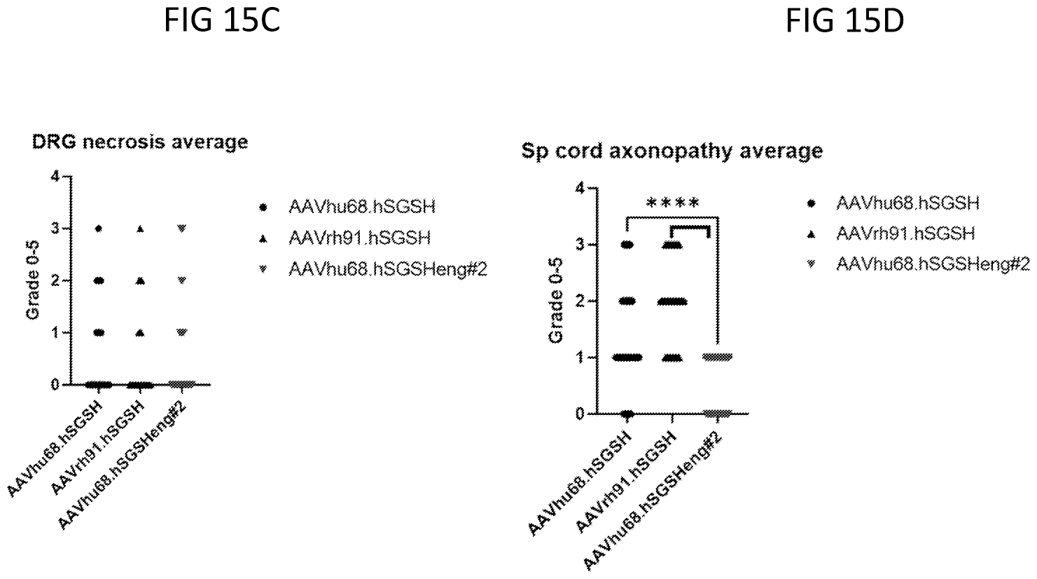

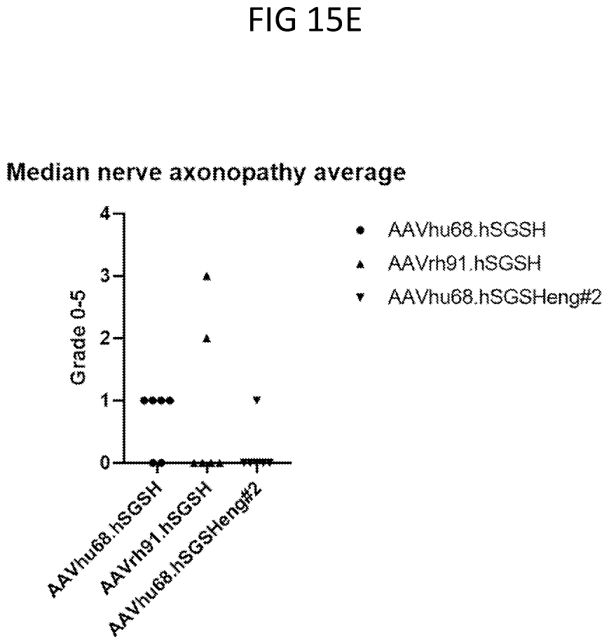

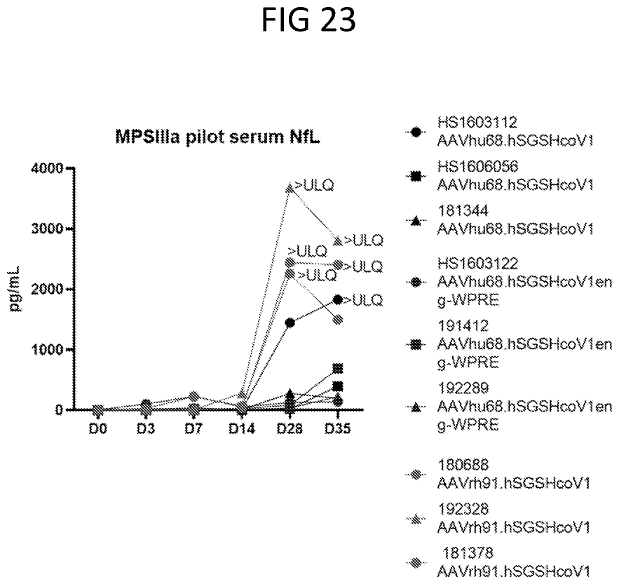

Preliminary studies were performed to examine and measure baseline SGSH activity in untreated rhesus macaques (NHPs) prior to AAV.hSGSH vector administration. FIG. 15A shows levels of SGSH baseline activity in untreated NHP brain sections plotted as nmol/mg/hr, as measured in medulla, cerebellum, thalamus, frontal cortex. FIG. 15B shows levels of SGSH baseline activity in untreated NHP spinal cord sections plotted as nmol/mg/hr, as measured in spinal cord cervical, thoracic, lumbar, and dorsal root ganglion (DRG) cervical, lumbar and thoracic sections. These data show a significant SGSH activity observed in DRG sections and plasma. FIG. 15B shows levels of SGSH baseline activity in untreated NHP spinal cord sections plotted as nmol/mg/hr, as measured in spinal cord cervical, thoracic, lumbar, and dorsal root ganglion (DRG) cervical, lumbar and thoracic sections. See. FIGS. 15C-15E. FIG. 23 shows serum Nfl in treated NHP, which shows that Serum NfL aligns with histopathology axonopathy severity.

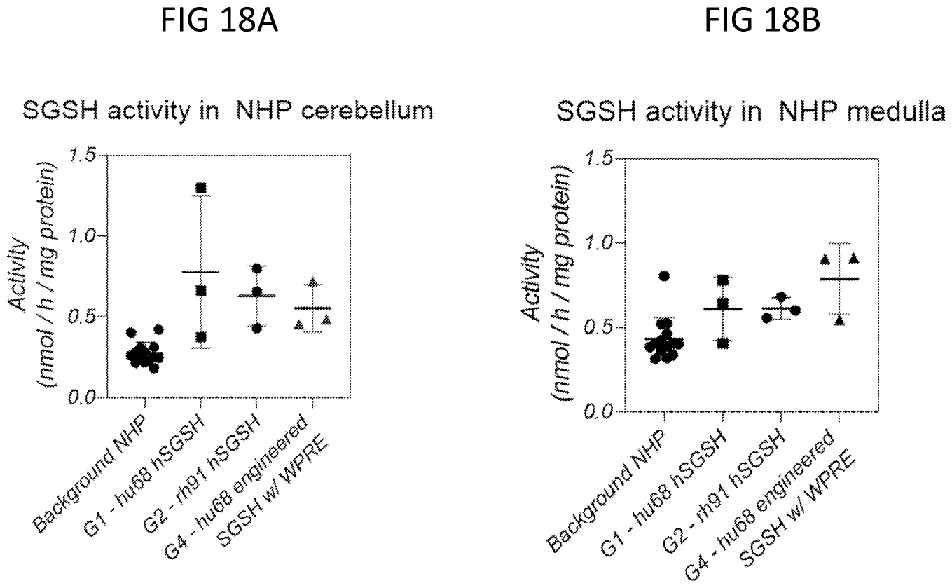

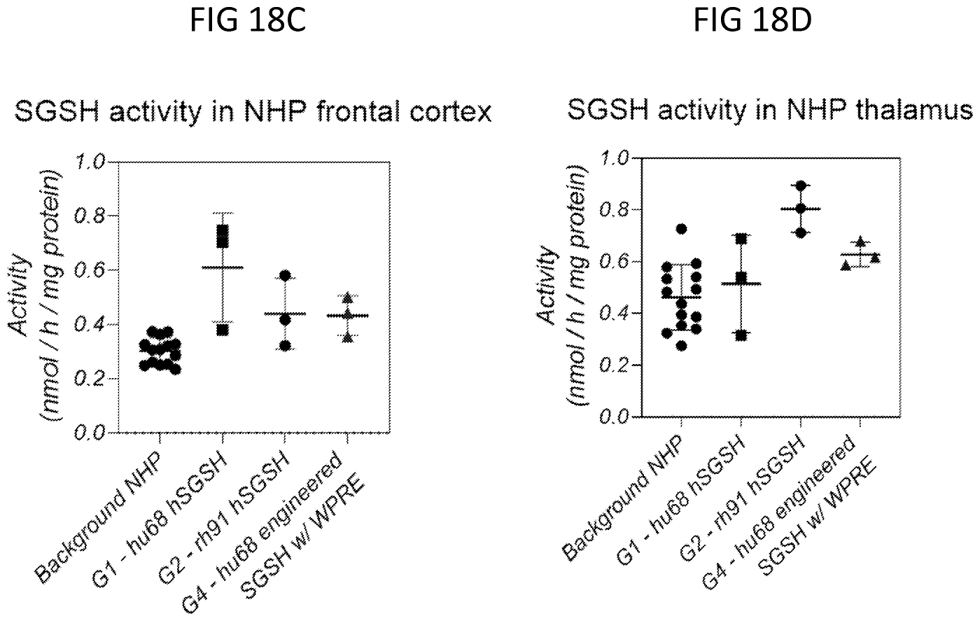

FIG. 18A shows SGSH activity in treated NHP cerebellum brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 18B shows SGSH activity in treated NHP medulla brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 18C shows SGSH activity in treated NHP frontal cortex brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 18D shows SGSH activity in treated NHP thalamus brain tissue, compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE).

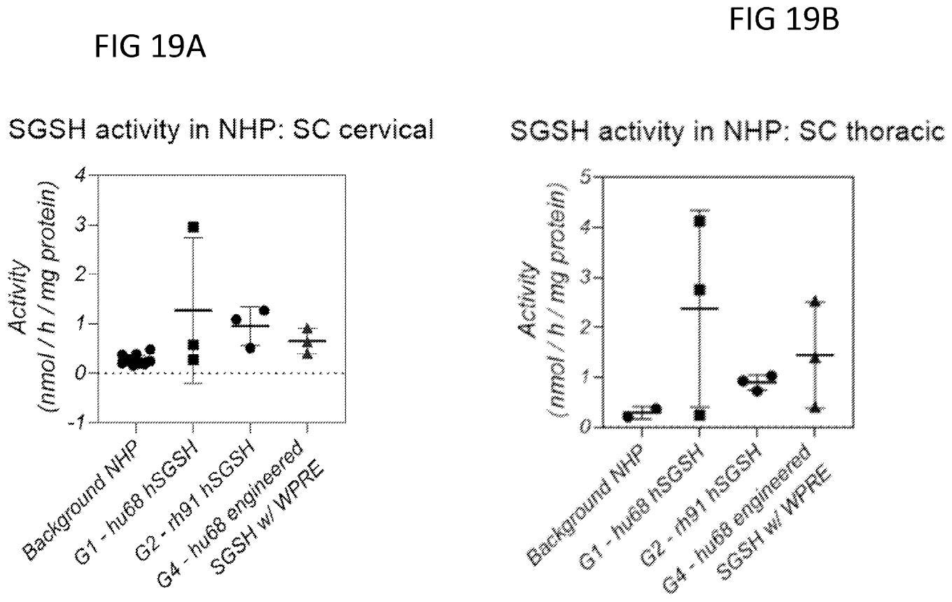

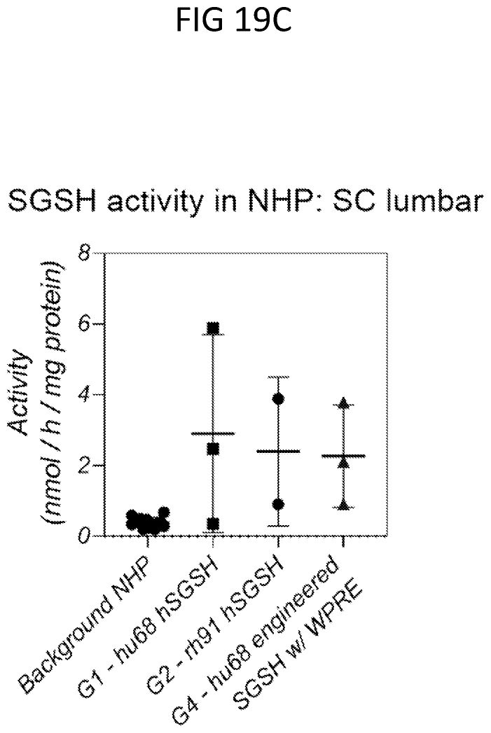

FIG. 19A shows SGSH activity in treated NHP spinal cord section (SC cervical), compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 19B shows SGSH activity in treated NHP spinal cord section (SC thoracic), compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE). FIG. 19C shows SGSH activity in treated NHP spinal cord section (SC lumbar), compared against background for G1 (AAVhu68.CB7.hSGSHcoV1), G2 (AAVrh91.CB7.hSGSHcoV1), and G4 (AAVhu68.CB7.BIP-hSGSH (A482Y E488V) coV1-vIGF2.WPRE).

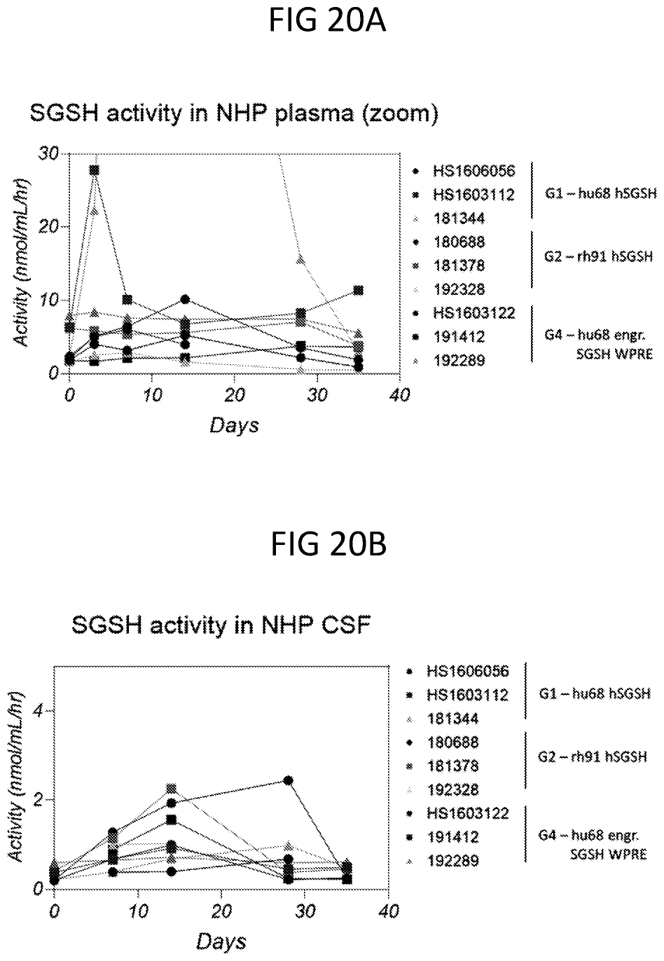

FIG. 20A shows SGSH activity in treated NHP plasma. FIG. 20B shows SGSH activity in treated CSF

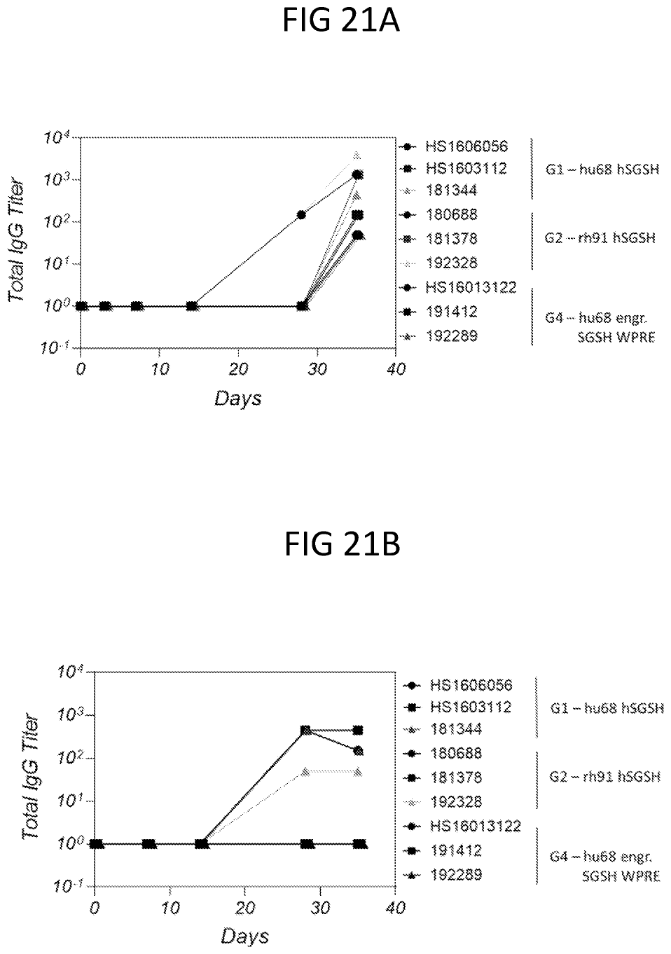

FIG. 21A shows total anti-hSGSH IgG titer in NHP plasma. FIG. 21B shows total anto-hSGSH titer in CSF. These results show that Group 2 (rAAVrh91.hGSH) construct is more immunogenic in CSF and plasma than group 1 (AAVhu68.CB7.hSGSHcoV1) or 4 (AAVhu68.CB7.BIP-hSGSH (A482Y E488V) coV1-vIGF2.WPRE) constructs.

Additionally, we performed ELISPOT for samples obtained from NHP treated with G1 (AAVhu68.CB7.hSGSHcoV1; Table A) and G4 (AAVhu68.CB7.BiP-hSGSH (A482Y E48 8V) coV1-vIGF2.WPRE; Table B), results for which are described in tables below.

TABLE A

| | | | | | | | | | | SFU per | SFU per |
|---|---|---|---|---|---|---|---|---|---|---|---|
| | | | | | | | | | | 2.0E+04 | 2.0E+03 |
| | | DMSO | SFU million cells | | | | | | | cells | cells |
| | | No | AAVhu68 | | | hSGSHcoV1 | | | Positive controls | | |
| Sample | Lymphocyte | Stim. | A | B | C | A | B | C | PHA | CD3 | PMA/ION |
| HS1606056 | Baseline | 15 | 8 | 25 | 10 | 13 | 20 | 20 | TNTC | 52 | 134 |
| HS1606056 | Day 34 | 18 | 18 | 43 | 55 | 143 | 95 | 10 | TNTC | 10 | 111 |
| HS1606056 | Day 25 | 8 | 8 | 13 | 5 | 70 | 50 | 10 | TNTC | 8 | 123 |
| | Spleen | | | | | | | | | | |
| HS1606056 | Day 35 | 13 | 3 | 13 | 18 | 45 | 28 | 5 | 820* | 2 | 58 |
| | Cervical LN | | | | | | | | | | |
| HS1606056 | Day 35 | 153 | 175 | 185 | 178 | 588 | 390 | 160 | TNTC | 72 | TNTC |
| | Liver | | | | | | | | | | |
| HS1603112 | Baseline | 28 | 18 | 23 | 10 | 18 | 10 | 3 | TNTC | 25 | 34 |
| HS1603112 | Day 34 | 13 | 18 | 13 | 20 | 200 | 303 | 23 | TNTC | 7 | 97 |
| HS1603112 | Day 25 | 28 | 20 | 10 | 18 | 115 | 178 | 13 | TNTC | 6 | 131 |
| | Spleen | | | | | | | | | | |
| HS1603112 | Day 35 | 0* | X | X | X | X | X | X | X | 0 | 3 |
| | Cervical LN | | | | | | | | | | |
| HS1603112 | Day 35 | 233 | 255 | 240 | 143 | 1338 | 1653 | 213 | TNTC | 47 | TNTC |
| | Liver | | | | | | | | | | |
| 181344 | Baseline | 5 | 8 | 23 | 20 | 20 | 15 | 18 | TNTC | 6 | 195 |
| 181344 | Day 34 | 25 | 113 | 170 | 163 | 203 | 200 | 20 | TNTC | 2 | 240 |
| 181344 | Day 25 | 135 | 320 | 293 | 283 | 313 | 248 | 85 | TNTC | 3 | 202 |
| | Spleen | | | | | | | | | | |
| 181344 | Day 35 | 0 | 5 | 33 | 10 | 15 | 13 | 3 | 348 | 0 | 118 |
| | Cervical LN | | | | | | | | | | |
| 181344 | Day 35 | 23 | 228 | 1145 | 1273 | 888 | 723 | 25 | TNTC | 57 | TNTC |
| | Liver | | | | | | | | | | |

TABLE B

| | | DMSO | | | | | | | | | |
|---|---|---|---|---|---|---|---|---|---|---|---|
| | | No | AAVhu68 | | | hSGSHcoV1 | | | Positive controls | | |
| Sample | Lymphocyte | Stim. | A | B | C | A | B | C | PHA | CD3 | PMA/ION |
| HS1603122 | Baseline | 13 | 18 | 13 | 8 | 13 | 8 | 5 | TNTC | 9 | 87 |
| HS1603122 | Day 34 | 5 | 53 | 25 | 43 | 243 | 805 | 48 | TNTC | 7 | 129 |
| HS1603122 | Day 25 | 38 | 93 | 110 | 108 | 263 | 1190 | 90 | TNTC | 16 | 122 |
| | Spleen | | | | | | | | | | |
| HS1603122 | Day 35 | 0 | 5* | 5* | 5* | 10* | 20* | 0* | 385* | 8 | 1 |
| | Cervical LN | | | | | | | | | | |
| HS1603122 | Day 35 | 258 | 425 | 263 | 248 | 1423 | 2093 | 328 | TNTC | 80 | TNTC |
| | Liver | | | | | | | | | | |
| 192289 | Baseline | 3 | 0 | 0 | 0 | 3 | 0 | 3 | 1268 | 3 | 79 |
| 192289 | Day 34 | 0 | 0 | 0 | 3 | 3 | 5 | 0 | 358 | 2 | 37 |
| 192289 | Day 25 | 25 | 10 | 48 | 15 | 40 | 70 | 35 | 933 | 7 | 92 |
| | Spleen | | | | | | | | | | |
| 192289 | Day 35 | 0* | 0* | 0* | 0* | X | X | X | X | 0 | 2 |
| | Cervical LN | | | | | | | | | | |
| 192289 | Day 35 | 215 | 185 | 298 | 203 | 295 | 560 | 365 | TNTC | 20 | TNTC |
| | Liver | | | | | | | | | | |
| 191412 | Baseline | 45 | 35 | 25 | 15 | 30 | 33 | 25 | TNTC | 38 | 55 |
| 191412 | Day 34 | 23 | 103 | 70 | 48 | 108 | 258 | 98 | TNTC | 55 | 79 |
| 191412 | Day 25 | 83 | 270 | 155 | 145 | 93 | 148 | 85 | TNTC | 36 | 172 |
| | Spleen | | | | | | | | | | |
| 191412 | Day 35 | 23 | 48 | 120 | 13 | 35 | 88 | 43 | 825 | 1 | 71 |
| | Cervical LN | | | | | | | | | | |
| 191412 | Day 35 | 83 | 195 | 313 | 110 | 118 | 343 | 203 | TNTC | 127 | TNTC |
| | Liver | | | | | | | | | | |

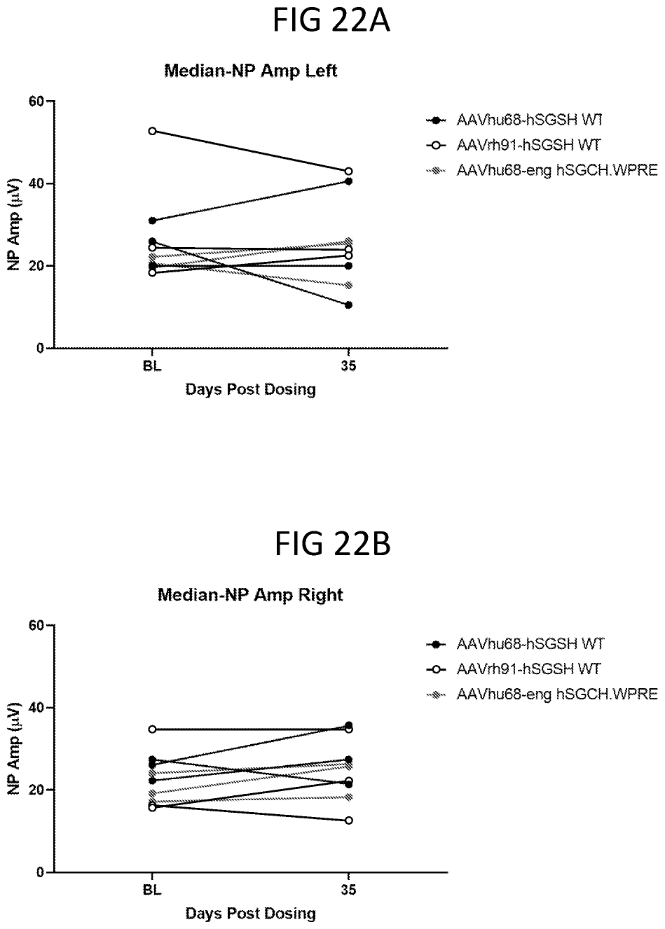

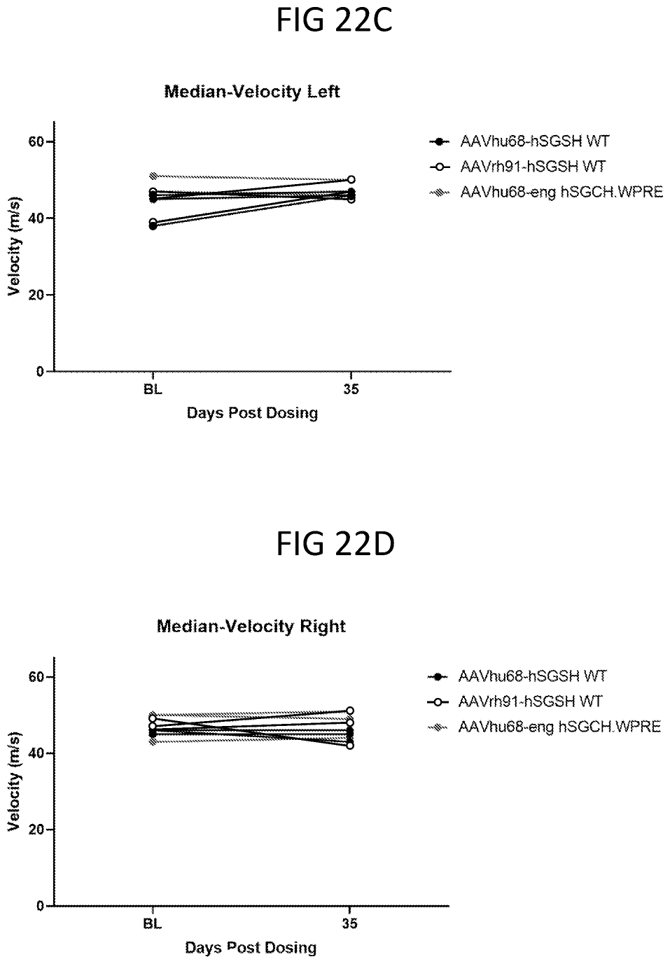

FIG. 22A shows results of the nerve conduction velocity (NCV), plotted as NP (nerve polarization in Amp) in left median nerve. FIG. 22B shows results of the nerve conduction velocity (NCV), plotted as NP (nerve polarization in Amp) in right median nerve. FIG. 22C shows results of the nerve conduction velocity (NCV), plotted as velocity in left median nerve. FIG. 22D shows results of the nerve conduction velocity (NCV), plotted as velocity in right median nerve. These results show that NP or velocity in median nerves did not show significant reduction with engineered-WPRE candidate.

In summary, the mouse bioanalytical studies showed that SGSH activity for all candidates was equivalent to untreated WT in brain and liver and generally indistinguishable from one another; substrate reduction was most reduced in engineered WPRE candidate. Brain lysosomal pathology correction by LAMP-1 IF was most reduced with engineered WPRE candidate at distance from injection site. Furthermore, the NHP pilot safety/pharmacology showed that the expression seems on par or better with the lead engineered-WPRE candidate. Engineered hSGSH-WPRE had a similar immunogenicity profile as non-engineered based on ELISPOT data. Engineered hSGSH-WPRE showed the best safety profile based on DRG toxicity biomarker (NfL) and histopathology.

### Example 4. AAV.hSGSH Efficacy in Mouse Neurospheres

In this study we examine and compare SGSH expression levels and efficacy of AAV.hSGSH comprising WT SGSH construct, engineered SGSH construct and engineered SGSH construct further comprising WPRE element in AAV vector genome. In this study, a 48-well plate seeded with WT neurospheres (these differentiate into an astrocytic lineage) is used, which are incubated for 10 days following AAV-treatment to allow for maximum expression. We used in cell western analysis (i.e., plate image), wherein 800 channel fluorescence (green) indicates trans gene expression via secondary antibody (anti-SGSH), and 700 channel fluorescence (red) indicates cell mass via the CellTag™ tool. Table immediately below shows schematic representation of the plate layout and the experimental conditions/treatment used.

| | Col. 1 | Col. 2 | Col. 3 | Col. 4 | Col. 5 | Col. 6 | Col. 7 | Col. 8 |
|---|---|---|---|---|---|---|---|---|
| Row 1: WT SGSH | | | | | | | | |
| Row 2: Eng. SGSH | | | | | | | | |
| Row 3: Eng. | | | | | | | | |
| SGSH w/WPRE | | | | | | | | |
| Row 4: Control | 1° + 2° | 1° + 2° | 2° | | | | | |
| (no virus) | | | only | | | | | |
| Virus MOI: ½ | 100K | 50K | 25K | 12.5K | 6250 | 3125 | 1563 | 781 |
| serial dilution | | | | | | | | |

FIG. 16 shows SGSH transgene expression levels, plotted as AFU (800/700 nm), in neurospheres treated with AAV.hSGSH comprising WT SGSH construct, engineered SGSH construct or engineered SGSH construct further comprising WPRE element in AAV vector genome at various MOI. The data was normalized to account for varying cell mass. The engineered SGSH virus led to less transgene expression than the wild type; this aligns with the above-mentioned in vivo data, whereas transient transfection of the plasmids in HEK cells shows engineered expression to be higher than wild type. These results are encouraging in that the addition of WPRE may significantly boost expression of the engineered construct in vivo.

All documents cited in this specification are incorporated herein by reference. The electronic sequence listing filed herewith named “UPN-19-9075PCT_20221111.xml” with size of 257,646 bytes, created on date of Nov. 11, 2022, and the contents of the electronic sequence listing (e.g., the sequences and text therein) are incorporated herein by reference in entirety. U.S. Provisional Patent Application No. 63/278,775, filed Nov. 12, 2021, U.S. Provisional Patent Application No. 63/288,293, filed Dec. 10, 2021 are incorporated by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) comprising an adeno-associated virus (AAV) capsid and a vector genome, wherein the vector genome comprises an AAV 5′ inverted terminal repeat (ITR), an expression cassette, and an AAV 3′ITR, wherein the expression cassette comprises an engineered nucleic acid sequence encoding for a functional human N-sulfoglycosamine sulfohydrolase (hSGSH), wherein the hSGSH coding sequence comprises a signal peptide sequence and a mature hSGSH coding sequence, wherein the mature hSGSH coding has the nucleic acid sequence of SEQ ID NO: 16 or a nucleic acid sequence which is (a) at least 85% identical to SEQ ID NO: 16; or (b) at least 99% identical to SEQ ID NO: 16, wherein the hSGSH coding sequence is operably linked to regulatory control sequences which direct expression of the hSGSH in a cell.

2. The rAAV according to claim 1, wherein the mature hSGSH coding sequence is 99% identical to SEQ ID NO.

3. The rAAV according to claim 1, wherein the mature hSGSH coding sequence is SEQ ID NO: 16.

4. The rAAV according to claim 1, wherein the mature hSGSH coding sequence has the nucleic acid sequence of sequence of SEQ ID NO: 16 or a nucleic acid sequence which is 85% identical to SEQ ID NO: 16.

5. The rAAV according to claim 1, wherein the signal peptide sequence is a native signal sequence having nucleic acid sequence of SEQ ID NO: 31 or a sequence at least about 95% identical thereto which encodes SEQ ID NO: 32.

6-13. (canceled)

14. The rAAV according to claim 1, wherein the regulatory control sequences comprise a CMV IE enhancer, a chicken-beta actin (CB) promoter, and a chicken beta actin intron.

15. The rAAV according to claim 1, wherein the regulatory control sequences further comprise one or more of a Kozak sequence, an intron, an enhancer, a TATA signal and a polyA sequence.

16. The rAAV according to claim 15, wherein the regulatory control sequences comprise one or more of a chicken beta actin intron, a rabbit globin polyadenylation sequence.

17. The rAAV according to claim 1, wherein the regulatory control sequences further comprise a mutant WPRE element.

18. (canceled)

19-33. (canceled)

34. (canceled)

35. The rAAV according to claim 1, wherein the rAAV vector genome comprises the nucleic acid sequence of SEQ ID NO: 10 (AAV.CB7.CI.hSGSHcoV1.rBG).

36. The rAAV according to claim 1, wherein the rAAV vector genome comprises the nucleic acid sequence of SEQ ID NO: 13 (CB7.CI.hSGSHcoV1-4xmiR183.rBG).

37-38. (canceled)

39. The rAAV according to claim 1, wherein the AAV capsid is a Clade F AAV.

40. The rAAV according to claim 1, wherein the AAV capsid is AAVhu68.

41. The rAAV according to claim 1, wherein the AAV capsid is AAVrh91.

42-44. (canceled)

45. A pharmaceutical composition comprising a rAAV according to claim 1 in a formulation buffer.

46. The pharmaceutical composition according to claim 45, which is formulated for delivery via intracerebroventricular (ICV), intrathecal (IT), intracisternal or intravenous (IV) injection.

47. The pharmaceutical composition according to claim 45, which is administrable at a dose 1×10⁹ GC per gram of brain mass to about 1×10¹³ GC per gram of brain mass.

48-50. (canceled)

51. A nucleic acid molecule comprising an expression cassette comprising an engineered functional human N-sulfoglycosamine sulfohydrolase (hSGSH) gene and regulatory control sequences, said expression cassette being flanked by a 5′ inverted terminal repeat (ITR) and a 3′ ITR, wherein the engineered hSGSG gene encodes a functional hSGSH, wherein the hSGSH coding sequence comprises a signal peptide sequence and a mature hSGSH, and wherein the mature hSGSH-coding sequence is SEQ ID NO: 16.

52-53. (canceled)

54. A packaging host cell comprising a nucleic acid molecule according to claim 51.

55. The packaging host cell according to claim 54 which further comprises AAV rep coding sequences operably linked to sequences which express rep protein in the packaging host cell, an AAV capsid coding sequences operably linked to sequences which express AAV capsid proteins in the packaging host cell, and helper virus functions necessary to permit packaging of the expression cassette and ITRs into the AAV capsid.

56. The packaging host cell according to claim 54, wherein the AAV capsid is selected from a AAVhu68 and AAVrh91.

57. An rAAV production system useful for producing the rAAV according to any of claim 1, wherein the production system comprises a cell culture comprising:
(a) a nucleic acid sequence encoding a AAV capsid protein;
(b) a vector genome; and
(c) sufficient AAV rep functions and helper functions to permit packaging of the vector genome into the AAV capsid.

58. The rAAV production system according to claim 57, wherein the AAV capsid is selected from a AAVhu68 and AAVrh91.

59. The rAAV production system according to claim 57, wherein the vector genome is selected from SEQ ID NO: 10, and 13.

60. A method of treating a human subject diagnosed with MPS IIIA and/or improving gait or mobility, reducing tremors, reducing spasms, improving posture, or reducing the progression of vision loss in a subject in need thereof, comprising administering to the subject a suspension of a rAAV according to claim 1 in a formulation buffer at a dose of 1×10⁹ GC per gram of brain mass to about 1×10¹³ GC per gram of brain mass.

61. The rAAV according to claim 1, wherein the mature hSGSH coding sequence is SEQ ID NO: 35.

62. The rAAV according to claim 1, wherein the rAAV vector genome comprises the nucleic acid sequence of SEQ ID NO: 11 (AAV.CB7.CI.hSGSHcoV1.rBG).


SEQUENCE LISTING (60 sequences)

SEQ ID NO: 1 - DNA, 4738 bp, synthetic construct
  misc_feature 1..4738: note = vector genome CB7.CI.BiP.hSGSHcov1(A482Y-E488V).vIGF2.WPRE.4xmiR183.rBG
  repeat_region 1..130: note = 5'ITR
  regulatory 198..579: note = promoter - CMV IE; regulatory_class = enhancer
  regulatory 582..863: note = promoter - CB promoter; regulatory_class = promoter
  misc_feature 956..1928: note = Intron - chicken beta-actin intron
  misc_feature 1934..3694: note = BiP-hSGSHcoV1-A482Y_E488V_vIGF2
  misc_feature 3383..3385: note = A482Y
  misc_feature 3401..3403: note = E488V
  misc_feature 3446..3688: note = linker vIGF2
  misc_feature 3707..4248: note = WPRE element
  misc_feature 4259..4280: note = miRNA183
  misc_feature 4285..4306: note = miRNA183
  misc_feature 4313..4334: note = miRNA183
  misc_feature 4339..4360: note = miRNA183
  regulatory 4394..4520: note = polyA_signal - rabbit globin polyA; regulatory_class = polyA_signal_sequence
  repeat_region 4609..4738: note = 3' ITR
  source 1..4738: mol_type = other DNA
  regulatory 198..863: note = CB7 promoter; regulatory_class = promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataagagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 4323 bp, synthetic construct
  misc_feature 1..4323: note = expression cassette CB7.CI.BiP.hSGSHcov1(A482Y-E488V).vIGF2.WPRE.4xmiR183.rBG
  regulatory 1..382: note = promoter - CMV; regulatory_class = enhancer
  regulatory 385..666: note = promoter - CB promoter; regulatory_class = promoter
  misc_feature 759..1731: note = Intron - chicken beta actin intron
  misc_feature 3510..4051: note = WPRE
  misc_feature 4062..4083: note = miRNA183
  misc_feature 4088..4109: note = miRNA183
  misc_feature 4116..4137: note = miRNA183
  misc_feature 4142..4163: note = miRNA183
  regulatory 4197..4323: note = polyA_signal - Rabbit globin polyA; regulatory_class = polyA_signal_sequence
  source 1..4323: mol_type = other DNA
  CDS 1737..3494: note = BiP-hSGSHcoV1-A482Y-E488V-vIGF2; protein_id = 3; translation = ASATMKLSLVAAMLLLLSAARARPRNALLLLADDGGFESGAYNN SAIATPHLDALARRSLLFRNAFTSVSSCSPSRASLLTGLPQHQNGMYGLHQDVHHFNS FDKVRSLPLLLSQAGVRTGIIGKKHVGPETVYPFDFAYTEENGSVLQVGRNITRIKLL VRKFLQTQDDRPFFLYVAFHDPHRCGHSQPQYGTFCEKFGNGESGMGRIPDWTPQAYD PLDVLVPYFVPNTPAARADLAAQYTTVGRMDQGVGLVLQELRDAGVLNDTLVIFTSDN GIPFPSGRTNLYWPGTAEPLLVSSPEHPKRWGQVSEAYVSLLDLTPTILDWFSIPYPS YAIFGSKTIHLTGRSLLPALEAEPLWATVFGSQSHHEVTMSYPMRSVQHRHFRLVHNL NFKMPFPIDQDFYVSPTFQDLLNRTTAGQPTGWYKDLRHYYYRARWELYDRSRDPHET QNLATDPRFAQLLEMLRDQLAKWQWETHDPWVCYPDGVLVEKLSPQCQPLHNELRPRA VPTQGGGGSGGGGSSRTLCGGELVDTLQFVCGDRGFLFSRPASRVSRRSRGIVEECCF RSCDLALLETYCATPARSE
  regulatory 1..666: note = CB7 promoter; regulatory_class = promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataagagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 3 - protein, 585 aa, synthetic construct
  REGION 1..585: note = Synthetic Construct
ASATMKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 4 - DNA, 4099 bp, synthetic construct
  misc_feature 1..4099: note = vector genome CB7.CI.BiP.hSGSHcov1-A482Y_E488V.vIGF2.rBG
  repeat_region 1..130: note = 5' ITR
  regulatory 198..579: note = promoter - CMV IE; regulatory_class = enhancer
  regulatory 582..863: note = promoter - CB promoter; regulatory_class = promoter
  misc_feature 956..1928: note = Intron - chicken beta-actin intron
  misc_feature 1934..3700: note = BiP-hSGSHcoV1-A482Y-E488V-vIGF2
  misc_feature 3383..3385: note = A482Y
  misc_feature 3401..3403: note = E488V
  misc_feature 3446..3688: note = linker-vIGF2
  regulatory 3755..3881: note = polyA_signal - Rabbit globin polyA; regulatory_class = polyA_signal_sequence
  repeat_region 3970..4099: note = 3' ITR
  source 1..4099: mol_type = other DNA
  regulatory 198..863: note = CB7 promoter; regulatory_class = promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 5 - DNA, 3684 bp, synthetic construct
  misc_feature 1..3684: note = expression cassette CB7.CI.BiP.hSGSHcov1-A482Y_E488V.vIGF2.rBG
  regulatory 1..382: note = promoter - CMV IE; regulatory_class = enhancer
  regulatory 385..666: note = promoter - CB promoter; regulatory_class = promoter
  misc_feature 759..1731: note = Intron - chicken beta actin promoter
  misc_feature 3186..3188: note = A482Y
  misc_feature 3204..3206: note = E488V
  misc_feature 3249..3491: note = linker vIGF2
  regulatory 3558..3684: note = polyA_signal - rabbit globin polyA; regulatory_class = polyA_signal_sequence
  source 1..3684: mol_type = other DNA
  CDS 1737..3494: note = BiP-hSGSHcoV1-A482Y_E488V_vIGF2; protein_id = 6; translation = ASATMKLSLVAAMLLLLSAARARPRNALLLLADDGGFESGAYNN SAIATPHLDALARRSLLFRNAFTSVSSCSPSRASLLTGLPQHQNGMYGLHQDVHHFNS FDKVRSLPLLLSQAGVRTGIIGKKHVGPETVYPFDFAYTEENGSVLQVGRNITRIKLL VRKFLQTQDDRPFFLYVAFHDPHRCGHSQPQYGTFCEKFGNGESGMGRIPDWTPQAYD PLDVLVPYFVPNTPAARADLAAQYTTVGRMDQGVGLVLQELRDAGVLNDTLVIFTSDN GIPFPSGRTNLYWPGTAEPLLVSSPEHPKRWGQVSEAYVSLLDLTPTILDWFSIPYPS YAIFGSKTIHLTGRSLLPALEAEPLWATVFGSQSHHEVTMSYPMRSVQHRHFRLVHNL NFKMPFPIDQDFYVSPTFQDLLNRTTAGQPTGWYKDLRHYYYRARWELYDRSRDPHET QNLATDPRFAQLLEMLRDQLAKWQWETHDPWVCYPDGVLVEKLSPQCQPLHNELRPRA VPTQGGGGSGGGGSSRTLCGGELVDTLQFVCGDRGFLFSRPASRVSRRSRGIVEECCF RSCDLALLETYCATPARSE
  regulatory 1..666: note = CB7 promoter; regulatory_class = promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 6 - protein, 585 aa, synthetic construct; identical to SEQ ID NO: 3
  REGION 1..585: note = Synthetic Construct

SEQ ID NO: 7 - DNA, 4652 bp, synthetic construct
  misc_feature 1..4652: note = vector genome CB7.CI.BiP.hSGSHcov1-A482Y_E488V.vIGF2.WPRE.rBG
  repeat_region 1..130: note = 5'ITR
  regulatory 198..579: note = promoter - CMV IE; regulatory_class = enhancer
  regulatory 582..863: note = promoter - CB promoter; regulatory_class = promoter
  misc_feature 956..1928: note = Intron - chicken beta actin promoter
  misc_feature 1934..3695: note = BiP-hSGSHcoV1-A482Y-E488V-vIGF2
  misc_feature 3383..3385: note = A482Y
  misc_feature 3401..3403: note = E488V
  misc_feature 3446..3688: note = linker vIGF2
  misc_feature 3707..4248: note = WPRE
  regulatory 4308..4434: note = polyA_signal - Rabbit globin polyA; regulatory_class = polyA_signal_sequence
  repeat_region 4523..4652: note = 3' ITR
  source 1..4652: mol_type = other DNA
  regulatory 198..863: note = CB7 promoter; regulatory_class = promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataagagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 8 - DNA, 4237 bp, synthetic construct
  misc_feature 1..4237: note = expression cassette CB7.CI.BiP.hSGSHcov1-A482Y_E488V.vIGF2.WPRE.rBG
  regulatory 1..382: note = promoter - CMV IE; regulatory_class = enhancer
  regulatory 385..666: note = promoter - CB promoter; regulatory_class = promoter
  misc_feature 759..1731: note = Intron - chicken beta-actin intron
  misc_feature 3186..3188: note = A482Y
  misc_feature 3204..3206: note = E488V
  misc_feature 3249..3491: note = linker vIGF2
  misc_feature 3510..4051: note = WPRE
  regulatory 4111..4237: note = polyA_signal - Rabbit globin polyA; regulatory_class = polyA_signal_sequence
  source 1..4237: mol_type = other DNA
  CDS 1737..3494: note = BiP-hSGSHcoV1-A482Y-E488V-vIGF2; protein_id = 9; translation = ASATMKLSLVAAMLLLLSAARARPRNALLLLADDGGFESGAYNN SAIATPHLDALARRSLLFRNAFTSVSSCSPSRASLLTGLPQHQNGMYGLHQDVHHFNS FDKVRSLPLLLSQAGVRTGIIGKKHVGPETVYPFDFAYTEENGSVLQVGRNITRIKLL VRKFLQTQDDRPFFLYVAFHDPHRCGHSQPQYGTFCEKFGNGESGMGRIPDWTPQAYD PLDVLVPYFVPNTPAARADLAAQYTTVGRMDQGVGLVLQELRDAGVLNDTLVIFTSDN GIPFPSGRTNLYWPGTAEPLLVSSPEHPKRWGQVSEAYVSLLDLTPTILDWFSIPYPS YAIFGSKTIHLTGRSLLPALEAEPLWATVFGSQSHHEVTMSYPMRSVQHRHFRLVHNL NFKMPFPIDQDFYVSPTFQDLLNRTTAGQPTGWYKDLRHYYYRARWELYDRSRDPHET QNLATDPRFAQLLEMLRDQLAKWQWETHDPWVCYPDGVLVEKLSPQCQPLHNELRPRA VPTQGGGGSGGGGSSRTLCGGELVDTLQFVCGDRGFLFSRPASRVSRRSRGIVEECCF RSCDLALLETYCATPARSE
  regulatory 1..666: note = CB7 promoter; regulatory_class = promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataagagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 9 - protein, 585 aa, synthetic construct; identical to SEQ ID NO: 3
  REGION 1..585: note = Synthetic Construct

SEQ ID NO: 10 - DNA, 3862 bp, synthetic construct
  misc_feature 1..3862: note = vector genome CB7.CI.hSGSHcoV1.rBG
  repeat_region 1..130: note = 5' ITR
  regulatory 198..579: note = promoter - CMV IE; regulatory_class = enhancer
  regulatory 582..863: note = promoter - CB promoter; regulatory_class = promoter
  misc_feature 956..1928: note = Intron - chicken beta actin intron
  misc_feature 1946..3451: note = hSGSHcoV1
  regulatory 3518..3644: note = polyA_signal - Rabbit globin polyA; regulatory_class = polyA_signal_sequence
  repeat_region 3733..3862: note = 3' ITR
  source 1..3862: mol_type = other DNA
  regulatory 198..863: note = CB7 promoter; regulatory_class = promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgtcttgtcctgtgcctgcctgttgtgccctgctgctggttctgggactgtgcagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaatgaactgtgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 3447 bp, synthetic construct
  misc_feature 1..3447: note = expression cassette CB7.CI.hSGSHcoV1.rBG
  regulatory 1..382: note = promoter - CMV IE; regulatory_class = enhancer
  regulatory 385..666: note = promoter - CB promoter; regulatory_class = promoter
  misc_feature 759..1731: note = Intron - chicken beta-actin intron
  regulatory 3321..3447: note = polyA_signal - rabbit globin polyA; regulatory_class = polyA_signal_sequence
  source 1..3447: mol_type = other DNA
  CDS 1749..3254: note = hSGSHcoV1; protein_id = 12; translation = MSCPVPACCALLLVLGLCRARPRNALLLLADDGGFESGAYNNSA IATPHLDALARRSLLFRNAFTSVSSCSPSRASLLTGLPQHQNGMYGLHQDVHHFNSFD KVRSLPLLLSQAGVRTGIIGKKHVGPETVYPFDFAYTEENGSVLQVGRNITRIKLLVR KFLQTQDDRPFFLYVAFHDPHRCGHSQPQYGTFCEKFGNGESGMGRIPDWTPQAYDPL DVLVPYFVPNTPAARADLAAQYTTVGRMDQGVGLVLQELRDAGVLNDTLVIFTSDNGI PFPSGRTNLYWPGTAEPLLVSSPEHPKRWGQVSEAYVSLLDLTPTILDWFSIPYPSYA IFGSKTIHLTGRSLLPALEAEPLWATVFGSQSHHEVTMSYPMRSVQHRHFRLVHNLNF KMPFPIDQDFYVSPTFQDLLNRTTAGQPTGWYKDLRHYYYRARWELYDRSRDPHETQN LATDPRFAQLLEMLRDQLAKWQWETHDPWVCAPDGVLEEKLSPQCQPLHNEL
  regulatory 1..666: note = CB7 promoter; regulatory_class = promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgtcttgtcctgtgcctgcctgttgtgccctgctgctggttctgggactgtgcagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaatgaactgtgataaggtacctctagagtcgacccgggcggcctcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 12 - protein, 502 aa, synthetic construct
  REGION 1..502: note = Synthetic Construct
MSCPVPACCALLLVLGLCRARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 13 - DNA, 3944 bp, synthetic construct
  misc_feature 1..3944: note = vector genome CB7.CI.hSGSHcoV1-4xmiR183.rBG
  repeat_region 1..130: note = 5'ITR
  regulatory 198..579: note = promoter - CMV IE; regulatory_class = enhancer
  regulatory 582..863: note = polyA_signal - CB promoter; regulatory_class = polyA_signal_sequence
  misc_feature 956..1928: note = Intron - chicken beta-actin inron
  misc_feature 1946..3451: note = hSGSHcoV1
  misc_feature 3465..3486: note = miRNA183
  misc_feature 3491..3512: note = miRNA183
  misc_feature 3519..3540: note = miRNA183
  misc_feature 3545..3566: note = miRNA183
  regulatory 3600..3726: note = polyA_signal - rabbit globin polyA; regulatory_class = polyA_signal_sequence
  repeat_region 3815..3944: note = 3' ITR
  source 1..3944: mol_type = other DNA
  regulatory 198..863: note = CB7 promoter; regulatory_class = promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgtcttgtcctgtgcctgcctgttgtgccctgctgctggttctgggactgtgcagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaatgaactgtgataagggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 14 - DNA, 3529 bp, synthetic construct
  misc_feature 1..3529: note = expression cassette CB7.CI.hSGSHcoV1-4xmiR183.rBG
  regulatory 1..382: note = promoter - CMV IE; regulatory_class = enhancer
  regulatory 385..666: note = promoter - CB promoter; regulatory_class = promoter
  misc_feature 759..1731: note = Intron - chicken beta actin intron
  misc_feature 3268..3289: note = miRNA183
  misc_feature 3294..3315: note = miRNA183
  misc_feature 3322..3343: note = miRNA183
  misc_feature 3348..3369: note = miRNA183
  regulatory 3403..3529: note = polyA_signal - Rabbit globin polyA; regulatory_class = polyA_signal_sequence
  source 1..3529: mol_type = other DNA
  CDS 1749..3257: note = hSGSHcoV1; protein_id = 15; translation = MSCPVPACCALLLVLGLCRARPRNALLLLADDGGFESGAYNNSA IATPHLDALARRSLLFRNAFTSVSSCSPSRASLLTGLPQHQNGMYGLHQDVHHFNSFD KVRSLPLLLSQAGVRTGIIGKKHVGPETVYPFDFAYTEENGSVLQVGRNITRIKLLVR KFLQTQDDRPFFLYVAFHDPHRCGHSQPQYGTFCEKFGNGESGMGRIPDWTPQAYDPL DVLVPYFVPNTPAARADLAAQYTTVGRMDQGVGLVLQELRDAGVLNDTLVIFTSDNGI PFPSGRTNLYWPGTAEPLLVSSPEHPKRWGQVSEAYVSLLDLTPTILDWFSIPYPSYA IFGSKTIHLTGRSLLPALEAEPLWATVFGSQSHHEVTMSYPMRSVQHRHFRLVHNLNF KMPFPIDQDFYVSPTFQDLLNRTTAGQPTGWYKDLRHYYYRARWELYDRSRDPHETQN LATDPRFAQLLEMLRDQLAKWQWETHDPWVCAPDGVLEEKLSPQCQPLHNEL
  regulatory 1..666: note = CB7 promoter; regulatory_class = promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgtcttgtcctgtgcctgcctgttgtgccctgctgctggttctgggactgtgcagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaatgaactgtgataagggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 15 - protein, 502 aa, synthetic construct; identical to SEQ ID NO: 12
  REGION 1..502: note = Synthetic Construct

SEQ ID NO: 16 - DNA, 1452 bp, synthetic construct
  misc_feature 1..1452: note = engineered nucleic acid sequence encoding mature hSGSH
  source 1..1452: mol_type = other DNA
agagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaatgaactg

SEQ ID NO: 17 - protein, 484 aa, synthetic construct
  REGION 1..484: note = mature hSGSH
RARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 18 - DNA, 1506 bp, synthetic construct
  misc_feature 1..1506: note = engineered nucleic acid sequence encoding BiP.hSGSH
  source 1..1506: mol_type = other DNA
atgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggaccccacaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaatgaactgtgataa

SEQ ID NO: 19 - protein, 500 aa, synthetic construct
  REGION 1..500: note = BiP.hSGSH
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL

SEQ ID NO: 20 - DNA, 1749 bp, synthetic construct
  misc_feature 1..1749: note = engineered nucleic acid sequence encoding BiP.hSGSH.vIGF2
  source 1..1749: mol_type = other DNA
atgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggaccccacaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgacggcgtgctggaagagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataa

SEQ ID NO: 21 - protein, 581 aa, synthetic construct
  REGION 1..581: note = BiP.hSGSH.vIGF2
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 22 - DNA, 1452 bp, synthetic construct
  misc_feature 1..1452: note = engineered nucleic acid sequence encoding mature hSGSH.A482Y-E488V
  source 1..1452: mol_type = other DNA
agagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactg

SEQ ID NO: 23 - protein, 484 aa, synthetic construct
  REGION 1..484: note = mature hSGSH.A482Y-E488V
RARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNEL

SEQ ID NO: 24 - DNA, 1500 bp, synthetic construct
  misc_feature 1..1500: note = engineered nucleic acid sequence encoding BiP.hSGSH.A482Y-E488V
  source 1..1500: mol_type = other DNA
atgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactg

SEQ ID NO: 25 - protein, 500 aa, synthetic construct
  REGION 1..500: note = BiP.hSGSH.A482Y-E488V
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNEL

SEQ ID NO: 26 - DNA, 1749 bp, synthetic construct
  misc_feature 1..1749: note = engineered nucleic acid sequence encoding BiP.hSGSH.A482Y-E488V.vIGF2
  source 1..1749: mol_type = other DNA
atgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataa

SEQ ID NO: 27 - protein, 581 aa, synthetic construct
  REGION 1..581: note = BiP.hSGSH.A482Y-E488V.vIGF2
MKLSLVAAMLLLLSAARARPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCYPDGVLVEKLSPQCQPLHNELRPRAVPTQGGGGSGGGGSSRTLCGGELVDTLQFVCGDRGFLFSRPASRVSRRSRGIVEECCFRSCDLALLETYCATPARSE

SEQ ID NO: 28 - DNA, 22 bp, synthetic construct
  misc_feature 1..22: note = miRNA183 targeting sequence
  source 1..22: mol_type = other DNA
agtgaattctaccagtgccata

SEQ ID NO: 29 - DNA, 48 bp, synthetic construct
  misc_feature 1..48: note = engineered nucleic acid sequence encoding BiP peptide
  source 1..48: mol_type = other DNA
atgaagctgtctctggtggctgctatgctgctgctcctgtctgccgcc

SEQ ID NO: 30 - protein, 16 aa, synthetic construct
  REGION 1..16: note = BiP peptide
MKLSLVAAMLLLLSAA

SEQ ID NO: 31 - DNA, 54 bp, synthetic construct
  misc_feature 1..54: note = engineered nucleic acid sequence encoding hSGSH native signal peptide
  source 1..54: mol_type = other DNA
atgtcttgtcctgtgcctgcctgttgtgccctgctgctggttctgggactgtgc

SEQ ID NO: 32 - protein, 18 aa, synthetic construct
  REGION 1..18: note = hSGSH native signal peptide
MSCPVPACCALLLVLGLC

SEQ ID NO: 33 - DNA, 246 bp, synthetic construct
  misc_feature 1..246: note = engineered nucleic acid sequence encoding vIGF2 peptide
  source 1..246: mol_type = other DNA
agacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatga

SEQ ID NO: 34 - protein, 81 aa, synthetic construct
  REGION 1..81: note = vIGF2 peptide
RPRAVPTQGGGGSGGGGSSRTLCGGELVDTLQFVCGDRGFLFSRPASRVSRRSRGIVEECCFRSCDLALLETYCATPARSE

SEQ ID NO: 35 - DNA, 1509 bp, synthetic construct
  misc_feature 1..1509: note = engineered nucleic acid sequence encoding hSGSH comprising native signal peptide
  source 1..1509: mol_type = other DNA
atgtcttgtcctgtgcctgcctgttgtgccctgctgctggttctgggactgtgcagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgcgctcctgatggcgtgctggaagagaagctgtcccctcagtgtcagcccctgcacaatgaactgtga

SEQ ID NO: 36 - protein, 502 aa, synthetic construct; identical to SEQ ID NO: 12
  REGION 1..502: note = hSGSH comprising native signal peptide

SEQ ID NO: 37 - DNA, 4901 bp, synthetic construct
  misc_feature 1..4901: note = vector genome CB7.CI.Bip.hSGSHcov1-A482Y_E488V.vIGF 2.WPRE.4xmiR182.4xmiR183.rBG .
  repeat_region 1..130: note = 5' ITR
  regulatory 198..579: note = promoter - CMV IE; regulatory_class = enhancer
  regulatory 582..863: note = promoter - CB promoter; regulatory_class = promoter
  misc_feature 956..1928: note = Intron - chicken beta-actin intron
  misc_feature 1934..3694: note = BiP-hSGSHcoV1-A482Y-E488V-vIGF2
  misc_feature 3383..3385: note = A482Y
  misc_feature 3401..3403: note = E488V
  misc_feature 3446..3688: note = linker-vIGF2
  misc_feature 3701..4242: note = WPRE
  misc_feature 4302..4325: note = miR182
  misc_feature 4330..4353: note = miR182
  misc_feature 4360..4383: note = miR182
  misc_feature 4390..4413: note = miR182
  misc_feature 4422..4443: note = miR183
  misc_feature 4448..4469: note = miR183
  misc_feature 4476..4497: note = miR183
  misc_feature 4502..4523: note = miR183
  regulatory 4557..4683: note = polyA_signal - Rabbit beta globin polyA; regulatory_class = polyA_signal_sequence
  repeat_region 4772..4901: note = 3" ITR
  source 1..4901: mol_type = other DNA
  regulatory 198..863: note = CB7 promoter; regulatory_class = promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctctagagtcgacccgggcggcctcgaggacggggtgaactacgcctgaggatccagtgtgagttctaccattgccaaaggatagtgtgagttctaccattgccaaacacgtgagtgtgagttctaccattgccaaagcatgcagtgtgagttctaccattgccaaagcggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 38 - DNA, 4486 bp, synthetic construct
  misc_feature 1..4486: note = expression cassette CB7.CI.Bip.hSGSHcov1-A482Y_E488 V.vIGF2.WPRE.4xmiR182.4xmiR183.rBG
  regulatory 1..382: note = promoter - CMV IE; regulatory_class = enhancer
  regulatory 385..666: note = promoter - CB pormoter; regulatory_class = promoter
  misc_feature 759..1731: note = Intron - chicken beta actin intron
  misc_feature 3186..3188: note = A482Y
  misc_feature 3204..3206: note = E488V
  misc_feature 3249..3491: note = linker vIGF2
  misc_feature 3504..4045: note = WPRE element
  misc_feature 4105..4128: note = miR182
  misc_feature 4133..4156: note = miR182
  misc_feature 4163..4186: note = miR182
  misc_feature 4193..4216: note = miR182
  misc_feature 4225..4246: note = miR183
  misc_feature 4251..4272: note = miR183
  misc_feature 4279..4300: note = miR183
  misc_feature 4305..4326: note = miR183
  regulatory 4360..4486: note = polyA_signal - rabbit globin polyA; regulatory_class = polyA_signal_sequence
  source 1..4486: mol_type = other DNA
  CDS 1737..3494: note = BiP-hSGSHcoV1-A482Y-E488V-vIGF2; protein_id = 39; translation = ASATMKLSLVAAMLLLLSAARARPRNALLLLADDGGFESGAYNN SAIATPHLDALARRSLLFRNAFTSVSSCSPSRASLLTGLPQHQNGMYGLHQDVHHFNS FDKVRSLPLLLSQAGVRTGIIGKKHVGPETVYPFDFAYTEENGSVLQVGRNITRIKLL VRKFLQTQDDRPFFLYVAFHDPHRCGHSQPQYGTFCEKFGNGESGMGRIPDWTPQAYD PLDVLVPYFVPNTPAARADLAAQYTTVGRMDQGVGLVLQELRDAGVLNDTLVIFTSDN GIPFPSGRTNLYWPGTAEPLLVSSPEHPKRWGQVSEAYVSLLDLTPTILDWFSIPYPS YAIFGSKTIHLTGRSLLPALEAEPLWATVFGSQSHHEVTMSYPMRSVQHRHFRLVHNL NFKMPFPIDQDFYVSPTFQDLLNRTTAGQPTGWYKDLRHYYYRARWELYDRSRDPHET QNLATDPRFAQLLEMLRDQLAKWQWETHDPWVCYPDGVLVEKLSPQCQPLHNELRPRA VPTQGGGGSGGGGSSRTLCGGELVDTLQFVCGDRGFLFSRPASRVSRRSRGIVEECCF RSCDLALLETYCATPARSE
  regulatory 1..666: note = CB7 promoter; regulatory_class = promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctctagagtcgacccgggcggcctcgaggacggggtgaactacgcctgaggatccagtgtgagttctaccattgccaaaggatagtgtgagttctaccattgccaaacacgtgagtgtgagttctaccattgccaaagcatgcagtgtgagttctaccattgccaaagcggccgcagtgaattctaccagtgccatacgatagtgaattctaccagtgccataaccggtagtgaattctaccagtgccatatcacagtgaattctaccagtgccatactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 39 - protein, 585 aa, synthetic construct; identical to SEQ ID NO: 3
  REGION 1..585: note = Synthetic Construct

SEQ ID NO: 40 - DNA, 4769 bp, synthetic construct
  misc_feature 1..4769: note = vector genome CB7.CI.BiP.hSGSHcov1-A482Y_E488V.vIGF2.WPRE.4xmiR182.rBG
  repeat_region 1..130: note = 5' ITR
  regulatory 198..579: note = promoter - CMV IE; regulatory_class = enhancer
  regulatory 582..863: note = promoter - CB promoter; regulatory_class = promoter
  misc_feature 956..1928: note = Intron - chicken beta actin intron
  misc_feature 1934..3694: note = BiP-hSGSHcoV1-A482Y-E488V-vIGF2
  misc_feature 3383..3385: note = A482Y
  misc_feature 3401..3403: note = E488V
  misc_feature 3701..4242: note = WPRE element
  misc_feature 4302..4325: note = miR182
  misc_feature 4330..4353: note = miR182
  misc_feature 4360..4383: note = miR182
  misc_feature 4390..4413: note = miR182
  regulatory 4425..4551: note = polyA_signal - rabbit beta globin polyA (RBG); regulatory_class = polyA_signal_sequence
  repeat_region 4640..4769: note = 3' ITR
  source 1..4769: mol_type = other DNA
  regulatory 198..863: note = CB7 promoter; regulatory_class = promoter
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctctagagtcgacccgggcggcctcgaggacggggtgaactacgcctgaggatccagtgtgagttctaccattgccaaaggatagtgtgagttctaccattgccaaacacgtgagtgtgagttctaccattgccaaagcatgcagtgtgagttctaccattgccaaagcggccgatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 41 - DNA, 4354 bp, synthetic construct
  misc_feature 1..4354: note = expression cassette CB7.CI.BiP.hSGSHcov1-A482Y_E488V.vIGF2.WPRE.4xmiR182.rBG
  regulatory 1..382: note = promoter - CMV IE; regulatory_class = enhancer
  regulatory 385..666: note = promoter - CB promoter; regulatory_class = promoter
  misc_feature 759..1731: note = Intron - chicken beta-actin intron
  misc_feature 3186..3188: note = A482Y
  misc_feature 3204..3206: note = E488V
  misc_feature 3249..3491: note = linker vIGF2
  misc_feature 3504..4045: note = WPRE element
  misc_feature 4105..4128: note = miR182
  misc_feature 4133..4156: note = miR182
  misc_feature 4163..4186: note = miR182
  misc_feature 4193..4216: note = miR182
  regulatory 4228..4354: note = polyA_signal - rabbit globin polyA; regulatory_class = polyA_signal_sequence
  source 1..4354: mol_type = other DNA
  CDS 1737..3494: note = BiP-hSGSHcoV1-A482Y-E488V-vIGF2; protein_id = 42; translation = ASATMKLSLVAAMLLLLSAARARPRNALLLLADDGGFESGAYNN SAIATPHLDALARRSLLFRNAFTSVSSCSPSRASLLTGLPQHQNGMYGLHQDVHHFNS FDKVRSLPLLLSQAGVRTGIIGKKHVGPETVYPFDFAYTEENGSVLQVGRNITRIKLL VRKFLQTQDDRPFFLYVAFHDPHRCGHSQPQYGTFCEKFGNGESGMGRIPDWTPQAYD PLDVLVPYFVPNTPAARADLAAQYTTVGRMDQGVGLVLQELRDAGVLNDTLVIFTSDN GIPFPSGRTNLYWPGTAEPLLVSSPEHPKRWGQVSEAYVSLLDLTPTILDWFSIPYPS YAIFGSKTIHLTGRSLLPALEAEPLWATVFGSQSHHEVTMSYPMRSVQHRHFRLVHNL NFKMPFPIDQDFYVSPTFQDLLNRTTAGQPTGWYKDLRHYYYRARWELYDRSRDPHET QNLATDPRFAQLLEMLRDQLAKWQWETHDPWVCYPDGVLVEKLSPQCQPLHNELRPRA VPTQGGGGSGGGGSSRTLCGGELVDTLQFVCGDRGFLFSRPASRVSRRSRGIVEECCF RSCDLALLETYCATPARSE
  regulatory 1..666: note = CB7 promoter; regulatory_class = promoter
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgctagcgccaccatgaagctgtctctggtggctgctatgctgctgctcctgtctgccgccagagccagacctagaaacgctctgctgctgctcgctgacgacggcggatttgagagcggcgcctacaacaacagcgccattgccacacctcacctggacgccctggccagaagaagcctgctgttcagaaacgccttcaccagcgtgtccagctgcagcccttctagagcctctctgctgactggactgccccagcaccagaacgggatgtacggcctgcaccaggacgtgcaccacttcaacagcttcgacaaagtgcggagcctgcctctgcttctgtctcaagccggcgtcagaacaggcatcatcggcaagaaacacgtgggccccgagacagtgtaccccttcgatttcgcctacaccgaagagaacggcagcgtgctgcaagtgggcagaaacatcacccggatcaagctgctcgtgcggaagttcctgcagacccaggacgacagacccttcttcctgtacgtggccttccacgatcctcacagatgcggccatagccagcctcagtacggcaccttctgcgagaagtttggcaacggcgagagcggcatgggcagaatccctgattggacccctcaggcctacgatcccctggacgtgctggtgccttacttcgtgcctaacacaccagccgccagagccgatctggccgctcagtatacaaccgtgggaagaatggaccaaggcgtcggcctggttctgcaagagcttagagatgccggcgtgctgaacgacaccctggtcatctttaccagcgacaacggcatcccctttccatctggccggaccaatctgtactggcctggaacagctgagcccctgctggtgtctagccctgagcaccctaagagatggggccaagtgtctgaggcctacgtgtccctgctggatctgacccctaccatcctggactggttcagcatcccctatcctagctacgccatcttcggcagcaagaccatccacctgaccggcagatctctgctgccagctctggaagctgaacctctgtgggccacagtgtttggcagccagtctcaccacgaagtgaccatgagctaccccatgagaagcgtgcagcaccggcacttcagactggtgcacaacctgaacttcaagatgccctttccaatcgaccaggacttctacgtgtccccaaccttccaggacctgctgaacagaaccacagccggccaacctaccggctggtacaaggacctgcggcactactactatagagccaggtgggagctgtacgaccggtccagagatccccacgagacacagaacctggccaccgatcctagattcgcccagctgctggaaatgctgagagatcagctggccaagtggcagtgggagacacacgatccttgggtctgctatcctgatggcgtgctagtggagaagctgtcccctcagtgtcagcccctgcacaatgaactgagacctagagctgtgcctacacagggtggcggaggatctggcggaggcggatctagcagaacactttgtggcggagagctggtggacaccctgcagtttgtgtgtggcgacagaggcttcctgttcagcagacctgccagccgggtttccagacggtctagaggaatcgtggaagagtgctgcttcagaagctgcgatctggccctgctggaaacctactgtgccacaccagccagatctgaatgataaggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgagatctctagagtcgacccgggcggcctcgaggacggggtgaactacgcctgaggatccagtgtgagttctaccattgccaaaggatagtgtgagttctaccattgccaaacacgtgagtgtgagttctaccattgccaaagcatgcagtgtgagttctaccattgccaaagcggccgatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 42 - protein, 585 aa, synthetic construct; identical to SEQ ID NO: 3
  REGION 1..585: note = Synthetic Construct

SEQ ID NO: 43 - DNA, 542 bp, synthetic construct
  misc_feature 1..542: note = WPRE element (mut)
  source 1..542: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 44 - DNA, 666 bp, synthetic construct
  misc_feature 1..666: note = CB7 promoter
  source 1..666: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 45 - DNA, 973 bp, synthetic construct
  misc_feature 1..973: note = Chicken beta actin intron
  source 1..973: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 46 - DNA, 127 bp, synthetic construct
  misc_feature 1..127: note = rabbit beta globin polyA (RBG)
  source 1..127: mol_type = other DNA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 47 - DNA, 24 bp, synthetic construct
  misc_feature 1..24: note = miR182 targeting sequence
  source 1..24: mol_type = other DNA
agtgtgagttctaccattgccaaa

SEQ ID NO: 48 - DNA, 2211 bp, adeno-associated virus hu68
  source 1..2211: mol_type = other DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 49 - DNA, 2211 bp, synthetic construct
  source 1..2211: mol_type = other DNA; note = adeno-associated virus hu68 engineered nucleic acid sequence
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 50 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - DNA, 2211 bp, adeno-associated virus rh91
  source 1..2211: mol_type = other DNA
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 52 - DNA, 2211 bp, synthetic construct
  source 1..2211: mol_type = other DNA; note = adeno-associated virus rh91 engineered nucleic acid sequence
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 53 - protein, 736 aa, adeno-associated virus rh91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 54 - DNA, 2211 bp, adeno-associated virus hu95
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagtttcaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaataatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaagccgtgggtcgttcgtccttttactgcctggaatatttcccgtcgcaaatgcttcgtaccggaaacaactttaccttcagctacgagtttgagaacgtacctttccatagcagctatgctcacagccaaagcctggaccgtctcatgaatccactcatcgaccaatacttgtactatctctcaaagactattaacggttctggacagaatcaacaaacgctaaaattcagtgtggccggacccagcaacatggcagtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcttatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagttagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 55 - DNA, 2211 bp, synthetic construct
  source 1..2211: mol_type = other DNA; note = adeno-associated virus hu95 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagcgggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccagctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 56 - protein, 736 aa, adeno-associated virus hu95
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 57 - DNA, 2211 bp, adeno-associated virus hu96
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgccgcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgagggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagttatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgcttcgtaccggaaacaactttaccttcagctacgagtttgagaacgtacctttccatagcagctacgctcacagccaaagcctggaccgactaatgaatccactcatcgaccaatacttgtactatctctcaaagactattaacggttctgggcagaatcaacaaacgctaaaattcagtgtggccggacccagcaacatggcagtccagggaagaaactacatacctggacccatctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccggtagcaacggagtcctatggacaagtggccacaaaccaccagagtgcccaagcacaggcgcaaacaggctgggttcaaaaccaaggaatacttccgggtatggtttggcaagacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 58 - DNA, 2211 bp, synthetic construct
  source 1..2211: mol_type = other DNA; note = adeno-associated virus hu96 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagagggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccatctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 59 - protein, 736 aa, adeno-associated virus hu96
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSEGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPIYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 60 - DNA, 1509 bp, Homo sapiens
  source 1..1509: mol_type = other DNA; note = Wild-Type hSGSH coidng sequence
atgagctgccccgtgcccgcctgctgcgcgctgctgctagtcctggggctctgccgggcgcgtccccggaacgcactgctgctcctcgcggatgacggaggctttgagagtggcgcgtacaacaacagcgccatcgccaccccgcacctggacgccttggcccgccgcagcctcctctttcgcaatgccttcacctcggtcagcagctgctctcccagccgcgccagcctcctcactggcctgccccagcatcagaatgggatgtacgggctgcaccaggacgtgcaccacttcaactccttcgacaaggtgcggagcctgccgctgctgctcagccaagctggtgtgcgcacaggcatcatcgggaagaagcacgtggggccggagaccgtgtacccgtttgactttgcgtacacggaggagaatggctccgtcctccaggtggggcggaacatcactagaattaagctgctcgtccggaaattcctgcagactcaggatgaccggcctttcttcctctacgtcgccttccacgacccccaccgctgtgggcactcccagccccagtacggaaccttctgtgagaagtttggcaacggagagagcggcatgggtcgtatcccagactggaccccccaggcctacgacccactggacgtgctggtgccttacttcgtccccaacaccccggcagcccgagccgacctggccgctcagtacaccaccgtcggccgcatggaccaaggagttggactggtgctccaggagctgcgtgacgccggtgtcctgaacgacacactggtgatcttcacgtccgacaacgggatccccttccccagcggcaggaccaacctgtactggccgggcactgctgaacccttactggtgtcatccccggagcacccaaaacgctggggccaagtcagcgaggcctacgtgagcctcctagacctcacgcccaccatcttggattggttctcgatcccgtaccccagctacgccatctttggctcgaagaccatccacctcactggccggtccctcctgccggcgctggaggccgagcccctctgggccaccgtctttggcagccagagccaccacgaggtcaccatgtcctaccccatgcgctccgtgcagcaccggcacttccgcctcgtgcacaacctcaacttcaagatgccctttcccatcgaccaggacttctacgtctcacccaccttccaggacctcctgaaccgcaccacagctggtcagcccacgggctggtacaaggacctccgtcattactactaccgggcgcgctgggagctctacgaccggagccgggacccccacgagacccagaacctggccaccgacccgcgctttgctcagcttctggagatgcttcgggaccagctggccaagtggcagtgggagacccacgacccctgggtgtgcgcccccgacggcgtcctggaggagaagctctctccccagtgccagcccctccacaatgagctgtga